(19) United States
(12) Patent Application Publication
(10) Pub. No.: US 2026/0116893 A1
(43) Pub. Date: Apr. 30, 2026

(54) ORGANIC COMPOUND

(71) Applicant: SEMICONDUCTOR ENERGY LABORATORY CO., LTD., Atsugi-shi (JP)

(72) Inventors: Yuko KUBOTA, Atsugi (JP); Yui YOSHIYASU, Atsugi (JP); Nobuharu OHSAWA, Zama (JP); Shinya FUKUZAKI, Atsugi (JP)

(21) Appl. No.: 19/355,486

(22) Filed: Oct. 10, 2025

(30) Foreign Application Priority Data
Oct. 25, 2024 (JP) 2024-188212

(51) Int. Cl. C07D 519/00; H10K 50/16; H10K 50/19; H10K 85/60

(52) U.S. Cl. CPC C07D 519/00; H10K 85/6572; H10K 50/16; H10K 50/19

(57) ABSTRACT

To provide a novel phenanthroline derivative. An organic compound represented by General Formula (G1) below is provided. In General Formula (G1), A¹ and A² each independently represent a group represented by General Formula (g1) below. In General Formula (g1) below, R¹¹ to R¹⁸ each independently represent hydrogen (including deuterium) or a substituent, and p and q each independently represent 0 to 3. Ar¹ represents a divalent group, and n represents an integer of 0 to 3. When n is 2 or more, a plurality of Ar¹s may be the same or different from each other.

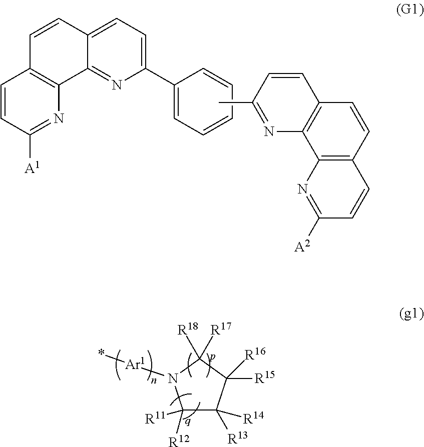

### BACKGROUND OF THE INVENTION

### 1. Field of the Invention

One embodiment of the present invention relates to an organic compound, an organic electronic device, a light-emitting device, an organic EL device, an electronic appliance, and a method for synthesizing an organic compound.

Note that one embodiment of the present invention is not limited to the above technical field. Examples of the technical field of one embodiment of the present invention include a compound, a light-emitting device, an organic EL device, a semiconductor device, a display device, a light-emitting apparatus, a power storage device, a memory device, an electronic appliance, a lighting device, an input device (e.g., a touch sensor), an input/output device (e.g., a touch panel), a method for driving any of them, and a method for manufacturing any of them.

### 2. Description of the Related Art

Recently, display devices have been expected to be applied to a variety of uses. Usage examples of large-sized display devices include a television device for home use (also referred to as TV or television receiver), digital signage, and a public information display (PID). In addition, a smartphone and a tablet terminal each including a touch panel, for example, are being developed as portable information terminals.

At the same time, an increase in the resolution of display devices is also required. Devices for virtual reality (VR), augmented reality (AR), substitutional reality (SR), or mixed reality (MR) given as examples of devices requiring high-resolution display devices have been actively developed.

Light-emitting apparatuses including light-emitting devices (also referred to as light-emitting elements) using organic compounds have been developed as display devices. Light-emitting devices utilizing electroluminescence (hereinafter referred to as EL; such devices are also referred to as organic EL devices or light-emitting devices) have features such as ease of reduction in thickness and weight, high-speed response to input signals, and driving with a constant voltage DC power source, and have been used in display devices.

Patent Document 1 discloses a light-emitting device with a low driving voltage and high reliability that includes an electron-injection layer formed using a mixed film of a transition metal and an organic compound including an unshared electron pair.

### REFERENCE

[Patent Document 1] Japanese Published Patent Application No. 2018-201012

### Non-Patent Document

[Non-Patent Document 1] Fukuzaki S, et al. “High-luminance and Highly Reliable Tandem OLED Display Including New Intermediate Connector Designed for Photolithography Applications”, Journal of the Society for Information Display. 2024. https://doi.org/10.1002/jsid.1294

### SUMMARY OF THE INVENTION

An object of one embodiment of the present invention is to provide a novel phenanthroline derivative. An object of another embodiment of the present invention is to provide a novel phenanthroline derivative having high heat resistance. An object of another embodiment of the present invention is to provide a novel phenanthroline derivative that is suitably used for an intermediate layer of a tandem light-emitting device.

Furthermore, an object of one embodiment of the present invention is to provide a novel light-emitting device, a novel display device, a novel display module, and a novel electronic appliance.

Note that the description of these objects does not preclude the presence of other objects. One embodiment of the present invention does not necessarily achieve all of these objects. Other objects can be derived from the description of the specification, the drawings, and the claims.

In accordance with one embodiment of the present invention, a phenanthroline dimer having an electron-donating group is provided. The organic compound has high heat resistance and stability and can be used for an intermediate layer of a tandem light-emitting device. Since a light-emitting device using the organic compound for the intermediate layer has favorable heat resistance, deterioration due to preservation and driving at high temperatures can be inhibited. One embodiment of the present invention having such a feature is an organic compound represented by General Formula (G1) below.

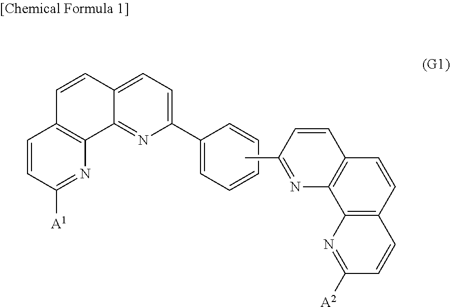

Note that in General Formula (G1) above, A¹ and A² each independently represent a group represented by General Formula (g1) below.

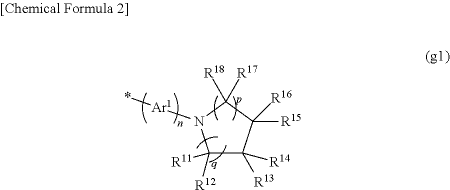

In General Formula (g1) above, R¹¹ to R¹⁸ each independently represent any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p and q each independently represent 0 to 3. Any two of R¹¹ to R¹⁸ may be bonded to each other to form a ring. The aliphatic cyclic amino group represented by General Formula (g1) above may be condensed with an aromatic ring having 6 to 10 carbon atoms. Ar¹ represents any one of a substituted or unsubstituted alkylene group having 1 to 3 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms, and n represents an integer of 0 to 3. When n is 2 or more, a plurality of Ar¹s may be the same or different from each other.

Another embodiment of the present invention is an organic compound represented by General Formula (G2).

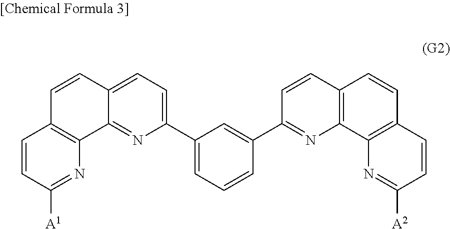

Note that in General Formula (G2) above, A¹ and A² each independently represent a group represented by General Formula (g1) below.

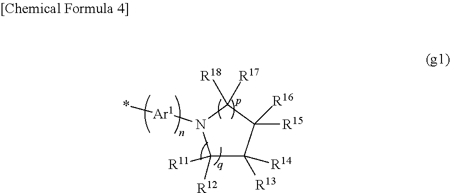

In General Formula (g1) above, R¹¹ to R⁸ each independently represent any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p and q each independently represent 0 to 3. Any two of R¹¹ to R¹⁸ may be bonded to each other to form a ring. The aliphatic cyclic amino group represented by General Formula (g1) above may be condensed with an aromatic ring having 6 to 10 carbon atoms. Ar¹ represents any one of a substituted or unsubstituted alkylene group having 1 to 3 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms, and n represents an integer of 0 to 3. When n is 2 or more, a plurality of Ar¹s may be the same or different from each other.

Another embodiment of the present invention is an organic compound in which n is 0 in the above structure.

Another embodiment of the present invention is an organic compound in which R¹¹, R¹², R¹⁷, and R¹⁸ represent hydrogen in the above structure.

Another embodiment of the present invention is an organic compound in which p and q are 1 in the above structure.

Another embodiment of the present invention is an organic compound represented by Structural Formula (100) below.

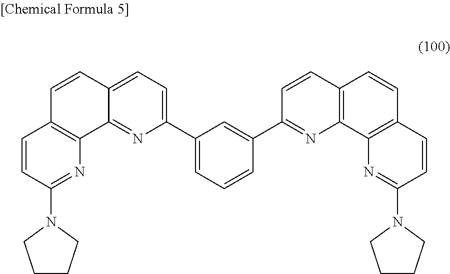 [SMILES: c1cc(-c2ccc3ccc4ccc(N5CCCC5)nc4c3n2)cc(-c2ccc3ccc4ccc(N5CCCC5)nc4c3n2)c1]

Another embodiment of the present invention is an organic semiconductor device including any of the organic compounds described above.

Another embodiment of the present invention is a light-emitting device including any of the organic compounds described above.

Another embodiment of the present invention is a light-receiving device including any of the organic compounds described above.

Another embodiment of the present invention is an organic electronic device using any of the organic compounds described above for a cap layer.

Another embodiment of the present invention is an electronic appliance including the above organic electronic device.

According to one embodiment of the present invention, a novel phenanthroline derivative can be provided. According to another embodiment of the present invention, a novel phenanthroline derivative having high heat resistance can be provided. According to another embodiment of the present invention, a novel phenanthroline derivative that is suitably used for an intermediate layer in a tandem light-emitting device can be provided.

According to one embodiment of the present invention, a novel light-emitting device, a novel display device, a novel display module, and a novel electronic appliance can be provided.

Note that the description of these effects does not preclude the presence of other effects. One embodiment of the present invention does not necessarily have all of these effects. Other effects can be derived from the description of the specification, the drawings, and the claims.

### BRIEF DESCRIPTION OF THE DRAWINGS

In the accompanying drawings:

FIGS. 1A to 1C are schematic views of a light-emitting device of one embodiment of the present invention;

FIGS. 2A and 2B illustrate a display device of one embodiment of the present invention;

FIGS. 3A and 3B illustrate a display device of one embodiment of the present invention;

FIGS. 4A to 4E are cross-sectional views illustrating an example of a method for manufacturing a display device;

FIGS. 5A and 5B are cross-sectional views illustrating an example of a method for manufacturing a display device;

FIGS. 6A to 6D are cross-sectional views illustrating an example of a method for manufacturing a display device;

FIGS. 7A to 7C are cross-sectional views illustrating an example of a method for manufacturing a display device;

FIGS. 8A to 8C are cross-sectional views illustrating an example of a method for manufacturing a display device;

FIGS. 9A to 9C are cross-sectional views illustrating an example of a method for manufacturing a display device;

FIGS. 10A and 10B are perspective views illustrating a structure example of a display module;

FIGS. 11A and 11B are cross-sectional views illustrating structure examples of a display device;

FIG. 12 is a perspective view illustrating a structure example of a display device;

FIG. 13 is a cross-sectional view illustrating a structure example of a display device;

FIG. 14 is a cross-sectional view illustrating a structure example of a display device;

FIG. 15A is a cross-sectional view illustrating a structure example of a display device, and FIGS. 15B and 15C are top-view layouts of pixels;

FIG. 16 is a cross-sectional view illustrating a structure example of a display device;

FIG. 17A is a cross-sectional view illustrating a structure example of a display device, FIG. 17B is a top-view layout of pixels, and FIG. 17C is a top-view layout of pixels and microlenses;

FIGS. 18A to 18D illustrate examples of wearable devices;

FIGS. 19A to 19F illustrate examples of electronic appliances;

FIGS. 20A to 20G illustrate examples of electronic appliances;

FIGS. 21A to 21C show ¹H NMR spectrum of mPrdPhen2P;

FIG. 22 shows luminance-current density characteristics of light-emitting devices 1-1 and 1-2 and comparative light-emitting devices 1-1 and 1-2;

FIG. 23 shows current efficiency-luminance characteristics of the light-emitting devices 1-1 and 1-2 and the comparative light-emitting devices 1-1 and 1-2;

FIG. 24 shows luminance-voltage characteristics of the light-emitting devices 1-1 and 1-2 and the comparative light-emitting devices 1-1 and 1-2;

FIG. 25 shows current density-voltage characteristics of the light-emitting devices 1-1 and 1-2 and the comparative light-emitting devices 1-1 and 1-2;

FIG. 26 shows electroluminescence spectra of the light-emitting devices 1-1 and 1-2 and the comparative light-emitting devices 1-1 and 1-2;

FIG. 27 shows luminance-current density characteristics of a light-emitting device 2 and a comparative light-emitting device 2;

FIG. 28 shows current efficiency-luminance characteristics of the light-emitting device 2 and the comparative light-emitting device 2;

FIG. 29 shows luminance-voltage characteristics of the light-emitting device 2 and the comparative light-emitting device 2;

FIG. 30 shows current density-voltage characteristics of the light-emitting device 2 and the comparative light-emitting device 2; and

FIG. 31 shows electroluminescence spectra of the light-emitting device 2 and the comparative light-emitting device 2.

### DETAILED DESCRIPTION OF THE INVENTION

Embodiments will be described in detail with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

Ordinal numbers such as “first” and “second” in this specification and the like are used in order to avoid confusion among components and do not denote any particular order or ranking such as the order of steps or the stacking order. A term without an ordinal number in this specification and the like may be provided with an ordinal number in a claim in order to avoid confusion among components. A term with an ordinal number in this specification and the like may be provided with a different ordinal number in a claim. A term with an ordinal number in this specification and the like may not be provided with an ordinal number in a claim.

### Embodiment 1

In recent years, display devices including light-emitting devices (which are synonymous with organic EL devices in this specification) have been put into practical use, which facilitates realization, research, and development of useful technologies.

For example, a tandem light-emitting device which includes a plurality of light-emitting units between a pair of electrodes and includes an intermediate layer that generates electric charges between the plurality of light-emitting units can have higher current efficiency than a light-emitting device which includes only one light-emitting unit between electrodes (also referred to as a single-structure light-emitting device).

The intermediate layer in the tandem light-emitting device includes a charge-generation layer (CGL). The CGL refers to a layer where electrons and holes are generated by charge separation upon voltage application.

Preferably, as the CGL, stacking of a layer including a material having an electron-transport property and a material having an electron-donor property to the material having an electron-transport property (n-type layer: a first layer) and a layer including a material having a hole-transport property and a material having an electron-acceptor property to the material having a hole-transport property (p-type layer: a second layer) facilitates injection of electrons or holes to each light-emitting unit, leading to lower the driving voltage.

On the other hand, a photolithography method enables not only formation of fine patterns but also easy processing of a larger area, as compared to mask vapor deposition. Thus, research on the processing of organic compound films using the photolithography method as a substitute for mask vapor deposition in the manufacture of light-emitting devices has been promoted.

In the case where a tandem light-emitting device is intended to be processed by the photolithography method, a material having an electron-donor property to an electron-transport material, typically an alkali metal, an alkaline earth metal, or a compound thereof or the like (hereinafter also referred to as an “alkali metal compound or the like”) is generally used for the n-type layer. The alkali metal compound or the like is highly reactive with water or oxygen and thus rapidly deteriorates not only when directly exposed to the air but also when exposed to the air through a plurality of organic compound layers. As a result, the electron-donor property is impaired. Accordingly, the tandem light-emitting device subjected to the processing by a photolithography method, which requires exposure of the surface of an EL layer to the air in the processing, has an increased driving voltage and has difficulty in exhibiting favorable characteristics.

By contrast, when a layer including a metal or a metal compound and an organic compound having a phenanthroline ring is used as the n-type layer in the intermediate layer, a tandem light-emitting device having favorable characteristics can be obtained even through a photolithography process involving exposure of the EL layer to the air (see e.g., Non-Patent Document 1).

Here, the processing by a photolithography method often involves a heating step to remove moisture. An in-vehicle display and the like are sometimes placed in an environment where they are exposed to high temperatures for a long time. In other words, a light-emitting device preferably has higher heat resistance.

In view of the above, one embodiment of the present invention provides an organic compound including two phenanthroline skeletons and a group including aliphatic cyclic amine, as an organic compound that can be used for an n-type layer of a tandem light-emitting device, enables favorable characteristics of the tandem light-emitting device to be maintained even when the light-emitting device is processed by a photolithography method, and enables the light-emitting device to have higher heat resistance.

In other words, one embodiment of the present invention provides an organic compound represented by General Formula (G1).

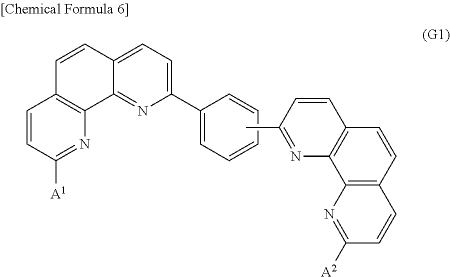

In General Formula (G1) above, A¹ and A² each independently represent a group represented by General Formula (g1) below.

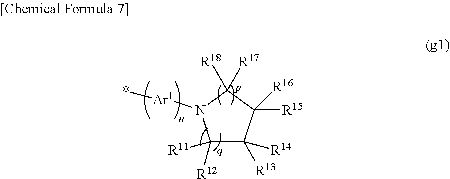

In the aliphatic cyclic amino group represented by General Formula (g1) above, R¹¹ to R¹⁸ each independently represent any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group. Any two of R¹¹ to R¹⁸ may be bonded to each other to form a ring.

In the aliphatic cyclic amino group represented by General Formula (g1) above, p and q each independently represent 0 to 3.

The aliphatic cyclic amino group represented by General Formula (g1) above may be condensed with an aromatic ring having 6 to 10 carbon atoms.

In the aliphatic cyclic amino group represented by General Formula (g1) above, Ar¹ represents any one of a substituted or unsubstituted alkylene group having 1 to 3 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms, and n represents an integer of 0 to 3. When n is 2 or more, a plurality of Ar¹s may be the same or different from each other.

Note that in the organic compound represented by General Formula (G1) above, two phenanthroline skeletons are preferably bonded at the meta-position on a central benzene ring, in which case a film with low crystallinity can be maintained and crystallization at high temperatures can be inhibited. Furthermore, the meta-substituted compound can have a lower sublimation temperature than that a para-substituted compound; thus, a compound having both high sublimation property and high heat resistance can be provided. That is, one embodiment of the present invention is preferably an organic compound represented by General Formula (G2).

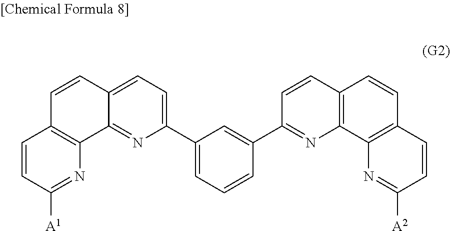

Note that in General Formula (G2) above, A¹ and A² each independently represent an aliphatic cyclic amino group represented by General Formula (g1) above.

In the organic compounds represented by General Formulas (G1), (G2), and (g1) above, examples of the alkyl group having 1 to 10 carbon atoms include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a heptyl group, an octyl group, a 3-methylpentyl group, a 2-methylpentyl group, a 2-ethylbutyl group, a 1,2-dimethylbutyl group, a 2,3-dimethylbutyl group, a 2-ethylhexyl group, a 1-ethylpropyl group, a nonyl group, a 3,7-dimethyl-1-octyl group, a 3,7-dimethyl-2-octyl group, and a decyl group. Note that a tert-butyl group or a cyclohexyl group is particularly preferable because the refractive index can be reduced.

In the organic compounds represented by General Formulas (G1), (G2), and (g1) above, examples of the cycloalkyl group having 3 to 10 carbon atoms include a cyclopropyl group, a cyclobutyl group, a cyclopentyl group, a cyclohexyl group, a cycloheptyl group, a cyclooctyl group, a cyclononyl group, a cyclodecyl group, an adamantyl group, a bicyclo[2.2.1]heptyl group, a tricyclo[5.2.1.0(2,6)]decyl group, a noradamantyl group, a 1-methylcyclohexyl group, an adamantyl group, a bicyclo[2,2,2]octyl group, and a norbornyl group.

In General Formulas (G1), (G2), and (g1) above, examples of the alkoxy group having 1 to 10 carbon atoms include a methoxy group, an ethoxy group, an n-propoxy group, an isopropoxy group, an n-butoxy group, a sec-butoxy group, an isobutoxy group, a tert-butoxy group, an n-pentyloxy group, an isopentyloxy group, a sec-pentyloxy group, a tert-pentyloxy group, a neopentyloxy group, an n-hexyloxy group, an isohexyloxy group, a sec-hexyloxy group, a tert-hexyloxy group, a neohexyloxy group, and a cyclohexyloxy group.

In General Formulas (G1), (G2), and (g1) above, a cyclic secondary amine is preferably used as the secondary amino group having 2 to 10 carbon atoms; examples include a pyrrolidin-1-yl group, an isoindol-2-yl group, a dihydroisoindol-2-yl group, a tetrahydroisoindol-2-yl group, a hexahydroisoindol-2-yl group, a hexahydroisoindolin-2-yl group, a piperidin-1-yl group, an aziridin-1-yl group, an azetidin-1-yl group, an octahydrocyclopenta[c]pyrrol-2-yl group, an octahydro-4,7-methano-1H-isoindol-2-yl group, a 2-azabicyclo[3.1.0]hexan-2-yl group, a 3-azabicyclo[3.1.0]hexan-2-yl group, a 3-azabicyclo[3.2.0]heptan-2-yl group, a 5-azaspiro[3.4]octan-5-yl group, an 8-azabicyclo[3.2.1]octan-8-yl group, a 7-azabicyclo[2.2.1]heptan-7-yl group, a 5-azaspiro[2.4]heptan-5-yl group, a 5-azabicyclo[2.1.1]hexan-5-yl group, a dimethylamino group, a diethylamino group, a diisopropylamino group, a diphenylamino group, and a dicyclohexylamino group. In the case where the cyclic secondary amino group having 2 to 10 carbon atoms has a substituent, examples of the substituent include an alkyl group having 1 to 4 carbon atoms, a cycloalkyl group having 3 to 6 carbon atoms, and an aryl group having 6 to 13 carbon atoms.

Examples of the monovalent aromatic hydrocarbon group having 6 to 30 carbon atoms include a phenyl group, an o-tolyl group, an m-tolyl group, a p-tolyl group, a mesityl group, a biphenyl-2-yl group (o-biphenyl group), a biphenyl-3-yl group (m-biphenyl group), a biphenyl-4-yl group (p-biphenyl group), a 1-naphthyl group, a 2-naphthyl group, a phenylnaphthyl group, a naphthylphenyl group, a terphenyl group, a fluorenyl group, a 9,9-dimethylfluorenyl group, a quaterphenyl group, a spirobifluorenyl group, a phenanthryl group, an anthryl group, a binaphthylphenyl group, a fluoranthenyl group, and a triphenylenyl group. In the case where the aryl group having 6 to 30 carbon atoms has a substituent, examples of the substituent include an alkyl group having 1 to 4 carbon atoms, a cycloalkyl group having 3 to 6 carbon atoms, an aryl group having 6 to 13 carbon atoms, a halogen, and a cyano group.

Specific examples of the heteroaryl group having 1 to 30 carbon atoms include a 1,3,5-triazin-2-yl group, a 1,2,4-triazin-3-yl group, a pyrimidin-4-yl group, a pyrazin-2-yl group, a 2-pyridyl group, a 3-pyridyl group, a 4-pyridyl group, a carbazolyl group, a dibenzofuranyl group, a dibenzothiophenyl group, a benzonaphthofuranyl group, a benzonaphthothiophenyl group, a dinaphthofuranyl group, a dinaphthothiophenyl group, an indolocarbazolyl group, a benzofurocarbazolyl group, a benzothienocarbazolyl group, an indenocarbazolyl group, a dibenzocarbazolyl group, an indolyl group, a pyrrolyl group, a 1,2,3-triazol-yl group, and a 1,2,4-triazol-yl group. In the case where the heteroaryl group having 1 to 30 carbon atoms has a substituent, examples of the substituent include an alkyl group having 1 to 4 carbon atoms, a cycloalkyl group having 3 to 6 carbon atoms, an aromatic hydrocarbon group having 6 to 13 carbon atoms, a halogen, and a cyano group.

Examples of the alkylene group having 1 to 3 carbon atoms include a methylene group, an ethylene group, and a propylene group. As the cycloalkylene group having 3 to 10 carbon atoms, a divalent group obtained by removing one hydrogen atom from the above-mentioned cycloalkyl group having 3 to 10 carbon atoms can be used, for example.

Examples of the divalent heterocyclic group having 1 to 25 carbon atoms include a pyrimidine-diyl group, a pyrazine-diyl group, a pyridazine-diyl group, a triazine-diyl group, a bipyridine-diyl group, a phenanthroline-diyl group, a quinoxaline-diyl group, a dibenzoquinoxaline-diyl group, a quinazoline-diyl group, a benzoquinazoline-diyl group, a dibenzoquinazoline-diyl group, an imidazole-diyl group, a triazole-diyl group, an oxadiazole-diyl group, a benzimidazole-diyl group, a furodiazine-diyl group, a benzofuropyrimidine-diyl group, a thiophene-diyl group, a furan-diyl group, a benzothiophene-diyl group, a benzofuran-diyl group, a dibenzothiophene-diyl group, a dibenzofuran-diyl group, a benzonaphthothiophene-diyl group, a benzonaphthofuran-diyl group, a dinaphthothiophene-diyl group, a dinaphthofuran-diyl group, a piperazine-diyl group, a hexahydropyrimidine-diyl group, a hexahydrotriazine-diyl group, a decahydroquinoxaline-diyl group, a decahydronaphthyridine-diyl group, an imidazolidine-diyl group, an octahydropyrrolopyridine-diyl group, and an octahydropyrrolopyrrole-diyl group. In the case where the divalent heterocyclic group having 1 to 25 carbon atoms has a substituent, examples of the substituent include an alkyl group having 1 to 4 carbon atoms, a cycloalkyl group having 3 to 6 carbon atoms, and an aryl group having 6 to 13 carbon atoms.

Examples of the divalent aromatic hydrocarbon group having 6 to 25 carbon atoms include a phenylene group, a biphenyl-diyl group, a naphthalene-diyl group, a fluorene-diyl group, an acenaphthene-diyl group, an anthracene-diyl group, a phenanthrene-diyl group, a terphenyl-diyl group, a triphenylene-diyl group, a tetracene-diyl group, a benzanthracene-diyl group, a pyrene-diyl group, and a spirobi[9H-fluorene]-diyl group. In the case where the arylene group having 6 to 30 carbon atoms has a substituent, examples of the substituent include an alkyl group having 1 to 4 carbon atoms, a cycloalkyl group having 3 to 6 carbon atoms, and an aryl group having 6 to 13 carbon atoms.

As the aliphatic cyclic amino group represented by General Formula (g1) above, groups represented by Structural Formulas (Am-1) to (Am-49) below are preferable, for example.

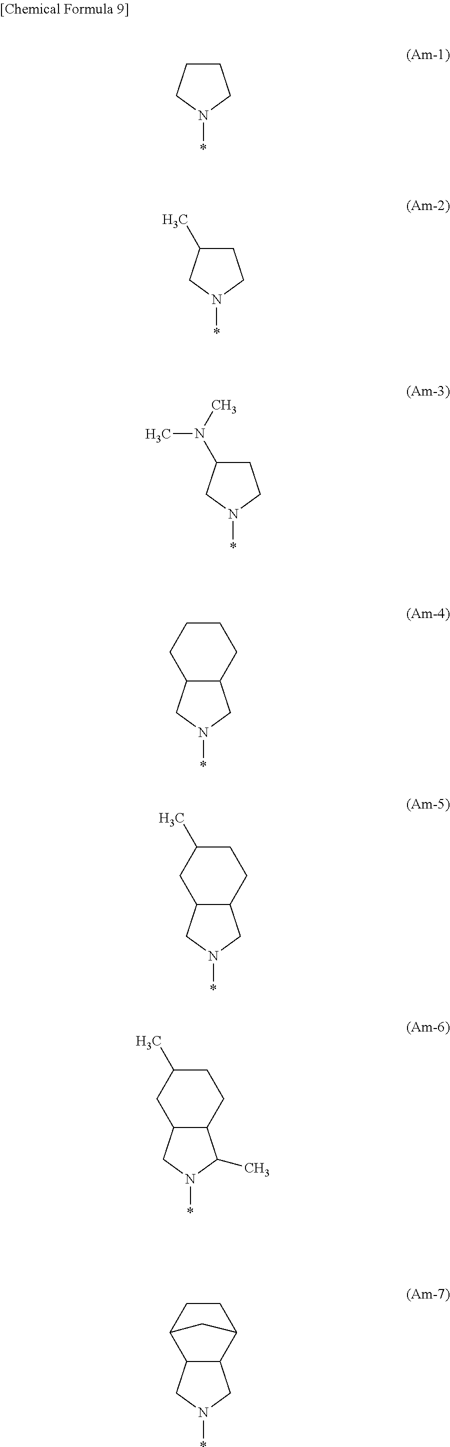

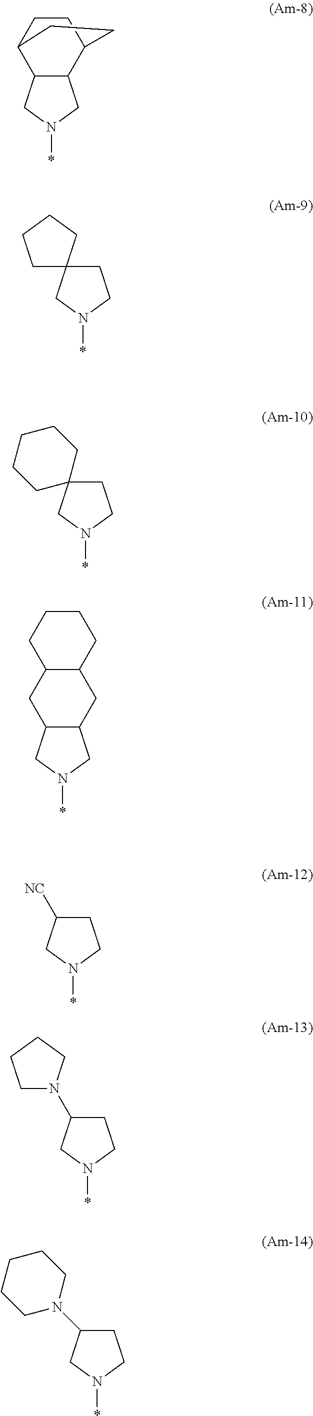

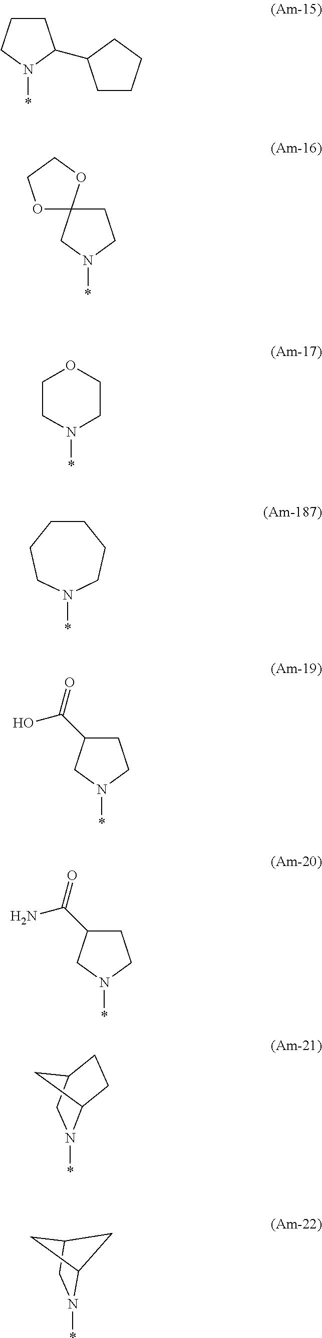

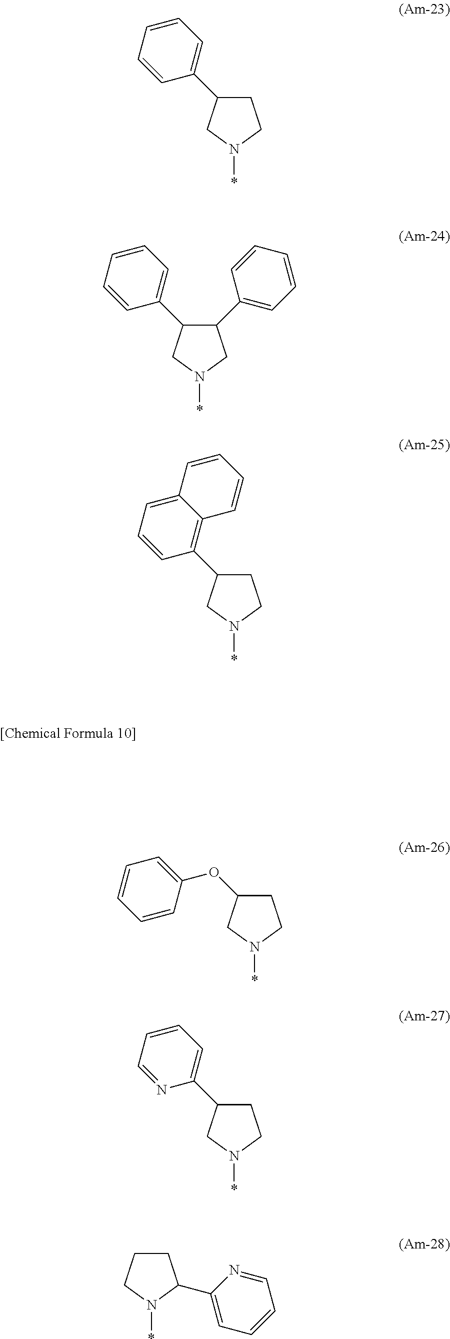

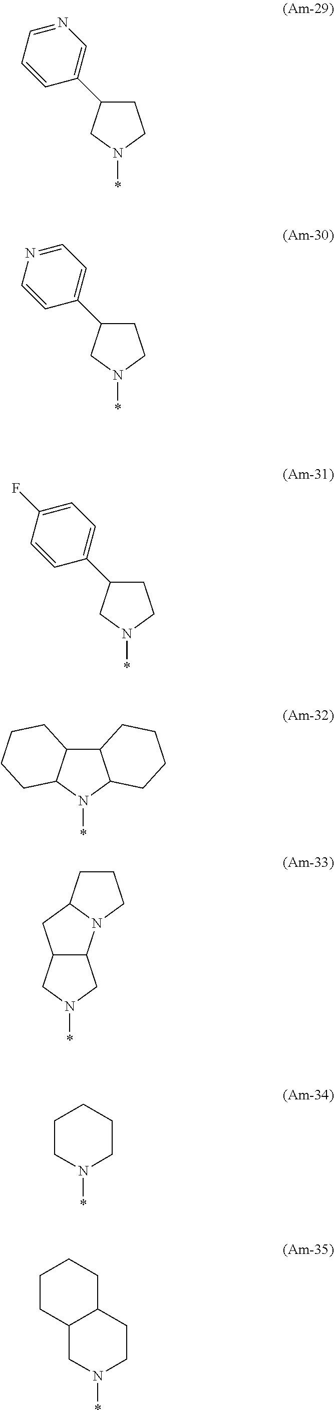

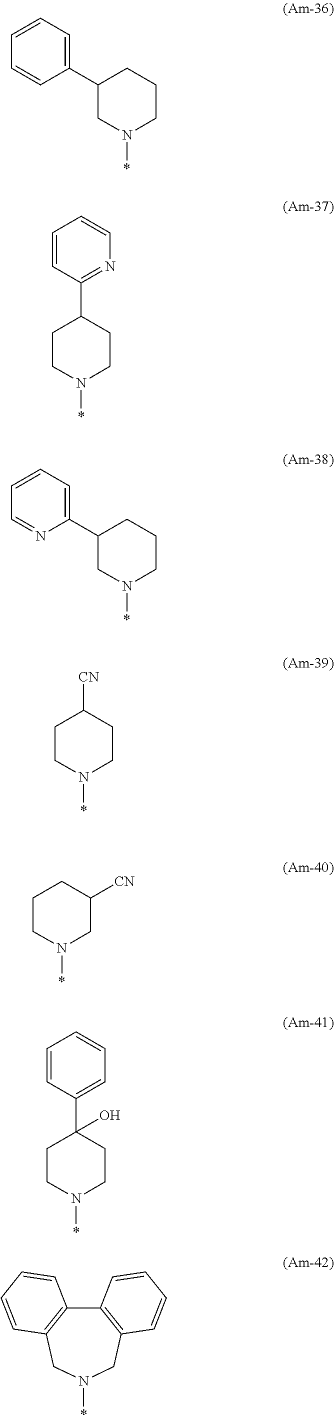

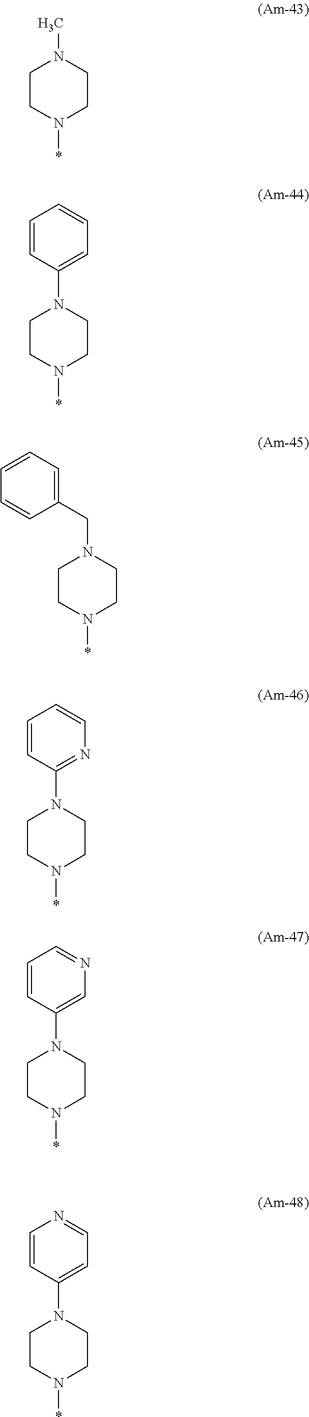

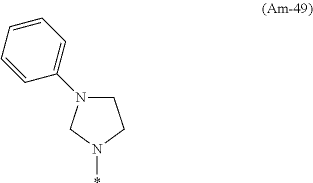

Examples of the organic compounds represented by General Formulas (G1) and (G2) above include organic compounds represented by Structural Formulas (100) to (117) below.

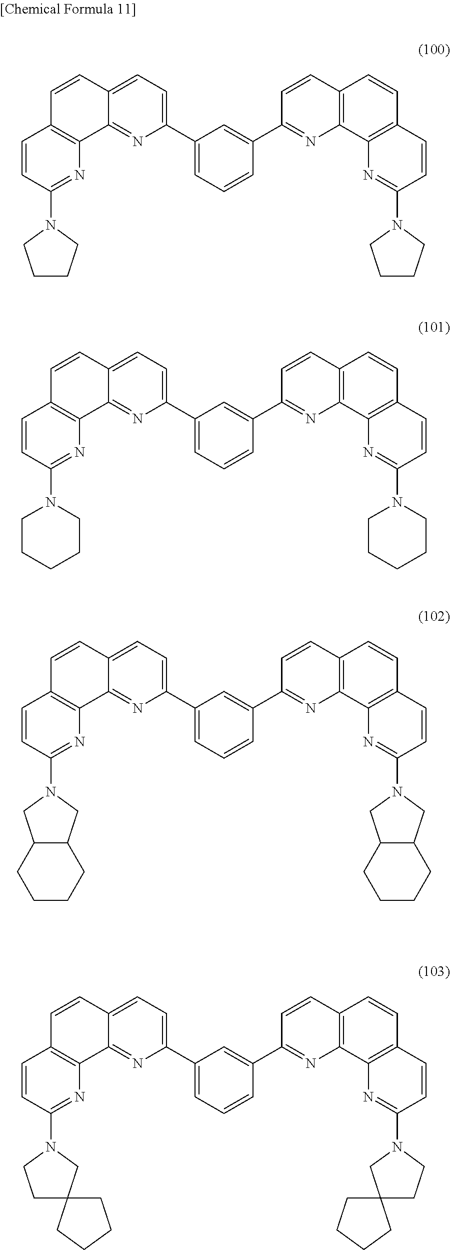

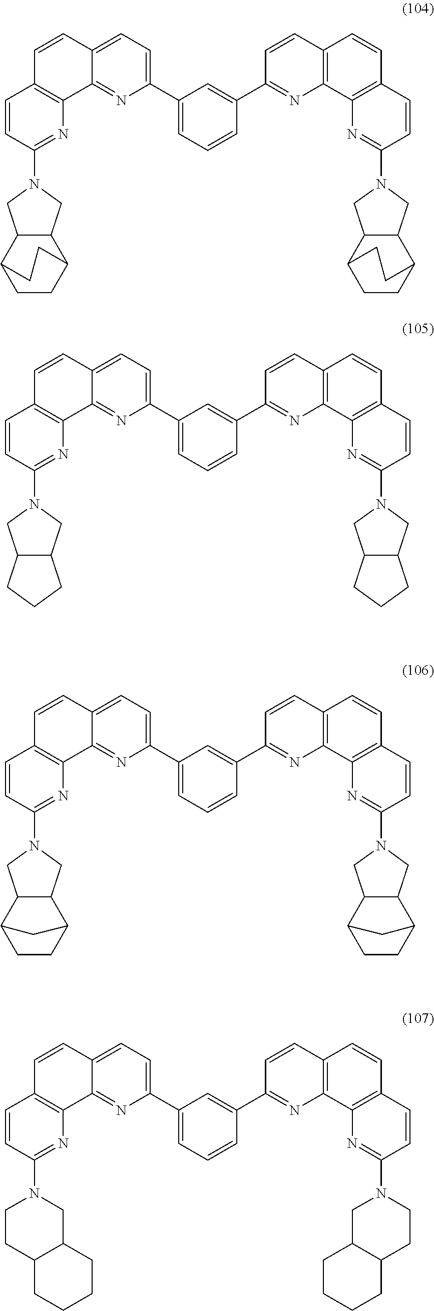

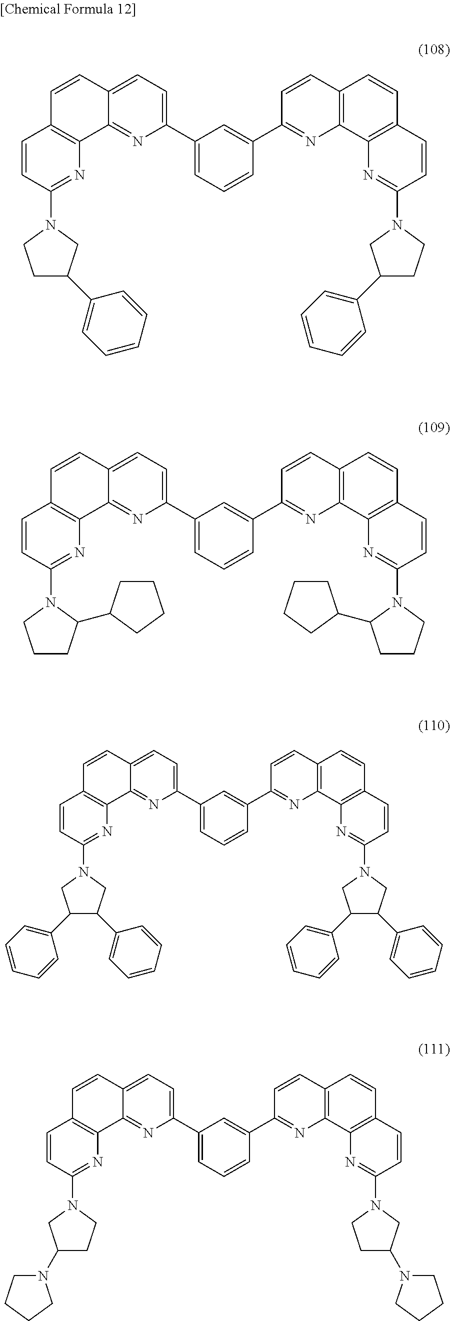

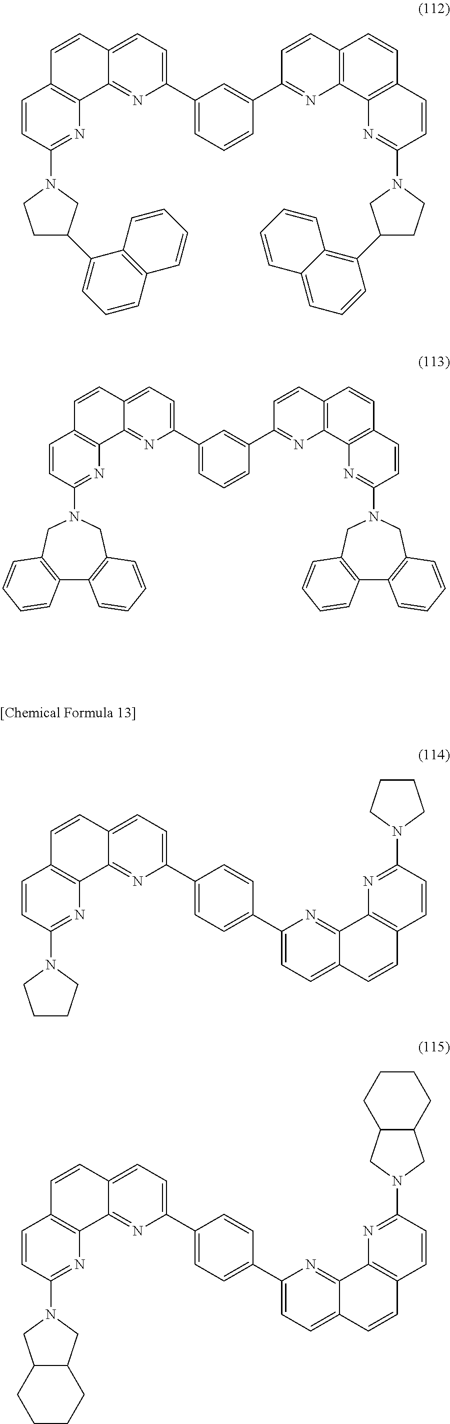

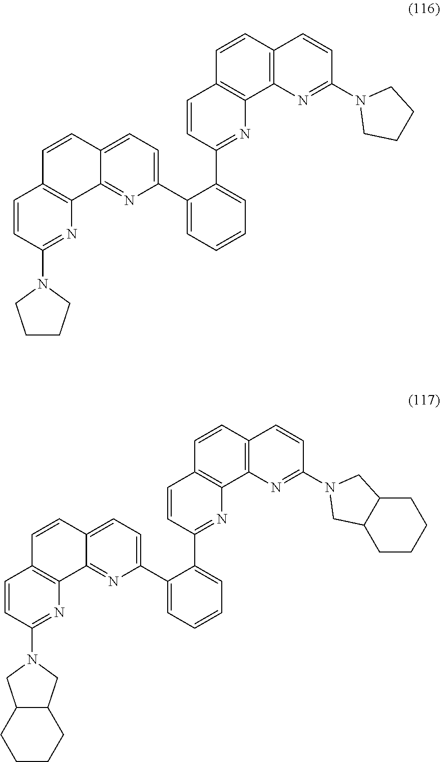

The organic compound of one embodiment of the present invention with any of the above structures can have favorable electron-transport property. The organic compound of one embodiment of the present invention can interact with a metal and thus can be suitably used as a material for an n-type layer of an intermediate layer in a tandem light-emitting device. Since the organic compound of one embodiment of the present invention has high heat resistance, a highly reliable light-emitting device that is highly resistant to high-temperature processing or preservation or driving at high temperatures can be provided. Since the organic compound of one embodiment of the present invention has a strong interaction with a metal, the organic compound is less likely to be affected by exposure to the air in the manufacturing process of the light-emitting device; therefore, the use of the organic compound for a tandem light-emitting device obtained through processing by a photolithography method enables a display device to have favorable characteristics and high resolution.

Next, a method for synthesizing the organic compound represented by General Formula (G1) below, which is an example of the organic compound of one embodiment of the present invention, is described with reference to General Formula (G1-1). Note that the method for synthesizing the organic compound represented by General Formula (G1) can employ a variety of reactions and is not limited to the following synthesis method.

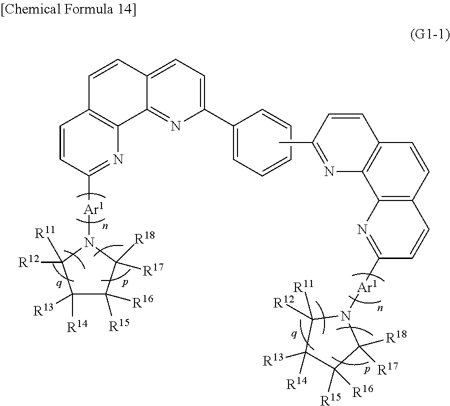

In General Formula (G1-1) above, R¹¹ to R¹⁸ each independently represent any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group, and p and q each independently represent 0 to 3. Any two of R¹¹ to R¹⁸ that are bonded to different carbon atoms may be bonded to each other to form a ring. An aliphatic cyclic amino group represented by General Formula (g1) above may be condensed with an aromatic ring having 6 to 10 carbon atoms. Ar¹ represents any one of a substituted or unsubstituted alkylene group having 1 to 3 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms, and n represents an integer of 0 to 3. When n is 2 or more, a plurality of Ar¹s may be the same or different from each other.

The organic compound represented by General Formula (G1-1) above can be synthesized by a simple synthesis scheme such as Synthesis Scheme (A-1) below.

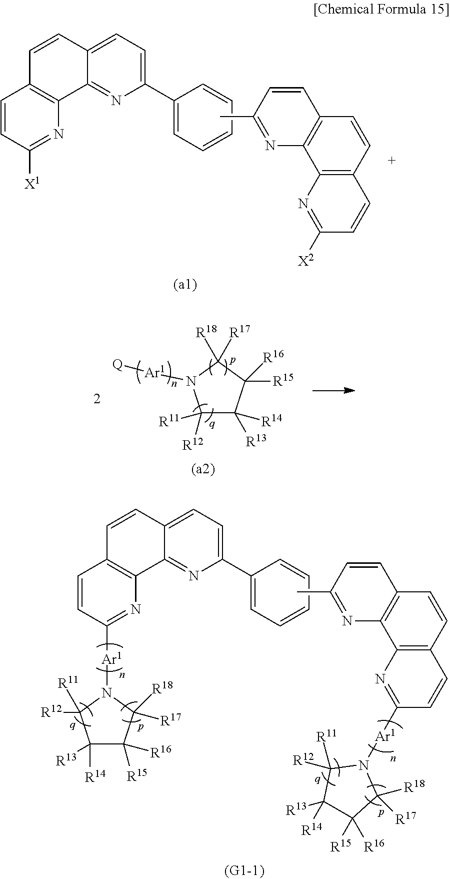

In the aliphatic cyclic amine derivative (a2), Q represents hydrogen when n is 0 and represents a boronyl group (—B(OH)₂) when n is 1 or more. R¹¹ to R¹⁸, p and q are similar to those in General Formula (g1). In the case where Q represents a boronyl group, a boronic ester, a cyclic-triolborate salt, or the like may be used.

In Synthesis Scheme (A-1), when n in the aliphatic cyclic amine derivative (a2) is 0, the compound (a1) and the aliphatic cyclic amine derivative (a2) undergo a nucleophilic substitution reaction to give the organic compound represented by General Formula (G1-1).

Examples of the base that can be used in the nucleophilic substitution reaction represented by Synthesis Scheme (A-1) above include an organic base such as 1,8-diazabicyclo[5.4.0]-7-undecene (abbreviation: DBU (registered trademark)), triethylamine, sodium-tert-butoxide, or potassium-tert-butoxide; an inorganic base such as potassium carbonate, cesium carbonate, sodium carbonate, sodium hydrogen carbonate, potassium acetate, sodium acetate, tripotassium phosphate, or trisodium phosphate.

Examples of the solvent that can be used in the nucleophilic substitution reaction represented by Synthesis Scheme (A-1) above include N-methyl-2-pyrrolidone, N,N-dimethylformamide, toluene, tetrahydrofuran, dioxane, and ethanol. However, the solvent that can be used is not limited to these solvents. In the case where an organic base is used, the organic base may be used as both the base and the solvent.

The reaction employed in Synthesis Scheme (A-1) above is not limited to the nucleophilic substitution reaction; a Buchwald-Hartwig reaction, a coupling reaction using copper or a copper compound, or the like can be used.

In Synthesis Scheme (A-1), when n in the aliphatic cyclic amine derivative (a2) is 1 or more, the compound (a1) and the aliphatic cyclic amine derivative (a2) are coupled by the Suzuki-Miyaura reaction to give the organic compound represented by General Formula (G1-1).

When the Suzuki-Miyaura reaction is performed for Synthetic Scheme (A-1) above, examples of the palladium catalyst that can be used include palladium(II) acetate, tetrakis(triphenylphosphine)palladium(0), bis(triphenylphosphine)palladium(II) dichloride, and tris(dibenzylideneacetone)dipalladium(0).

Examples of a ligand of the above palladium catalyst include 2-dicyclohexylphosphino-2′,6′-dimethoxybiphenyl, di(1-adamantyl)-N-butylphosphine, (±)-2,2′-bis(diphenylphosphino)-1,1′-binaphthyl, tri(ortho-tolyl)phosphine, triphenylphosphine, 4,5-bis(diphenylphosphino)-9,9-dimethylxanthene (abbreviation: Xantphos), and tricyclohexylphosphine.

In the case where the Suzuki-Miyaura reaction is performed for Synthesis Scheme (A-1) above, examples of the base that can be used include an organic base such as sodium-tert-butoxide and potassium-tert-butoxide, and an inorganic base such as potassium carbonate and sodium carbonate.

In the case where the Suzuki-Miyaura reaction is performed for Synthesis Scheme (A-1) above, examples of the solvent that can be used include toluene, xylene, mesitylene, benzene, tetrahydrofuran, and dioxane. However, the solvent that can be used is not limited to these solvents.

The reaction employed in the above synthesis scheme (A-1) is not limited to the Suzuki-Miyaura reaction. A Migita-Kosugi-Stille coupling reaction using an organotin compound, a coupling reaction using a Grignard reagent, or the like can be employed.

The organic compound of one embodiment of the present invention can be synthesized in the above manner, but the present invention is not limited thereto, and any other synthesis method may be employed.

This embodiment can be freely combined with any of the other embodiments and the examples.

### Embodiment 2

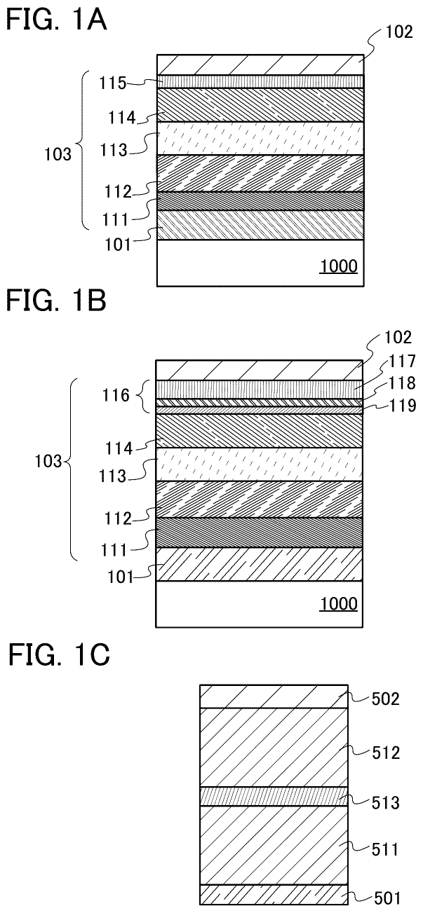

In this embodiment, a light-emitting device of one embodiment of the present invention will be described in detail. FIG. 1A illustrates a light-emitting device of one embodiment of the present invention. The light-emitting device of one embodiment of the present invention includes an organic compound layer 103 between a first electrode 101 formed over an insulating layer 1000 and a second electrode 102 facing the first electrode. The organic compound layer 103 includes at least a light-emitting layer 113, and may further include another functional layer. In the examples illustrated in FIGS. 1A and 1B, a hole-injection layer 111, a hole-transport layer 112, an electron-transport layer 114, and an electron-injection layer 115 (a charge-generation layer 116) are provided, and an exciton-blocking layer, an intermediate layer, or the like may be provided. In some cases, a layer in the hole-transport layer 112 that is in contact with the light-emitting layer 113 is specifically referred to as an electron-blocking layer, and a layer in the electron-transport layer 114 that is in contact with the light-emitting layer is specifically referred to as a hole-blocking layer. In this embodiment, the case where the first electrode 101 and the second electrode 102 respectively function as an anode and a cathode is described as an example; however, the first electrode 101 and the second electrode 102 may respectively function as a cathode and an anode.

Note that in the light-emitting device of one embodiment of the present invention, the organic compound layer 103 includes the organic compound represented by General Formula (G1) or (G2) in Embodiment 1. Since the organic compound represented by General Formula (G1) or (G2) has an electron-transport property, the organic compound is preferably included in the electron-transport layer 114, the electron-injection layer 115, the charge-generation layer 116, the hole-blocking layer, the light-emitting layer 113, the intermediate layer, or the like in the light-emitting device illustrated in FIGS. 1A and 1B.

In particular, the organic compound represented by General Formula (G1) or (G2) forms a coordinate bond with a metal or a metal compound and improves an electron-doner property of a metal and thus is preferably used, together with the metal or the metal compound, in the electron-injection layer 115, the charge-generation layer 116, and the intermediate layer in the light-emitting device illustrated in FIGS. 1A and 1B.

The electron-injection layer 115, the charge-generation layer 116, and the intermediate layer that include the organic compound which is represented by General Formula (G1) or (G2) disclosed in Embodiment 1 enable improvement of the electron-donor property of the metal or the metal compound, when the organic compound coordinates to a metal or a metal compound. This can inhibit impairment of the functions of the electron-injection layer 115, the charge-generation layer 116, and the intermediate layer even when the organic compound layer 103 is exposed to an air atmosphere, whereby an increase in driving voltage can be inhibited and a light-emitting device with favorable characteristics can be provided.

That is, the light-emitting device, which includes the electron-injection layer 115, the charge-generation layer 116, and the intermediate layer that include the metal or the metal compound and the organic compound represented by General Formula (G1) or (G2) disclosed in Embodiment 1, can have favorable characteristics without a significant increase in driving voltage even when subjected to processing by a photolithography method involving an air exposure step.

Since the organic compound represented by General Formula (G1) or (G2) disclosed in Embodiment 1 has high heat resistance, a highly reliable light-emitting device that is highly resistant to high-temperature processing or preservation or driving at high temperatures can be provided.

Furthermore, since the organic compound of one embodiment of the present invention has a strong interaction with a metal, which is less likely to be affected by exposure to the air in the manufacturing process of the light-emitting device, the use of the organic compound for a tandem light-emitting device obtained through processing by a photolithography method enables a display device to have favorable characteristics and high resolution.

This embodiment describes an example in which the first electrode 101 includes an anode, the second electrode 102 includes a cathode, and the first electrode 101 is formed on the insulating layer 1000 side; however, a structure in which the second electrode 102 is formed on the insulating layer 1000 side, what is called an inversely stacked structure, may be employed. In this case, the light-emitting device has a stacked-layer structure in which the second electrode 102, the electron-injection layer 115, (the electron-transport layer 114), the light-emitting layer 113, (the hole-transport layer 112, the hole-injection layer 111), and the first electrode 101 are stacked in this order from the insulating layer 1000 side. In the case of such a light-emitting device having an inversely stacked structure, the relatively stable hole-injection layer 111 serves as a surface; thus, the light-emitting device can have higher reliability.

The first electrode 101 and the second electrode 102 may each have a single-layer structure or a stacked-layer structure. In the case of the stacked-layer structure, a layer in contact with the organic compound layer 103 serves as an anode or a cathode. In the case where the electrodes each have the stacked-layer structure, there is no limitation on work functions of materials for layers other than the layer in contact with the organic compound layer 103, and the materials can be selected in accordance with required properties such as a resistance value, processing easiness, reflectivity, light-transmitting property, and stability.

The anode is preferably formed using any of metals, alloys, and conductive compounds with a high work function (specifically, higher than or equal to 4.0 eV), mixtures thereof, and the like. Specific examples include indium oxide-tin oxide (ITO: indium tin oxide), indium oxide-tin oxide including silicon or silicon oxide (ITSO: indium tin silicon oxide), indium oxide-zinc oxide, and indium oxide including tungsten oxide and zinc oxide (IWZO). Such conductive metal oxide films are usually formed by a sputtering method, but may be formed by application of a sol-gel method or the like. For example, a film of indium oxide-zinc oxide is formed by a sputtering method using a target in which 1 wt % to 20 wt % zinc oxide is added to indium oxide. Furthermore, a film of indium oxide including tungsten oxide and zinc oxide (IWZO) can be formed by a sputtering method using a target in which 0.5 wt % to 5 wt % tungsten oxide and 0.1 wt % to 1 wt % zinc oxide are added to indium oxide. Alternatively, gold (Au), platinum (Pt), nickel (Ni), tungsten (W), chromium (Cr), molybdenum (Mo), iron (Fe), cobalt (Co), copper (Cu), palladium (Pd), titanium, (Ti), aluminum (Al), nitride of a metal material (e.g., titanium nitride), or the like can be used for the anode. The anode may be a stack of layers formed of any of these materials. For example, a film in which Al, Ti, and ITSO are stacked in this order over Ti is preferable because the film has high efficiency owing to high reflectivity and enables high resolution of several thousand ppi. Graphene can also be used for the anode. When a composite material that can be included in the hole-injection layer 111, which is described later, is used for a layer (typically, the hole-injection layer) in contact with the anode, an electrode material can be selected regardless of its work function.

The hole-injection layer 111 is provided in contact with the anode and has a function of facilitating injection of holes to the organic compound layer 103. The hole-injection layer 111 can be formed using a phthalocyanine-based compound or complex compound such as phthalocyanine (abbreviation: H₂Pc) or copper phthalocyanine (abbreviation: CuPc), an aromatic amine compound such as 4,4′-bis[N-(4-diphenylaminophenyl)-N-phenylamino]biphenyl (abbreviation: DPAB) or 4,4′-bis(N-{4-[N′-(3-methylphenyl)-N′-phenylamino]phenyl}-N-phenylamino)biphenyl (abbreviation: DNTPD), or a high molecular compound such as poly(3,4-ethylenedioxythiophene)/polystyrenesulfonic acid (abbreviation: PEDOT/PSS), for example.

The hole-injection layer 111 may be formed using a substance having an electron-acceptor property. Examples of the substance having an acceptor property include organic compounds having an electron-withdrawing group (a halogen group, a cyano group, or the like), such as 7,7,8,8-tetracyano-2,3,5,6-tetrafluoroquinodimethane (abbreviation: F4-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylen-1,3,4,5,6,8,9,10-octafluoro-7H-pyren-2-ylidene)malononitrile. A compound in which electron-withdrawing groups are bonded to a condensed aromatic ring having a plurality of heteroatoms, such as HAT-CN, is particularly preferable because it is thermally stable. A [3]radialene derivative having an electron-withdrawing group (in particular, a cyano group, a halogen group such as a fluoro group, or the like) has a significantly high electron-acceptor property and thus is preferable. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, a transition metal oxide such as molybdenum oxide, vanadium oxide, ruthenium oxide, tungsten oxide, or manganese oxide can be used, other than the above-described organic compounds. Alternatively, the hole-injection layer 111 can be formed using a phthalocyanine-based compound or a complex compound such as phthalocyanine (abbreviation: H₂Pc) or copper phthalocyanine (abbreviation: CuPc), an aromatic amine compound such as 4,4′-bis[N-(4-diphenylaminophenyl)-N-phenylamino]biphenyl (abbreviation: DPAB) or 4,4′-bis(N-{4-[N′-(3-methylphenyl)-N′-phenylamino]phenyl}-N-phenylamino)biphenyl (abbreviation: DNTPD), or a high molecular compound such as poly(3,4-ethylenedioxythiophene)/poly(styrenesulfonic acid) (abbreviation: PEDOT/PSS), for example. The substance having an acceptor property can extract electrons from an adjacent hole-transport layer (or hole-transport material) by application of an electric field.

The hole-injection layer 111 is preferably formed using a composite material including any of the aforementioned materials having an acceptor property and a substance having a hole-transport property.

As the substance having a hole-transport property used in the composite material, any of a variety of organic compounds such as aromatic amine compounds, heteroaromatic compounds, aromatic hydrocarbons, and high molecular compounds (e.g., oligomers, dendrimers, and polymers) can be used. Note that the substance having a hole-transport property used in the composite material preferably has a hole mobility higher than or equal to 1×10⁻⁶ cm²/Vs. The substance having a hole-transport property used in the composite material is preferably a compound having a condensed aromatic hydrocarbon ring or a π-electron rich heteroaromatic ring. As the condensed aromatic hydrocarbon ring, an anthracene ring, a naphthalene ring, or the like is preferable. As the π-electron rich heteroaromatic ring, a condensed aromatic ring having at least one of a pyrrole skeleton, a furan skeleton, and a thiophene skeleton is preferable; specifically, a carbazole ring, a dibenzothiophene ring, or a ring in which an aromatic ring or a heteroaromatic ring is condensed to a carbazole ring or a dibenzothiophene ring is preferable.

Such a substance having a hole-transport property further preferably has at least one of a carbazole skeleton, a dibenzofuran skeleton, a dibenzothiophene skeleton, and an anthracene skeleton. In particular, an aromatic amine having a substituent that includes a dibenzofuran ring or a dibenzothiophene ring, an aromatic monoamine that has a naphthalene ring, or an aromatic monoamine in which a 9-fluorenyl group is bonded to nitrogen of an amine through an arylene group may be used. Note that the substance having a hole-transport property preferably has an N,N-bis(4-biphenyl)amino group, enabling manufacturing a light-emitting device with a long lifetime.

Specific examples of the substance having a hole-transport property include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAPNB), 4-[4-(2-naphthyl)phenyl]-4′,4″-diphenyltriphenylamine (abbreviation: BBAβNBi), 4,4′-diphenyl-4″-([2,1′-binaphthyl]-6-yl)triphenylamine (abbreviation: BBAαNβNB), 4,4′-diphenyl-4″-([2,1′-binaphthyl]-7-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-([2,2′-binaphthyl]-6-yl)triphenylamine (abbreviation: BBA(βN2)B), 4,4′-diphenyl-4″-([2,2′-binaphthyl]-7-yl)triphenylamine (abbreviation: BBA(βN2)B-03), 4,4′-diphenyl-4″-([1,2′-binaphthyl]-4-yl)triphenylamine (abbreviation: BBAβNαNB), 4,4′-diphenyl-4″-([1,2′-binaphthyl]-5-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-N-[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, N,N-bis(9,9-dimethyl-9H-fluoren-2-yl)-9,9′-spirobi-9H-fluoren-1-amine, 9-[3-(triphenylsilyl)phenyl]-3,9′-bi-9H-carbazole (abbreviation: PSiCzCz), and 9′-[3-(triphenylsilyl)phenyl]-9′H-9,3′:6′,9″-tercarbazole (abbreviation: PSiCzGI).

Moreover, examples of other aromatic amine compounds that can be used as the substance having a hole-transport property include N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), 4,4′-bis(N-{4-[N′-(3-methylphenyl)-N-phenylamino]phenyl}-N-phenylamino)biphenyl (abbreviation: DNTPD), and 1,3,5-tris[N-(4-diphenylaminophenyl)-N-phenylamino]benzene (abbreviation: DPA3B).

The hole-injection layer 111 can improve the hole-injection property, which allows a light-emitting device to be driven at a low voltage.

Among substances having an acceptor property, an organic compound having an acceptor property is easy to use because it is easily deposited by evaporation.

The hole-transport layer 112 is formed using a substance having a hole-transport property. The substance having a hole-transport property preferably has a hole mobility higher than or equal to 1×10⁻⁶ cm²/Vs.

Although the hole-transport layer 112 may have a single-layer structure or a stacked-layer structure, the layer in the hole-transport layer 112 in contact with the light-emitting layer 113 has the structure described in Embodiment 1. Note that the repeated description of the structure is omitted.

In the case where the hole-transport layer 112 has a stacked-layer structure, the structure described in Embodiment 1 may not be necessarily employed for layers other than the layer in contact with the light-emitting layer 113. In the hole-transport layer having a stacked-layer structure, the layers other than the layer in contact with the light-emitting layer 113 are formed to include a substance having a hole-transport property. The substance having a hole-transport property preferably has a hole mobility higher than or equal to 1×10⁻⁶ cm²/Vs.

Examples of the above substance having a hole-transport property include the following compounds: compounds having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), 9-(2-naphthyl)-9′-phenyl-3,3′-bi-9H-carbazole (abbreviation: βNCCP), 9-(3-biphenyl)-9′-(2-naphthyl)-3,3′-bi-9H-carbazole (abbreviation: βNCCmBP), 9-(4-biphenyl)-9′-(2-naphthyl)-3,3′-bi-9H-carbazole (abbreviation: βNCCBP), 9,9′-di-2-naphthyl-3,3′-9H,9′H-bicarbazole (abbreviation: BisβNCz), 9-(2-naphthyl)-9′-[1,1′:4′,1″-terphenyl]-3-yl-3,3′-9H,9′H-bicarbazole, 9-(2-naphthyl)-9′-[1,1′:3′,1″-terphenyl]-3-yl-3,3′-9H,9′H-bicarbazole, 9-(2-naphthyl)-9′-[1,1′:3′,1″-terphenyl]-5′-yl-3,3′-9H,9′H-bicarbazole, 9-(2-naphthyl)-9′-[1,1′:4′,1″-terphenyl]-4-yl-3,3′-9H,9′H-bicarbazole, 9-(2-naphthyl)-9′-[1,1′:3′,1″-terphenyl]-4-yl-3,3′-9H,9′H-bicarbazole, 9-(2-naphthyl)-9′-(triphenylen-2-yl)-3,3′-9H,9′H-bicarbazole, 9-phenyl-9′-(triphenylen-2-yl)-3,3′-9H,9′H-bicarbazole (abbreviation: PCCzTp), 9,9′-bis(triphenylen-2-yl)-3,3′-9H,9′H-bicarbazole, 9-(4-biphenyl)-9′-(triphenylen-2-yl)-3,3′-9H,9′H-bicarbazole, 9-(triphenylen-2-yl)-9′-[1,1′:3′,1″-terphenyl]-4-yl-3,3′-9H,9′H-bicarbazole, N,N-bis(9,9-dimethyl-9H-fluoren-2-yl)-9,9′-spirobi-9H-fluoren-1-amine, and 9-[3-(triphenylsilyl)phenyl]-3,9′-bi-9H-carbazole (abbreviation: PSiCzCz); compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above substances, the compound having an aromatic amine skeleton or the compound having a carbazole skeleton is preferable because the compound is highly reliable and has a high hole-transport property to contribute to a reduction in driving voltage. Any of the organic compounds given as examples of the substance having a hole-transport property used in the composite material for the hole-injection layer 111 can also be suitably used as the material included in the hole-transport layer 112. An organic compound having an amine skeleton and a fluorene skeleton is further preferably used. The organic compound having an amine skeleton and a fluorene skeleton is preferable because its high reliability and high hole-transport property enable power consumption of a light-emitting device to be reduced.

The light-emitting layer 113 includes an emission center substance. In addition, the light-emitting layer 113 preferably includes a host material.

The emission center substance may be a fluorescent substance, a phosphorescent substance, a substance exhibiting thermally activated delayed fluorescence (TADF), or any other light-emitting substance.

Examples of the material that can be used as a fluorescent substance in the light-emitting layer are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N′-bis[4-(9-phenyl-9H-fluoren-9-yl)phenyl]pyrene-1,6-diamine (abbreviation: 1,6FLPAPrn), N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N′-(2-tert-butylanthracene-9,10-diyldi-4,1-phenylene)bis(N,N′,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N′-diphenyl-N,N′-(1,6-pyrene-diyl)bis[(6-phenylbenzo[b]naphtho[1,2-d]furan)-8-amine](abbreviation: 1,6BnfAPrn-03), N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPrn, 1,6mMemFLPAPm, and 1,6BnfAPrn-03 are particularly preferable because of their high hole-trapping properties, high emission efficiency, or high reliability.

A condensed heteroaromatic compound including nitrogen and boron, especially a compound having a diaza-boranaphtho-anthracene skeleton, emits high color purity blue light with a narrow emission spectrum and can thus be suitably used. Examples of the compound include 5,9-diphenyl-5H,9H-[1,4]benzazaborino[2,3,4-ki]phenazaborine (abbreviation: DABNA-1), 9-(biphenyl-3-yl)-N,N,5,11-tetraphenyl-5H,9H-[1,4]benzazaborino[2,3,4-kl]phenazaborin-3-amine (abbreviation: DABNA-2), 2,12-di(tert-butyl)-5,9-di(4-tert-butylphenyl)-N,N-diphenyl-5H,9H-[1,4]benzazaborino[2,3,4-kl]phenazaborin-7-amine (abbreviation: DPhA-tBu4DABNA), 2,12-di(tert-butyl)-N,N,5,9-tetra(4-tert-butylphenyl)-5H,9H-[1,4]benzazaborino[2,3,4-kl]phenazaborin-7-amine (abbreviation: tBuDPhA-tBu4DABNA), 2,12-di(tert-butyl)-5,9-di(4-tert-butylphenyl)-7-methyl-5H,9H-[1,4]benzazaborino[2,3,4-kl]phenazaborine (abbreviation: Me-tBu4DABNA), N⁷,N⁷,N¹³,N¹³,5,9,11,15-octaphenyl-5H,9H,11H,15H-[1,4]benzazaborino[2,3,4-kl][1,4]benzazaborino[4′,3′,2′:4,5][1,4]benzazaborino[3,2-b]phenazaborine-7,13-diamine (abbreviation: v-DABNA), and 2-(4-tert-butylphenyl)benz[5,6]indolo[3,2,1-jk]benzo[b]carbazole (abbreviation: tBuPBibc).

Besides the above compounds, a compound having an indole skeleton, such as 9,10,11-tris[3,6-bis(1,1-dimethylethyl)-9H-carbazolyl-9-yl]-2,5,15,18-tetrakis(1,1-dimethylethyl)indolo[3,2,1-de]indolo[3′,2′,1′:8,1][1,4]benzazaborino[2,3,4-kl]phenazaborine (abbreviation: BBCz-G) or 9,11-bis[3,6-bis(1,1-dimethylethyl)-9H-carbazolyl-9-yl]-2,5,15,18-tetrakis(1,1-dimethylethyl)indolo[3,2,1-de]indolo[3′,2′,1′:8,1][1,4]benzazaborino[2,3,4-kl]phenazaborine (abbreviation: BBCz-Y), can be suitably used.

A preferable phosphorescent substance that can be used as the emission center substance in the light-emitting layer is a metal complex, in particular, an iridium complex or a platinum complex, examples of which are as follows.

The examples include organometallic iridium complexes having a 4H-triazole skeleton, such as tris{2-[5-(2-methylphenyl)-4-(2,6-dimethylphenyl)-4H-1,2,4-triazol-3-yl-KN²]phenyl-KC}iridium(III) (abbreviation: [Ir(mpptz-dmp)₃]) and tris(5-methyl-3,4-diphenyl-4H-1,2,4-triazolato)iridium(III) (abbreviation: [Ir(Mptz)₃]); organometallic iridium complexes having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic iridium complexes having an imidazole skeleton, such as fac-tris[1-(2,6-diisopropylphenyl)-2-phenyl-1H-imidazole]iridium(III) (abbreviation: [Ir(iPrpim)₃]), tris[3-(2,6-dimethylphenyl)-7-methylimidazo[1,2-f]phenanthridinato]iridium(III) (abbreviation: [Ir(dmpimpt-Me)₃]), and tris(2-{1-[2,6-bis(1-methylethyl)phenyl]-1H-imidazol-2-yl-κN³}-4-cyanophenyl-κC)iridium(III) (abbreviation: CNImIr); organometallic complexes having a benzimidazolidene skeleton, such as tris[(6-tert-butyl-3-phenyl-2H-imidazo[4,5-b]pyrazin-1-yl-κC²)phenyl-κC]iridium(III) (abbreviation: [Ir(cb)₃]); organometallic iridium complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIracac); and platinum complexes such as (2-{3-[3-(3,5-di-tert-butylphenyl)benzimidazol-1-yl-2-ylidene-κC²]phenoxy-κC²}-9-(4-tert-butyl-2-pyridinyl-κN)carbazole-2,1-diyl-κC¹)platinum(II) (abbreviation: PtON-TBBI). These compounds emit phosphorescent light with a blue hue and have an emission peak in the wavelength range from 450 nm to 520 nm. A compound obtained by substituting deuterium for part of hydrogen in any of these compounds can also be used.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)), {2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d6)₂(mbfpypy-iPr-d₄)), [2-(methyl-d₃)-8-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d₃)), [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)), [2-(4-d₃-methyl-5-phenyl-2-pyridinyl-κN²)phenyl-κC]bis[2-(5-d₃-methyl-2-pyridinyl-κN²)phenyl-κC]iridium(III) (abbreviation: [Ir(5mppy-d₃)₂(mdppy-d₃)]), [2-methyl-8-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy)]), [2-(4-methyl-5-phenyl-2-pyridinyl-κN)phenyl-κC]bis[2-(2-pyridinyl-κN)phenyl-κC]iridium (abbreviation: [Ir(ppy)₂(mdppy)]), and tris{2-[5-(methyl-d₃)-4-phenyl-2-pyridinyl-κN]phenyl-κC}iridium(III) (abbreviation: Ir(5m4dppy-d₃)₃); organometallic platinum complexes such as (2-{1-(5-tert-butylbiphenyl-2-yl)-4-[3-tert-butyl-5-(4-phenyl-2-pyridinyl-κN)phenyl-κC⁶]-2-benzimidazolyl-κN³}-4,6-di-tert-butylphenolato-κO)platinum(II) (abbreviation: Pt(tBudppymmtBubiz-tBubp)) and [2-(4-(3,5-di-tert-butylphenyl)-6-{3-[4-(5′-tert-butyl[1,1′:3′,1″-terphenyl]-2′-yl)-2-pyridinyl-κN]phenyl-κC²}-2-pyridinyl-κN)phenolato-κO]platinum(II) (abbreviation: Pt(4tButpppypyp-mmtBup); and rare earth metal complexes such as tris(acetylacetonato)(monophenanthroline)terbium(III) (abbreviation: [Tb(acac)₃(Phen)]). These compounds mainly emit phosphorescent light with a green hue and have an emission peak in the wavelength range from 500 nm to 600 nm. Note that organometallic iridium complexes having a pyrimidine skeleton exhibit remarkably high reliability or emission efficiency and thus are particularly preferable. A compound obtained by substituting deuterium for part of hydrogen in any of these compounds can also be used.

Other examples include organometallic iridium complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); organometallic iridium complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes having a pyridine skeleton, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), (3,7-diethyl-4,6-nonanedionato-κO⁴, κO⁶)bis[2,4-dimethyl-6-[7-(1-methylethyl)-1-isoquinolinyl-κN]phenyl-κC]iridium(III), and (3,7-diethyl-4,6-nonanedionato-κO⁴,κO⁶)bis[2,4-dimethyl-6-[5-(1-methylethyl)-2-quinolinyl-κN]phenyl-κC]iridium(III); platinum complexes such as 2,3,7,8,12,13,17,18-octaethyl-21H,23H-porphyrinplatinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds emit phosphorescent light with a red hue and have an emission peak in the wavelength range from 600 nm to 700 nm. Furthermore, the organometallic iridium complexes having a pyrazine skeleton can provide red light emission with favorable chromaticity. A compound obtained by substituting deuterium for part of hydrogen in any of these compounds can also be used.

Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), palladium (Pd), or the like can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulas.

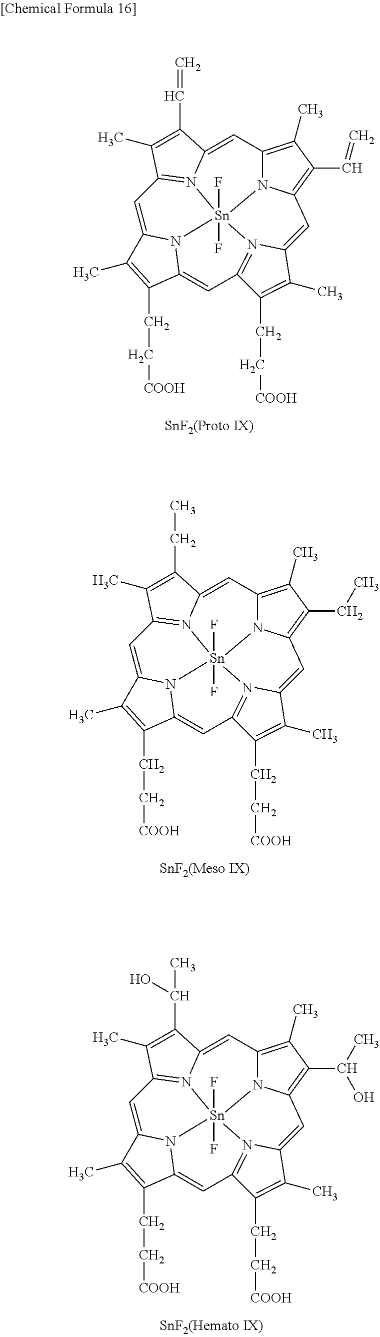

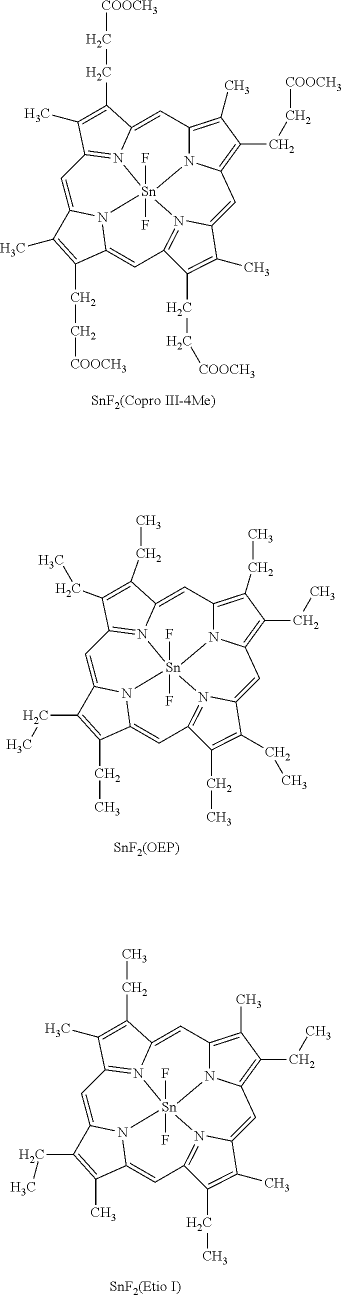

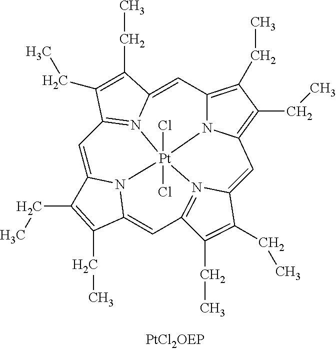

Alternatively, it is possible to use a heterocyclic compound having one or both of a π-electron rich heteroaromatic ring and a π-electron deficient heteroaromatic ring, which is represented by the following structural formulas, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA). Such a heterocyclic compound is preferable because of having high electron-transport and hole-transport properties owing to its π-electron rich heteroaromatic ring and its π-electron deficient heteroaromatic ring. Among skeletons having the π-electron deficient heteroaromatic ring, a pyridine skeleton, a diazine skeleton (a pyrimidine skeleton, a pyrazine skeleton, and a pyridazine skeleton), and a triazine skeleton are preferable because of their high stability and reliability. In particular, a benzofuropyrimidine skeleton, a benzothienopyrimidine skeleton, a benzofuropyrazine skeleton, and a benzothienopyrazine skeleton are preferable because of their high acceptor properties and high reliability. Among skeletons having the π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which a π-electron rich heteroaromatic ring is directly bonded to a π-electron deficient heteroaromatic ring is particularly preferable because the electron-donating property of the π-electron rich heteroaromatic ring and the electron-accepting property of the π-electron deficient heteroaromatic ring are both improved, the energy difference between the S₁ level and the T₁ level becomes small, and thus thermally activated delayed fluorescence can be obtained with high efficiency. Note that an aromatic ring to which an electron-withdrawing group such as a cyano group is bonded may be used instead of a π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton including boron such as phenylborane or boranthrene, an aromatic ring or a heteroaromatic ring having a nitrile group or a cyano group such as benzonitrile or cyanobenzene, a carbonyl skeleton such as benzophenone, a phosphine oxide skeleton, a sulfone skeleton, or the like can be used. As described above, a π-electron deficient skeleton or a π-electron rich skeleton can be used instead of at least one of a π-electron deficient heteroaromatic ring and a π-electron rich heteroaromatic ring.

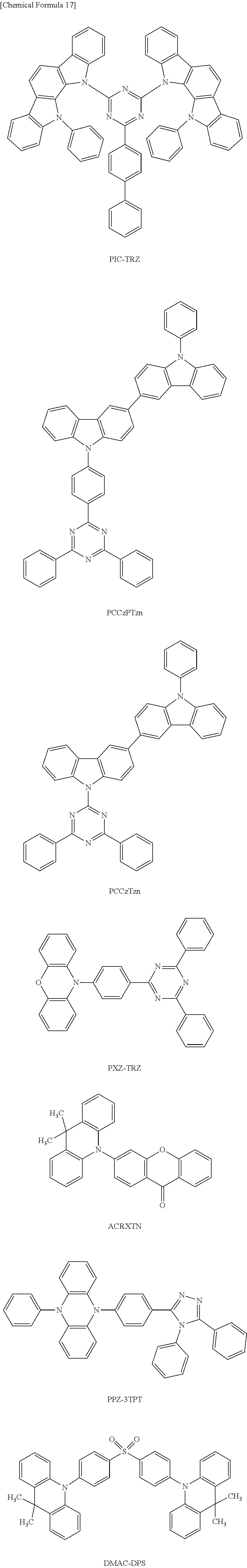

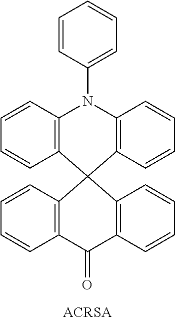 [SMILES: O=C1c2ccccc2C2(c3ccccc31)c1ccccc1N(c1ccccc1)c1ccccc12]

Alternatively, a TADF material whose singlet excited state and triplet excited state are in a thermal equilibrium state may be used. Since such a TADF material enables a short emission lifetime (excitation lifetime), the efficiency of a light-emitting device in a high-luminance region can be less likely to decrease. Specifically, a material having the following molecular structure can be used.

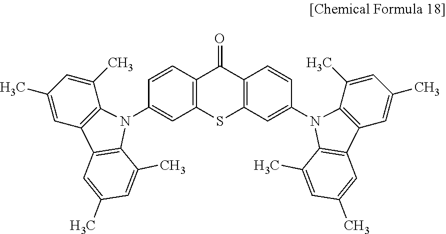

Note that a TADF material is a material having a small energy difference between the S₁ level and the T₁ level and a function of converting triplet excitation energy into singlet excitation energy by reverse intersystem crossing. Thus, a TADF material can upconvert triplet excitation energy into singlet excitation energy (i.e., reverse intersystem crossing) using a small amount of thermal energy and efficiently generate a singlet excited state. In addition, the triplet excitation energy can be converted into light emission.

An exciplex whose excited state is formed of two kinds of substances has an extremely small energy difference between the S₁ level and the T₁ level and functions as a TADF material capable of converting triplet excitation energy into singlet excitation energy.

A phosphorescence spectrum observed at low temperatures (e.g., 77 K to 10 K) can be used for an index of the T₁ level. When the level of energy with a wavelength of the line obtained by extrapolating a tangent to the fluorescence spectrum at a tail on the short wavelength side is the S₁ level and the level of energy with a wavelength of the line obtained by extrapolating a tangent to the phosphorescence spectrum at a tail on the short wavelength side is the T₁ level, the energy difference between the S₁ level and the T₁ level of the TADF material is preferably smaller than or equal to 0.3 eV, further preferably smaller than or equal to 0.2 eV.

When a TADF material is used as the light-emitting substance, the S₁ level of the host material is preferably higher than that of the TADF material. In addition, the T₁ level of the host material is preferably higher than that of the TADF material.

As the host material in the light-emitting layer, various carrier-transport materials such as materials having an electron-transport property and/or materials having a hole-transport property, and the TADF materials can be used.

The material having a hole-transport property is preferably an organic compound having an amine skeleton or a π-electron rich heteroaromatic ring skeleton, for example. As the π-electron rich heteroaromatic ring, a condensed aromatic ring having at least one of an acridine skeleton, a phenoxazine skeleton, a phenothiazine skeleton, a furan skeleton, a thiophene skeleton, and a pyrrole skeleton is preferable; specifically, a carbazole ring, a dibenzothiophene ring, or a ring in which an aromatic ring or a heteroaromatic ring is further condensed to a carbazole ring or a dibenzothiophene ring is preferable.

Such an organic compound having a hole-transport property further preferably has at least one of a carbazole skeleton, a dibenzofuran skeleton, a dibenzothiophene skeleton, and an anthracene skeleton. In particular, an aromatic amine having a substituent that includes a dibenzofuran ring or a dibenzothiophene ring, an aromatic monoamine that has a naphthalene ring, or an aromatic monoamine in which a 9-fluorenyl group is bonded to nitrogen of an amine through an arylene group may be used. Note that the organic compound having a hole-transport property preferably has an N,N-bis(4-biphenyl)amino group to enable manufacturing a light-emitting device with a long lifetime.

As such an organic compound, any of the following organic compounds is preferable, for example. Examples include compounds having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]-9,9′-spirobi[9H-fluoren]-2-amine (abbreviation: PCBASF); compounds having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 3,3′-bis(9-phenyl-9H-carbazole) (abbreviation: PCCP), 3,9-bis(9-phenyl-9H-carbazol-3-yl)-9H-carbazole (abbreviation: PCCzPC), 9-(biphenyl-4-yl)-9′-phenyl-3,3′-bi-9H-carbazole (abbreviation: PCCzBP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), 9-(2-naphthyl)-9′-phenyl-3,3′-bi-9H-carbazole (abbreviation: βNCCP), 9-(3-biphenyl)-9′-(2-naphthyl)-3,3′-bi-9H-carbazole (abbreviation: βNCCmBP), 9-(4-biphenyl)-9′-(2-naphthyl)-3,3′-bi-9H-carbazole (abbreviation: βNCCBP), 9,9′-di-2-naphthyl-3,3′-9H,9′H-bicarbazole (abbreviation: BisβNCz), 9-(2-naphthyl)-9′-[1,1′:4′,1″-terphenyl]-3-yl-3,3′-9H,9′H-bicarbazole, 9-(2-naphthyl)-9′-[1,1′:3′,1″-terphenyl]-3-yl-3,3′-9H,9′H-bicarbazole, 9-(2-naphthyl)-9′-[1,1′:3′,1″-terphenyl]-5′-yl-3,3′-9H,9′H-bicarbazole, 9-(2-naphthyl)-9′-[1,1′:4′,1″-terphenyl]-4-yl-3,3′-9H,9′H-bicarbazole, 9-(2-naphthyl)-9′-[1,1′:3′,1″-terphenyl]-4-yl-3,3′-9H,9′H-bicarbazole, 9-(2-naphthyl)-9′-(triphenylen-2-yl)-3,3′-9H,9′H-bicarbazole, 9-phenyl-9′-(triphenylen-2-yl)-3,3′-9H,9′H-bicarbazole (abbreviation: PCCzTp), 9,9′-bis(triphenylen-2-yl)-3,3′-9H,9′H-bicarbazole, 9-(4-biphenyl)-9′-(triphenylen-2-yl)-3,3′-9H,9′H-bicarbazole, 9-(triphenylen-2-yl)-9′-[1,1′:3′,1″-terphenyl]-4-yl-3,3′-9H,9′H-bicarbazole, N,N-bis(9,9-dimethyl-9H-fluoren-2-yl)-9,9′-spirobi-9H-fluoren-1-amine, 9-[3-(triphenylsilyl)phenyl]-3,9′-bi-9H-carbazole (abbreviation: PSiCzCz), and 9′-[3-(triphenylsilyl)phenyl]-9′H-9,3′:6′,9″-tercarbazole (abbreviation: PSiCzGI); compounds having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton, such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable, have high hole-transport properties, and contribute to a reduction in driving voltage. In addition, the organic compounds given as examples of the material having a hole-transport property that can be used for the hole-transport layer can also be used.

The material having an electron-transport property preferably has an electron mobility higher than or equal to 1×10⁻⁷ cm²/Vs, further preferably higher than or equal to 1×10⁻⁶ cm²/Vs in the case where the square root of the electric field strength [V/cm] is 600. Note that any other substance can also be used as long as the substance has an electron-transport property higher than a hole-transport property.

As the material having an electron-transport property, for example, a metal complex such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), or bis[2-(2-benzothiazolyl)phenolato]zinc(II) (abbreviation: ZnBTZ); or an organic compound having a π-electron deficient heteroaromatic ring is preferably used. Examples of the organic compound having a π-electron deficient heteroaromatic ring skeleton include an organic compound that includes a heteroaromatic ring having an azole skeleton, an organic compound that includes a heteroaromatic ring having a pyridine skeleton, an organic compound that includes a heteroaromatic ring having a diazine skeleton, and an organic compound that includes a heteroaromatic ring having a triazine skeleton.

Among the above materials, the organic compound including a heteroaromatic ring having a diazine skeleton (a pyrimidine skeleton, a pyrazine skeleton, or a pyridazine skeleton), the organic compound including a heteroaromatic ring having a pyridine skeleton, and the organic compound including a heteroaromatic ring having a triazine skeleton are preferable because of their high reliability. In particular, the organic compound including a heteroaromatic ring having a diazine (pyrimidine or pyrazine) skeleton and the organic compound including a heteroaromatic ring having a triazine skeleton have a high electron-transport property and contribute to a reduction in driving voltage. A benzofuropyrimidine skeleton, a benzothienopyrimidine skeleton, a benzofuropyrazine skeleton, and a benzothienopyrazine skeleton are preferable because of their high acceptor properties and high reliability.

Preferable examples of the organic compound having a it-electron deficient heteroaromatic ring skeleton include the following organic compounds: organic compounds having an azole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOS); organic compounds that have a heteroaromatic ring having a pyridine skeleton, such as 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), bathophenanthroline (abbreviation: BPhen), bathocuproine (abbreviation: BCP), 2,9-di(naphthalen-2-yl)-4,7-diphenyl-1,10-phenanthroline (abbreviation: NBPhen), 2,2′-(1,3-phenylene)bis(9-phenyl-1,10-phenanthroline) (abbreviation: mPPhen2P), 2-[3-(2-triphenylenyl)phenyl]-1,10-phenanthroline (abbreviation: mTpPPhen), 2-phenyl-9-(2-triphenylenyl)-1,10-phenanthroline (abbreviation: Ph-TpPhen), 2-[4-(9-phenanthryl)-1-naphthyl]-1,10-phenanthroline (abbreviation: PnNPhen), and 2-[4-(2-triphenylenyl)phenyl]-1,10-phenanthroline (abbreviation: pTpPPhen); organic compounds having a diazine skeleton, such as 2-{3-[3-(N-phenyl-9H-carbazol-3-yl)-9H-carbazol-9-yl]phenyl}dibenzo[f,h]quinoxaline (abbreviation: 2mPCCzPDBq), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 9-[3′-(dibenzothiophen-4-yl)biphenyl-3-yl]naphtho[1′,2′:4,5]furo[2,3-b]pyrazine (abbreviation: 9mDBtBPNfpr), 9-[3′-(dibenzothiophen-4-yl)biphenyl-4-yl]naphtho[1′,2′:4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 8-(biphenyl-4-yl)-4-[3-(dibenzothiophen-4-yl)phenyl]-[1]benzofuro[3,2-d]pyrimidine (abbreviation: 8BP-4mDBtPBfpm), 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)(biphenyl-3-yl)]naphtho[1′,2′:4,5]furo[3,2-d]pyrimidine (abbreviation: 8mDBtBPNfpm), 8-([2,2′-binaphthalen]-6-yl)-4-[3-(dibenzothiophen-4-yl)phenyl]-[1]benzofuro[3,2-d]pyrimidine (abbreviation: 8(βN2)-4mDBtPBfpm), 2,2′-(pyridine-2,6-diyl)bis(4-phenylbenzo[h]quinazoline) (abbreviation: 2,6(P-Bqn)2Py), 2,2′-(pyridine-2,6-diyl)bis{4-[4-(2-naphthyl)phenyl]-6-phenylpyrimidine}(abbreviation: 2,6(NP-PPm)2Py), 6-(biphenyl-3-yl)-4-[3,5-bis(9H-carbazol-9-yl)phenyl]-2-phenylpyrimidine (abbreviation: 6mBP-4Cz2PPm), 2,6-bis(4-naphthalen-1-ylphenyl)-4-[4-(3-pyridyl)phenyl]pyrimidine (abbreviation: 2,4NP-6PyPPm), 4-[3,5-bis(9H-carbazol-9-yl)phenyl]-2-phenyl-6-(biphenyl-4-yl)pyrimidine (abbreviation: 6BP-4Cz2PPm), 7-[4-(9-phenyl-9H-carbazol-2-yl)quinazolin-2-yl]-7H-dibenzo[c,g]carbazole (abbreviation: PC-cgDBCzQz), 8-(p-terphenyl-3-yl)-4-[3-(dibenzothiophen-4-yl)phenyl]-[1]benzofuro[3,2-d]pyrimidine (abbreviation: 8mpTP-4mDBtPBfpm), and 11-[3′-(dibenzothiophen-4-yl)biphenyl-3-yl]phenanthro[9′,10′:4,5]furo[2,3-b]pyrazine (abbreviation: 1lmDBtBPPnfpr); and organic compounds including a heteroaromatic ring having a triazine skeleton, such as 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 2-[3′-(9,9-dimethyl-9H-fluoren-2-yl)biphenyl-3-yl]-4,6-diphenyl-1,3,5-triazine (abbreviation: mFBPTzn), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris[3′-(pyridin-3-yl)biphenyl-3-yl]-1,3,5-triazine (abbreviation: TmPPPyTz), 2-[3-(2,6-dimethyl-3-pyridinyl)-5-(9-phenanthryl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-[8-([1,1′:4′,1″-terphenyl]-4-yl)-1-dibenzofuranyl]-1,3,5-triazine (abbreviation: mBP-TPDBfTzn), 2-[4-(2-naphthyl)phenyl]-4-phenyl-6-spiro[9H-fluorene-9,9′-[9H]xanthen]-4-yl-1,3,5-triazine (abbreviation: PNP-SFx(4)Tzn), 9,9′-{6-[3-(triphenylsilyl)phenyl]-1,3,5-triazine-2,4-diyl}bis(9H-carbazole) (abbreviation: SiTrzCz2), 2-phenyl-4,6-bis[3-(triphenylsilyl)phenyl]-1,3,5-triazine (abbreviation: mSiTrz), 11-[4-(biphenyl-4-yl)-6-phenyl-1,3,5-triazin-2-yl]-11,12-dihydro-12-(biphenyl-3-yl)indolo[2,3-a]carbazole (abbreviation: BP-mBPIcz(II)Tzn), 3-{3-[9-(4,6-diphenyl-1,3,5-triazin-2-yl)-2-dibenzofuranyl]phenyl}-9-phenyl-9H-carbazole (abbreviation: mPCPDBfTzn), 9,9′-[6-(biphenyl-4-yl)-2-phenyl-1,3,5-triazine-4,3″-diyl]bis(9H-carbazole) (abbreviation: Cz-pmCzBPTzn), 3-phenyl-9-[4-phenyl-6-(9-phenyl-3-dibenzofuranyl)-1,3,5-triazin-2-yl]-9H-carbazole (abbreviation: PDBf-PCzTzn), and 9-[4-(4,6-diphenyl-1,3,5-triazin-2-yl)-2-dibenzothienyl]-2-phenyl-9H-carbazole (abbreviation: PCzDBtTzn). The organic compound including a heteroaromatic ring having a diazine skeleton, the organic compound including a heteroaromatic ring having a pyridine skeleton, and the organic compound including a heteroaromatic ring having a triazine skeleton are preferable because of their high reliability. In particular, the organic compound including a heteroaromatic ring having a diazine (pyrimidine or pyrazine) skeleton and the organic compound including a heteroaromatic ring having a triazine skeleton have a high electron-transport property and contribute to a reduction in driving voltage.

Note that the organic compound represented by General Formula (G1) or (G2) disclosed in Embodiment 1 can also be suitably used as a host material having an electron-transport property.

As the TADF material that can be used as the host material, any of the above materials mentioned as the TADF material can be similarly used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S₁ level of the TADF material is preferably higher than that of the fluorescent substance in order that high emission efficiency can be achieved. Furthermore, the T₁ level of the TADF material is preferably higher than the S₁ level of the fluorescent substance. Thus, the T₁ level of the TADF material is preferably higher than that of the fluorescent substance.

It is also preferable to use a TADF material that emits light whose wavelength overlaps with the wavelength of the lowest-energy absorption band of the fluorescent substance. This enables smooth transfer of excitation energy from the TADF material to the fluorescent substance and accordingly enables efficient light emission, which is preferable.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. To that end, the fluorescent substance preferably has a protective group around a luminophore (a skeleton which causes light emission) of the fluorescent substance. As the protective group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be made away from each other with little influence on carrier transport or carrier recombination. Here, the luminophore refers to an atomic group (skeleton) that brings about light emission in a fluorescent substance. The luminophore is preferably a skeleton having a π bond, further preferably has an aromatic ring, and still further preferably has a condensed aromatic ring or a condensed heteroaromatic ring. Examples of such a luminophore include a phenanthrene skeleton, a stilbene skeleton, an acridone skeleton, a phenoxazine skeleton, a phenothiazine skeleton, a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferable because of its high fluorescence quantum yield.

In the case where a fluorescent substance is used as the light-emitting substance, a substance having an acene skeleton, especially an anthracene skeleton is suitably used as the host material. The use of a substance having an anthracene skeleton as the host material for the fluorescent substance enables a light-emitting layer with high emission efficiency and high durability. Among the substances having an anthracene skeleton that is used as the host material, a substance having a diphenylanthracene skeleton, in particular, a substance having a 9,10-diphenylanthracene skeleton, is chemically stable and thus is preferably used as the host material. The host material preferably has a carbazole skeleton because the hole-injection and hole-transport properties are improved; further preferably, the host material has a benzocarbazole skeleton in which a benzene ring is condensed to a carbazole skeleton because the HOMO level thereof is higher than that of the host material having a carbazole skeleton by approximately 0.1 eV and thus holes enter the host material easily. In particular, the host material preferably has a dibenzocarbazole skeleton because the HOMO level thereof is higher than that of the host material having a carbazole skeleton by approximately 0.1 eV so that holes enter the host material easily, the hole-transport property is improved, and the heat resistance is increased. Accordingly, a substance having both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of the carbazole skeleton, a benzofluorene skeleton or a dibenzofluorene skeleton may be used. Furthermore, a dibenzofuran skeleton is preferably included, in which case the reliability can be ensured without a reduction in the T₁ level.

Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthryl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4-(9-phenyl-9H-fluoren-9-yl)biphenyl-4′-yl]anthracene (abbreviation: FLPPA), 9-(1-naphthyl)-10-[4-(2-naphthyl)phenyl]anthracene (abbreviation: αN-βNPAnth), 9-(1-naphthyl)-10-(2-naphthyl)anthracene (abbreviation: α,βADN), 2-(10-phenylanthracen-9-yl)dibenzofuran, 2-(10-phenyl-9-anthryl)benzo[b]naphtho[2,3-d]furan (abbreviation: Bnf(II)PhA), 9-(2-naphthyl)-10-[3-(2-naphthyl)phenyl]anthracene (abbreviation: βN-mβNPAnth), and 1-{4-[10-(biphenyl-4-yl)-9-anthryl]phenyl}-2-ethyl-1H-benzimidazole (abbreviation: EtBImPBPhA). In particular, CzPA, cgDBCzPA, 2mBnfPPA, and PCzPA exhibit excellent properties and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property is preferably 1:19 to 19:1.

Note that a phosphorescent substance can be used as part of the mixed material. When a fluorescent substance is used as the light-emitting substance, the phosphorescent substance can be used as an energy donor for supplying excitation energy to the fluorescent substance.

An exciplex may be formed of these mixed materials. These mixed materials are preferably selected so as to form an exciplex that exhibits light emission whose wavelength overlaps with the wavelength of a lowest-energy-side absorption band of the light-emitting substance, in which case energy can be transferred smoothly and light emission can be obtained efficiently. The use of such a structure is preferable because the driving voltage can also be reduced.

In the case where at least one of the materials forming an exciplex is a phosphorescent substance, triplet excitation energy can be efficiently converted into singlet excitation energy by reverse intersystem crossing.

Combination of a material having an electron-transport property and a material having a hole-transport property whose HOMO level is higher than or equal to that of the material having an electron-transport property is preferable for forming an exciplex efficiently. In addition, the LUMO level of the material having a hole-transport property is preferably higher than or equal to that of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed, for example, in the following manners: when the emission spectrum of the material having a hole-transport property, the emission spectrum of the material having an electron-transport property, and the emission spectrum of a mixed film of these materials are compared, it is observed that the emission spectrum of the mixed film is shifted to the longer wavelength than the emission spectrum of each of the material having a hole-transport property and the material having an electron-transport property (or has another peak on the longer wavelength side). Alternatively, when the transient photoluminescence (PL) of the material having a hole-transport property, the transient PL of the material having an electron-transport property, and the transient PL of the mixed film of these materials are compared, a difference in transient response is observed, for example, the transient PL lifetime of the mixed film has a longer lifetime component or has a larger portion of a delayed component than that of each of the material having a hole-transport property and the material having an electron-transport property. The transient PL can be rephrased as transient electroluminescence (EL). That is, the formation of an exciplex can also be confirmed by comparing the transient EL of the material having a hole-transport property, the transient EL of the material having an electron-transport property, and the transient EL of the mixed film of these materials and observing a difference in transient response.

The electron-transport layer 114 includes a substance having an electron-transport property. The substance having an electron-transport property preferably has an electron mobility higher than or equal to 1×10⁻⁷ cm²/Vs, further preferably higher than or equal to 1×10⁻⁶ cm²/Vs in the case where the square root of the electric field strength [V/cm] is 600. Note that any other substance can also be used as long as the substance has an electron-transport property higher than a hole-transport property. An organic compound including a π-electron deficient heteroaromatic ring is preferable as the above organic compound. The organic compound including a π-electron deficient heteroaromatic ring is preferably one or more of an organic compound including a heteroaromatic ring having an azole skeleton, an organic compound including a heteroaromatic ring having a pyridine skeleton, an organic compound including a heteroaromatic ring having a diazine skeleton, and an organic compound including a heteroaromatic ring having a triazine skeleton.

As the substance having an electron-transport property that can be used for the electron-transport layer 114, any of the above-listed organic compounds having an electron-transport property that are each preferably used as the host material of the light-emitting layer 113 can be similarly used.

Among the above-listed organic compounds having an electron-transport property that are each preferably used as the host material, an organic compound including a heteroaromatic ring having a diazine skeleton, an organic compound including a heteroaromatic ring having a pyridine skeleton, or an organic compound including a heteroaromatic ring having a triazine skeleton is preferable because of its high reliability. In particular, the organic compound including a heteroaromatic ring having a diazine (pyrimidine or pyrazine) skeleton and the organic compound including a heteroaromatic ring having a triazine skeleton have a high electron-transport property and contribute to a reduction in driving voltage. In particular, an organic compound having a phenanthroline skeleton such as mTpPPhen, PnNPhen, or mPPhen2P is preferable, and an organic compound having a phenanthroline dimer structure such as mPPhen2P is further preferable because of its high stability.

Note that the organic compound represented by General Formula (G1) or (G2) disclosed in Embodiment 1 can also be suitably used for the electron-transport layer 114. The use of the organic compound represented by General Formula (G1) or (G2) disclosed in Embodiment 1 for the electron-transport layer 114 facilitates electron injection from the electron-injection layer 115. The organic compound represented by General Formula (G1) or (G2) disclosed in Embodiment 1 has a high electron-transport property to contribute to a reduction in driving voltage.

Note that the electron-transport layer 114 may have a stacked-layer structure. A layer in the stacked-layer structure of the electron-transport layer 114, which is in contact with the light-emitting layer 113, may function as a hole-blocking layer. In the case where the electron-transport layer in contact with the light-emitting layer functions as a hole-blocking layer, the electron-transport layer is preferably formed using a material having a lower HOMO level than a material included in the light-emitting layer 113 by 0.5 eV or more.

A layer that includes an alkali metal, an alkaline earth metal, a compound or a complex of an alkali metal or an alkaline earth metal, 1,1′-pyridine-2,6-diyl-bis(1,3,4,6,7,8-hexahydro-2H-pyrimido[1,2-a]pyrimidine) (abbreviation: hpp2Py), or the like may be provided as the electron-injection layer 115. The electron-injection layer 115 may be a layer including a substance having an electron-transport property and any of the above substances.

The electron-injection layer 115 preferably includes the organic compound represented by General Formula (G1) or (G2) disclosed in Embodiment 1. With the organic compound represented by General Formula (G1) or (G2) disclosed in Embodiment 1, electron injection can be facilitated, enabling a light-emitting device to have a low driving voltage. Furthermore, when coordinating to a metal or a metal compound, the organic compound which is represented by General Formula (G1) or (G2) disclosed in Embodiment 1 and is included in the electron-injection layer 115 can improve the electron-donor property of the metal or the metal compound. This can inhibit impairment of the function of the electron-injection layer 115 even when the electron-injection layer 115 is exposed to an air atmosphere, whereby an increase in driving voltage can be inhibited and a light-emitting device with favorable characteristics can be provided. Since the electron-injection layer including the organic compound represented by General Formula (G1) or (G2) has high heat resistance, the light-emitting device can have high reliability, particularly high heat resistance.

That is, the light-emitting device including the electron-injection layer 115 that includes the metal or the metal compound and the organic compound represented by General Formula (G1) or (G2) disclosed in Embodiment 1 can have favorable characteristics without a significant increase in driving voltage even when subjected to processing by a photolithography method involving an air exposure step.

Instead of the electron-injection layer 115, the charge-generation layer 116 can be provided (FIG. 1B). The charge-generation layer 116 refers to a layer capable of injecting holes into a layer in contact with the cathode side of the charge-generation layer 116 and electrons into a layer in contact with the anode side thereof when a potential is applied. The charge-generation layer 116 includes at least a p-type layer 117. The p-type layer 117 is preferably formed using any of the composite materials given above as examples of materials that can be used for the hole-injection layer 111. The p-type layer 117 may be formed by stacking a film including the above-described acceptor material as a material included in the composite material and a film including a hole-transport material. When a potential is applied to the p-type layer 117, electrons are injected into the electron-transport layer 114 and holes are injected into the cathode; thus, the light-emitting device is driven. Since the organic compound of one embodiment of the present invention has a low refractive index, using the organic compound for the p-type layer 117 enables the light-emitting device to have high external quantum efficiency.

Note that the charge-generation layer 116 preferably includes one or both of an electron-relay layer 118 and an electron-injection buffer layer 119 in addition to the p-type layer 117.

The electron-relay layer 118 includes at least a substance having an electron-transport property and has a function of preventing an interaction between the electron-injection buffer layer 119 and the p-type layer 117 and smoothly transferring electrons. The LUMO level of the substance having an electron-transport property included in the electron-relay layer 118 is preferably positioned between the LUMO level of the acceptor substance in the p-type layer 117 and the LUMO level of a substance included in a layer of the electron-transport layer 114 that is in contact with the charge-generation layer 116. As a specific value of the energy level, the LUMO level of the substance having an electron-transport property in the electron-relay layer 118 is preferably higher than or equal to −5.0 eV, further preferably higher than or equal to −5.0 eV and lower than or equal to −3.0 eV, still further preferably higher than or equal to −4.30 eV and lower than or equal to −3.00 eV, yet still further preferably higher than or equal to −4.30 eV and lower than or equal to −3.30 eV, in which case an increase in driving voltage can be suppressed. Note that as the substance having an electron-transport property in the electron-relay layer 118, a phthalocyanine-based material or a metal complex having a metal-oxygen bond and an aromatic ligand is preferably used.

Specific examples of the substance having an electron-transport property in the electron-relay layer 118 include a perylenetetracarboxylic acid derivative such as diquinoxalino[2,3-a:2′,3′-c]phenazine (abbreviation: HATNA), 2,3,8,9,14,15-hexafluorodiquinoxalino[2,3-a:2′,3′-c]phenazine (abbreviation: HATNA-F6), 3,4,9,10-perylenetetracarboxylic diimide (abbreviation: PTCDI), or 3,4,9,10-perylenetetracarboxyl-bis-benzimidazole (abbreviation: PTCBI), (C₆₀—Iₕ)[5,6]fullerene (abbreviation: C₆₀), and (C₇₀-D₅ₕ)[5,6]fullerene (abbreviation: C₇₀). It is also possible to use a compound including a heterophane skeleton, which is a cyclophane skeleton having a hetero ring; for example, a phthalocyanine compound such as phthalocyanine (abbreviation: H₂Pc) can be used as the compound. Moreover, it is possible to use a metal phthalocyanine including copper, zinc, cobalt, iron, chromium, nickel, or the like or a derivative thereof, such as copper phthalocyanine (abbreviation: CuPc), zinc phthalocyanine (abbreviation: ZnPc), cobalt phthalocyanine (abbreviation: CoPc), iron phthalocyanine (abbreviation: FePc), tin phthalocyanine (abbreviation: SnPc), tin oxide phthalocyanine (abbreviation: SnOPc), titanium oxide phthalocyanine (abbreviation: TiOPc), or vanadium oxide phthalocyanine (abbreviation: VOPc). It is particularly preferable to use a phthalocyanine-based metal complex such as copper phthalocyanine or zinc phthalocyanine or 2,3,8,9,14,15-hexafluorodiquinoxalino[2,3-a:2′,3′-c]phenazine.

The electron-injection buffer layer 119 can be formed using a substance having a high electron-injection property, e.g., an alkali metal, an alkaline earth metal, a rare earth metal, or a compound thereof (an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)).

In the case where the electron-injection buffer layer 119 includes a substance having an electron-transport property and an electron-donor substance, the electron-donor substance can be an organic compound such as tetrathianaphthacene (abbreviation: TTN), nickelocene, or decamethylnickelocene, as well as an alkali metal, an alkaline earth metal, a rare earth metal, or a compound thereof (e.g., an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)). As the substance having an electron-transport property, a material similar to the above-described material for the electron-transport layer 114 can be used.

The electron-injection buffer layer 119 preferably includes the organic compound represented by General Formula (G1) or (G2) disclosed in Embodiment 1. With the organic compound represented by General Formula (G1) or (G2) disclosed in Embodiment 1, electron injection can be facilitated, enabling a light-emitting device to have a low driving voltage.

Furthermore, when coordinating to a metal or a metal compound, the organic compound which is represented by General Formula (G1) or (G2) disclosed in Embodiment 1 and is included in the electron-injection buffer layer 119 can improve the electron-donor property of the metal or the metal compound. This can inhibit impairment of the function of the electron-injection buffer layer 119 even when the charge-generation layer 116 is exposed to an air atmosphere, whereby an increase in driving voltage can be inhibited and a light-emitting device with favorable characteristics can be provided.

That is, the light-emitting device provided with the charge-generation layer 116 including the electron-injection buffer layer 119 that includes the metal or the metal compound and the organic compound represented by General Formula (G1) or (G2) disclosed in Embodiment 1 can have favorable characteristics without a significant increase in driving voltage even when subjected to processing by a photolithography method involving an air exposure step.

Since the electron-injection buffer layer 119 including the organic compound represented by General Formula (G1) or (G2) has high heat resistance, the light-emitting device can have high reliability, particularly high heat resistance. Since processing by a photolithography method often involves a heating step to remove moisture, the light-emitting device using the electron-injection buffer layer 119 including the organic compound represented by General Formula (G1) or (G2) can be further suitable for the processing by a photolithography method.

The second electrode 102 is an electrode including a cathode. The second electrode 102 may have a stacked-layer structure, in which case a layer in contact with the organic compound layer 103 functions as a cathode. For the cathode, a metal, an alloy, an electrically conductive compound, or a mixture thereof each having a low work function (specifically, lower than or equal to 3.8 eV) can be used, for example. Specific examples of such a cathode material include elements belonging to Groups 1 and 2 of the periodic table, such as alkali metals (e.g., lithium (Li) and cesium (Cs)), magnesium (Mg), calcium (Ca), and strontium (Sr), alloys including these elements (e.g., MgAg and AlLi), compounds including these elements (e.g., lithium fluoride (LiF), cesium fluoride (CsF), and calcium fluoride (CaF₂)), rare earth metals such as europium (Eu) and ytterbium (Yb), and alloys including these rare earth metals. However, when the electron-injection layer 115 or a thin film formed using any of the above materials having a low work function is provided between the second electrode 102 and the electron-transport layer, a variety of conductive materials such as Al, Ag, ITO, and indium oxide-tin oxide including silicon or silicon oxide can be used for the cathode regardless of the work function.

When the second electrode 102 is formed using a material that transmits visible light, the light-emitting device can emit light from the second electrode 102 side. Furthermore, light extraction efficiency can be improved by forming, over the second electrode 102, a cap layer using a material with a high refractive index (e.g., an ordinary refractive index (nₒ) at a wavelength of 450 nm is greater than or equal to 1.90, an ordinary refractive index (nₒ) at a wavelength of 520 nm is greater than or equal to 1.80, or an ordinary refractive index (nₒ) at a wavelength of 630 nm is greater than or equal to 1.75). Note that an organic compound is preferably used for the cap layer, in which case the cap layer is easily formed.

Films of these conductive materials can be formed by a dry process such as a vacuum evaporation method or a sputtering method, an ink-jet method, a spin coating method, or the like. Alternatively, a wet process using a sol-gel method or a wet process using a paste of a metal material may be employed.

The organic compound layer 103 can be formed by any of a variety of methods, including a dry process and a wet process. For example, a vacuum evaporation method, a gravure printing method, an offset printing method, a screen printing method, an ink-jet method, a spin coating method, or the like may be used.

Different film formation methods may be used to form the electrodes or the layers described above.

Next, an embodiment of a light-emitting device with a structure in which a plurality of light-emitting units are stacked (this type of light-emitting device is also referred to as a stacked device or a tandem device) is described with reference to FIG. 1C. This light-emitting device includes a plurality of light-emitting units between an anode and a cathode. One light-emitting unit has substantially the same structure as the organic compound layer 103 shown in FIG. 1A. In other words, the light-emitting device illustrated in FIG. 1C includes a plurality of light-emitting units, and the light-emitting device illustrated in FIG. 1A or 1B includes a single light-emitting unit.

In FIG. 1C, a first light-emitting unit 511 and a second light-emitting unit 512 are stacked between a first electrode 501 and a second electrode 502, and an intermediate layer 513 is provided between the first light-emitting unit 511 and the second light-emitting unit 512. The first electrode 501 and the second electrode 502 respectively correspond to the first electrode 101 and the second electrode 102 illustrated in FIG. 1A, and the description made for FIG. 1A can be similarly applied thereto. Furthermore, the first light-emitting unit 511 and the second light-emitting unit 512 may be formed using the same material or different materials.

The intermediate layer 513 has a function of injecting electrons into one of the light-emitting units and injecting holes into the other of the light-emitting units when voltage is applied between the first electrode 501 and the second electrode 502. That is, in FIG. 1C, the intermediate layer 513 can inject electrons into the first light-emitting unit 511 and holes into the second light-emitting unit 512 when voltage is applied such that the potential of the anode becomes higher than the potential of the cathode.

The intermediate layer 513 preferably has a structure similar to that of the charge-generation layer 116 described with reference to FIG. 1B. A composite material of an organic compound and a metal oxide enables low-voltage driving and low-current driving because of having an excellent carrier-injection property and an excellent carrier-transport property.

In particular, the electron-injection buffer layer 119 of the intermediate layer 513 preferably includes the organic compound represented by General Formula (G1) or (G2) disclosed in Embodiment 1. With the organic compound represented by General Formula (G1) or (G2) disclosed in Embodiment 1, electron injection can be facilitated, enabling a light-emitting device to have a low driving voltage.

Furthermore, when coordinating to a metal or a metal compound, the organic compound which is represented by General Formula (G1) or (G2) disclosed in Embodiment 1 and is included in the electron-injection buffer layer 119 can improve the electron-donor property of the metal or the metal compound. This can inhibit impairment of the function of the electron-injection buffer layer 119 of the intermediate layer 513 even when the organic compound layer 103 is exposed to an air atmosphere, whereby an increase in driving voltage can be inhibited and a light-emitting device with favorable characteristics can be provided.

That is, the tandem light-emitting device provided with the intermediate layer 513 including the electron-injection buffer layer 119 that includes the metal or the metal compound and the organic compound represented by General Formula (G1) or (G2) disclosed in Embodiment 1 can have favorable characteristics without a significant increase in driving voltage even when subjected to processing by a photolithography method involving an air exposure step.

Since the electron-injection buffer layer 119 including the organic compound represented by General Formula (G1) or (G2) has high heat resistance, the light-emitting device can have high reliability, particularly high heat resistance. Since processing by a photolithography method often involves a heating step to remove moisture, the light-emitting device using the electron-injection buffer layer 119 including the organic compound represented by General Formula (G1) or (G2) can be further suitable for the processing by a photolithography method.

In the case where the anode-side surface of a light-emitting unit is in contact with the intermediate layer 513, the intermediate layer 513 can also function as a hole-injection layer of the light-emitting unit; thus, a hole-injection layer may not be necessarily provided in the light-emitting unit.

In the case where the electron-injection buffer layer 119 is provided in the intermediate layer 513, the electron-injection buffer layer 119 functions as the electron-injection layer in the light-emitting unit on the anode side; thus, an electron-injection layer may not be necessarily formed in the light-emitting unit on the anode side.

The light-emitting device having two light-emitting units is described with reference to FIG. 1C; however, one embodiment of the present invention can also be applied to a light-emitting device in which three or more light-emitting units are stacked. With a plurality of light-emitting units partitioned by the intermediate layer 513 between a pair of electrodes as in the light-emitting device of this embodiment, it is possible to provide a long-life element that can emit light with high luminance at a low current density. Moreover, a light-emitting apparatus that can be driven at a low voltage and has low power consumption can also be provided.

When the emission colors of the light-emitting units are different, light emission of a desired hue can be obtained from the light-emitting device as a whole. For example, in a light-emitting device having two light-emitting units, the emission colors of the first light-emitting unit may be red and green and the emission color of the second light-emitting unit may be blue, so that the light-emitting device can emit white light as a whole. When the emission center substances included in the light-emitting units exhibit the same emission color, a light-emitting device with extremely high current efficiency can be provided.

The organic compound layer 103, the first light-emitting unit 511, the second light-emitting unit 512, the layers such as the charge-generation layer, and the electrodes described above can be formed by a method such as an evaporation method (including a vacuum evaporation method), a droplet discharge method (also referred to as an ink-jet method), a coating method, or a gravure printing method. A low molecular material, a middle molecular material (including an oligomer and a dendrimer), or a high molecular material may be included in the above components.

### Embodiment 3

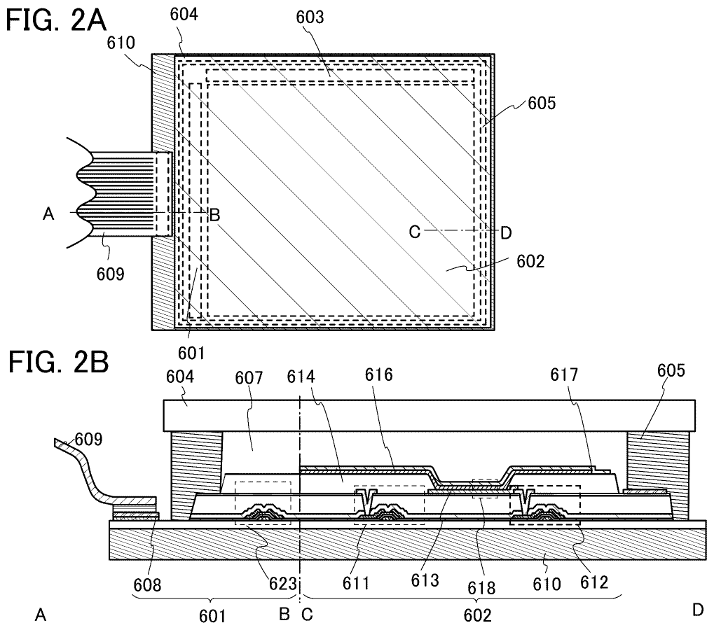

In this embodiment, the display device manufactured using the light-emitting device described in Embodiment 2 is described with reference to FIGS. 2A and 2B. Note that FIG. 2A is a top view of the display device and FIG. 2B is a cross-sectional view taken along the lines A-B and C-D in FIG. 2A. This display device includes a driver circuit portion (source line driver circuit) 601, a pixel portion 602, and a driver circuit portion (gate line driver circuit) 603 to control light emission of a light-emitting device, shown with dotted lines. Reference numeral 604 denotes a sealing substrate, reference numeral 605 denotes a sealing material; and reference numeral 607 denotes a space surrounded by the sealing material 605.

A lead wiring 608 denotes a wiring for transmitting signals to be input to the source line driver circuit 601 and the gate line driver circuit 603 and receiving signals such as a video signal, a clock signal, a start signal, and a reset signal from a flexible printed circuit (FPC) 609 serving as an external input terminal. Although only the FPC is illustrated here, a printed wiring board (PWB) may be attached to the FPC. The display device in this specification includes, in its category, not only the display device itself but also the display device provided with the FPC or the PWB.

Next, a cross-sectional structure is described with reference to FIG. 2B. The driver circuit portions and the pixel portion are formed over an element substrate 610; FIG. 2B illustrates the source line driver circuit 601, which is a driver circuit portion, and one pixel in the pixel portion 602.

The element substrate 610 may be a substrate formed of glass, quartz, an organic resin, a metal, an alloy, or a semiconductor or a plastic substrate formed of fiber reinforced plastic (FRP), polyvinyl fluoride (PVF), polyester, or an acrylic resin.

The structure of transistors used in the pixels and the driver circuits is not particularly limited. For example, inverted staggered transistors may be used, or staggered transistors may be used. In addition, top-gate transistors or bottom-gate transistors may be used. A semiconductor material used for the transistors is not particularly limited, and for example, silicon, germanium, silicon carbide, gallium nitride, or the like can be used. Alternatively, an oxide semiconductor including at least one of indium, gallium, and zinc, such as an In—Ga—Zn-based metal oxide, may be used.

There is no particular limitation on the crystallinity of a semiconductor material used for the transistors, and an amorphous semiconductor or a semiconductor having crystallinity (a microcrystalline semiconductor, a polycrystalline semiconductor, a single crystal semiconductor, or a semiconductor partly including crystal regions) can be used. It is preferable to use a semiconductor having crystallinity, in which case degradation of transistor characteristics can be inhibited.

Here, an oxide semiconductor is preferably used for semiconductor devices such as the transistors provided in the pixels and the driver circuits and transistors used for touch sensors described later, and the like. In particular, an oxide semiconductor having a wider band gap than silicon is preferably used. When an oxide semiconductor having a wider band gap than silicon is used, the off-state current of the transistors can be reduced.

The oxide semiconductor preferably includes at least indium (In) or zinc (Zn). Further preferably, the oxide semiconductor includes an oxide represented by an In-M-Zn-based oxide (M represents a metal such as Al, Ti, Ga, Ge, Y, Zr, Sn, La, Ce, or Hf).

As a semiconductor layer, it is particularly preferable to use an oxide semiconductor film including a plurality of crystal parts whose c-axes are aligned perpendicular to a surface on which the semiconductor layer is formed or the top surface of the semiconductor layer and in which the adjacent crystal parts have no grain boundary.

The use of such a material for the semiconductor layer makes it possible to provide a highly reliable transistor in which a change in the electrical characteristics is suppressed.

Charge accumulated in a capacitor through a transistor including the above-described semiconductor layer can be held for a long time because of the low off-state current of the transistor. When such a transistor is used in a pixel, operation of a driver circuit can be stopped while a gray scale of an image in each display region is maintained. As a result, an electronic appliance with extremely low power consumption can be obtained.

For stable characteristics of the transistor and the like, a base film is preferably provided. The base film can be formed with a single-layer structure or a stacked-layer structure using an inorganic insulating film such as a silicon oxide film, a silicon nitride film, a silicon oxynitride film, or a silicon nitride oxide film. The base film can be formed by a sputtering method, a chemical vapor deposition (CVD) method (e.g., a plasma CVD method, a thermal CVD method, or a metal organic CVD (MOCVD) method), an atomic layer deposition (ALD) method, a coating method, a printing method, or the like. Note that the base film is not necessarily provided.

Note that an FET 623 is shown as a transistor formed in the driver circuit portion 601. In addition, the driver circuit may be formed with any of a variety of circuits such as a CMOS circuit, a PMOS circuit, or an NMOS circuit. Although a driver integrated type in which the driver circuit is formed over the substrate is described in this embodiment, the driver circuit is not necessarily formed over the substrate, and can be formed outside.

The pixel portion 602 includes a plurality of pixels including a switching FET 611, a current controlling FET 612, and a first electrode 613 electrically connected to a drain of the current controlling FET 612. One embodiment of the present invention is not limited to the structure, and the pixel portion 602 may include three or more FETs and a capacitor in combination.

Note that an insulator 614 is formed to cover an end portion of the first electrode 613. Here, the insulator 614 can be formed using a positive photosensitive acrylic resin film.

In order to improve coverage with an organic compound layer or the like which is formed later, the insulator 614 is formed to have a curved surface with curvature at its upper or lower end portion. For example, in the case where a positive photosensitive acrylic resin is used as a material of the insulator 614, only the upper end portion of the insulator 614 preferably has a curved surface with a curvature radius (0.2 μm to 3 μm). For the insulator 614, a negative photosensitive resin or a positive photosensitive resin can be used.

An organic compound layer 616 and a second electrode 617 are formed over the first electrode 613. Here, as a material used for the first electrode 613 functioning as an anode, a material having a high work function is preferably used. For example, a single-layer film of an ITO film, an indium tin oxide film including silicon, an indium oxide film including zinc oxide at 2 wt % to 20 wt %, a titanium nitride film, a chromium film, a tungsten film, a Zn film, a Pt film, or the like, a stack of a titanium nitride film and a film including aluminum as its main component, a stack of three layers of a titanium nitride film, a film including aluminum as its main component, and a titanium nitride film, or the like can be used. The stacked-layer structure enables low wiring resistance, favorable ohmic contact, and a function as an anode.

The organic compound layer 616 is formed by any of a variety of methods such as an evaporation method using an evaporation mask, an ink-jet method, and a spin coating method. The organic compound layer 616 has the structure described in Embodiment 2. As another material included in the organic compound layer 616, a low molecular compound or a high molecular compound (including an oligomer or a dendrimer) may be used.

As a material used for the second electrode 617, which is formed over the organic compound layer 616 and functions as a cathode, a material having a low work function (e.g., Al, Mg, Li, and Ca, or an alloy or a compound thereof, such as MgAg, MgIn, and AlLi) is preferably used. In the case where light generated in the organic compound layer 616 is transmitted through the second electrode 617, a stack of a thin metal film and a transparent conductive film (e.g., ITO, indium oxide including zinc oxide at 2 wt % to 20 wt %, indium tin oxide including silicon, or zinc oxide (ZnO)) is preferably used for the second electrode 617.

Note that the light-emitting device is formed with the first electrode 613, the organic compound layer 616, and the second electrode 617. The light-emitting device is the light-emitting device described in Embodiment 2. In the display device of this embodiment, the pixel portion, which includes a plurality of light-emitting devices, may include both the light-emitting device described in Embodiment 2 and a light-emitting device having a different structure.

The sealing substrate 604 is bonded to the element substrate 610 with the sealing material 605, so that a light-emitting device 618 is provided in the space 607 surrounded by the element substrate 610, the sealing substrate 604, and the sealing material 605. The space 607 may be filled with a filler, or may be filled with an inert gas (such as nitrogen or argon), or the sealing material. The structure of the sealing substrate in which a recessed portion is formed and a desiccant is provided in the recessed portion is preferable because deterioration due to the influence of moisture can be inhibited.

An epoxy-based resin or glass frit is preferably used for the sealing material 605. It is desirable that such a material not be permeable to moisture or oxygen as much as possible. As the sealing substrate 604, a glass substrate, a quartz substrate, or a plastic substrate formed of fiber reinforced plastic (FRP), polyvinyl fluoride (PVF), polyester, and acrylic resin can be used.

Although not illustrated in FIGS. 2A and 2B, a cap layer and/or a protective film may be provided over the second electrode. Forming the cap layer can improve the light extraction efficiency. The cap layer is preferably formed using a material with an ordinary refractive index (nₒ) of higher than or equal to 1.90 at a wavelength of 450 nm, a material with an ordinary refractive index (nₒ) of higher than or equal to 1.80 at a wavelength of 520 nm, or a material with an ordinary refractive index (nₒ) of higher than or equal to 1.75 at a wavelength of 630 nm, for example. The cap layer is preferably formed by depositing an organic compound by an evaporation method, in which case the cap layer can be easily formed.

As the protective film, an organic resin film or an inorganic insulating film may be formed. In particular, a material that can be formed by an atomic layer deposition (ALD) method is preferably used for the protective film. A dense protective film having reduced defects such as cracks or pinholes or a uniform thickness can be formed by an ALD method. Furthermore, damage caused to a process member in forming the protective film can be reduced.

The protective film may be formed so as to cover an exposed portion of the sealing material 605. The protective film may be provided so as to cover surfaces and side surfaces of the pair of substrates and exposed side surfaces of a sealing layer, an insulating layer, and the like.

The protective film can be formed using a material that is less likely to transmit an impurity such as water easily. Thus, diffusion of an impurity such as water from the outside into the inside can be effectively suppressed.

As a material for the protective film, an oxide, a nitride, a fluoride, a sulfide, a ternary compound, a metal, a polymer, or the like can be used. For example, the material may include aluminum oxide, hafnium oxide, hafnium silicate, lanthanum oxide, silicon oxide, strontium titanate, tantalum oxide, titanium oxide, zinc oxide, niobium oxide, zirconium oxide, tin oxide, yttrium oxide, cerium oxide, scandium oxide, erbium oxide, vanadium oxide, indium oxide, aluminum nitride, hafnium nitride, silicon nitride, tantalum nitride, titanium nitride, niobium nitride, molybdenum nitride, zirconium nitride, gallium nitride, a nitride including titanium and aluminum, an oxide including titanium and aluminum, an oxide including aluminum and zinc, a sulfide including manganese and zinc, a sulfide including cerium and strontium, an oxide including erbium and aluminum, an oxide including yttrium and zirconium, or the like. Note that aluminum oxide is particularly preferable for the protective film.

The protective film is preferably formed using a film formation method with favorable step coverage. One such method is an ALD method. By an ALD method, a uniform protective film with few defects can be formed even on, for example, a surface with a complex uneven shape or top, side, and rear surfaces of a touch panel.

As described above, the display device manufactured using the light-emitting device described in Embodiment 2 can be obtained.

The display device in this embodiment is manufactured using the light-emitting device described in Embodiment 2 and thus can have favorable characteristics. Specifically, since the light-emitting device described in Embodiment 2 has low driving voltage, the display device can achieve low power consumption. Since the light-emitting device described in Embodiment 2 has high reliability, the display device can be highly reliable. In addition, the light-emitting device described in Embodiment 2 enables the display device to have high display quality.

This embodiment can be freely combined with any of the other embodiments.

### Embodiment 4

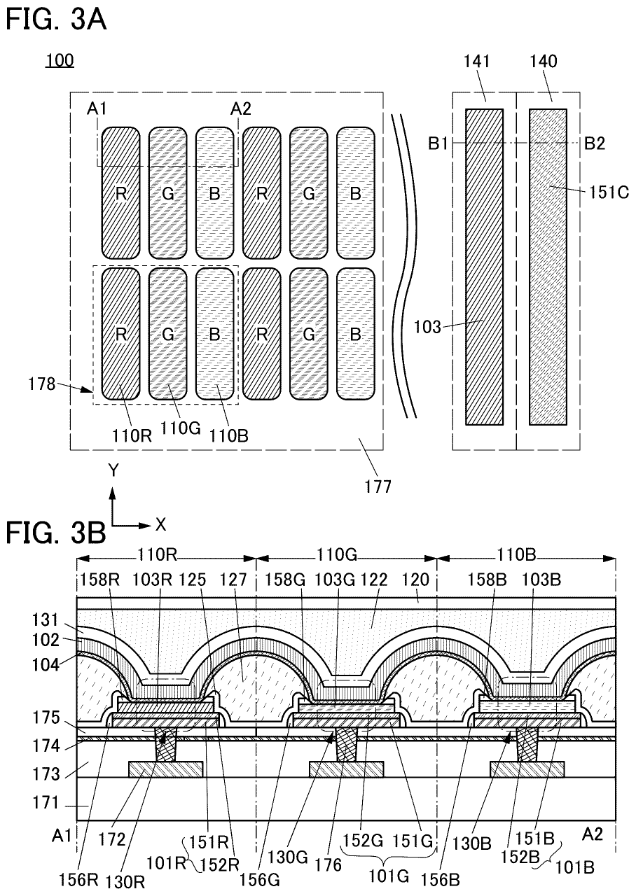

As illustrated in FIGS. 3A and 3B, a plurality of light-emitting devices 130 are formed over an insulating layer 175 to constitute a display device. In this embodiment, the display device of another embodiment of the present invention will be described in detail.

A display device 100 includes a pixel portion 177 in which a plurality of pixels 178 are arranged in a matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and Y, and four subpixels emitting light of R, G, and B and infrared light (IR).

In this specification and the like, the row direction and the column direction are sometimes referred to as the X direction and the Y direction, respectively. The X direction and the Y direction intersect with each other and are perpendicular to each other, for example.

FIG. 3A illustrates an example where subpixels of different colors are arranged in the X direction and subpixels of the same color are arranged in the Y direction. Note that subpixels of different colors may be arranged in the Y direction, and subpixels of the same color may be arranged in the X direction.

Outside the pixel portion 177, a connection portion 140 is provided and a region 141 may also be provided. In the case where the region 141 is provided, the region 141 is provided between the pixel portion 177 and the connection portion 140. In the case where the region 141 is provided, an organic compound layer is provided in the region 141. A conductive layer 151C is provided in the connection portion 140.

Although FIG. 3A illustrates an example where the region 141 and the connection portion 140 are positioned on the right side of the pixel portion 177, the positions of the region 141 and the connection portion 140 are not particularly limited. The number of the regions 141 and the number of the connection portions 140 can each be one or more.

FIG. 3B is an example of a cross-sectional view along the dashed-dotted line A1-A2 in FIG. 3A. As illustrated in FIG. 3B, the display device 100 includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 131 is provided to cover the light-emitting device 130. A substrate 120 is bonded to the protective layer 131 with a resin layer 122. An inorganic insulating layer 125 and an insulating layer 127 over the inorganic insulating layer 125 are preferably provided between the adjacent light-emitting devices 130.

Although FIG. 3B illustrates cross sections of a plurality of the inorganic insulating layers 125 and a plurality of the insulating layers 127, the inorganic insulating layers 125 are preferably connected to each other and the insulating layers 127 are preferably connected to each other when the display device 100 is seen from above.

In FIG. 3B, a light-emitting device 130R, a light-emitting device 130G, and a light-emitting device 130B are illustrated as the light-emitting devices 130. The light-emitting devices 130R, 130G, and 130B exhibit different emission colors. For example, the light-emitting device 130R can emit red light, the light-emitting device 130G can emit green light, and the light-emitting device 130B can emit blue light. Alternatively, the light-emitting device 130R, the light-emitting device 130G, or the light-emitting device 130B may emit visible light of another color or infrared light.

The display device of one embodiment of the present invention can be, for example, a top-emission display device where light is emitted in the direction opposite to a substrate over which light-emitting devices are formed. Note that the display device of one embodiment of the present invention may be of a bottom emission type.

The light-emitting device 130R includes a first electrode 101R (pixel electrode) including a conductive layer 151R and a conductive layer 152R, an organic compound layer 103R over the first electrode 101R, the common layer 104 over the organic compound layer 103R, and the second electrode 102 (common electrode) over the common layer 104. Although the common layer 104 is not necessarily provided, it is preferable to provide the common layer 104 to reduce damage to the organic compound layer 103R during processing.

The light-emitting device 130G includes a first electrode 101G (pixel electrode) including a conductive layer 151G and a conductive layer 152G, an organic compound layer 103G over the first electrode 101G, the common layer 104 over the organic compound layer 103G, and the second electrode 102 (common electrode) over the common layer 104. Although the common layer 104 is not necessarily provided, it is preferable to provide the common layer 104 to reduce damage to the organic compound layer 103G during processing.

The light-emitting device 130B has a structure described in Embodiment 2. The light-emitting device 130B includes a first electrode 101B (pixel electrode) including a conductive layer 151B and a conductive layer 152B, an organic compound layer 103B over the first electrode 101B, the common layer 104 over the organic compound layer 103B, and the second electrode 102 (common electrode) over the common layer 104. Although the common layer 104 is not necessarily provided, it is preferable to provide the common layer 104 to reduce damage to the organic compound layer 103B during processing. Furthermore, in the case where the common layer 104 is provided, a stack of the organic compound layer 103B and the common layer 104 corresponds to the organic compound layer 103 described in Embodiment 2. In the case where the common layer 104 is not provided, the first layer 135 corresponds to the organic compound layer 103.

Note that the common layer 104 is preferably an electron-injection layer or an electron-transport layer, further preferably an electron-injection layer. In the case where the common layer 104 is an electron-transport layer, it is preferable that the electron-transport layer have a stacked-layer structure, and it is further preferable that, among the stacked layers, a layer on the second electrode side be the common layer 104 and a layer on the light-emitting layer side be the organic compound layer 103.

Since the light-emitting devices 130R and 130G are manufactured through a photolithography process, such a structure can suppress an increase in driving voltage due to the photolithography process and enables the light-emitting devices to have a low driving voltage.

In the light-emitting device 130, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layers 103R, 103G, and 103B are island-shaped layers that are independent of each other on a light-emitting device basis or on an emission color basis. Providing the island-shaped organic compound layer 103 in each of the light-emitting devices 130 can inhibit leakage current between the adjacent light-emitting devices 130 even in a high-resolution display device. This can prevent crosstalk, so that a display device with extremely high contrast can be obtained. Specifically, a display device having high current efficiency at low luminance can be obtained.

The island-shaped organic compound layer 103 is formed by forming an organic compound film and processing the organic compound film by a photolithography method.

The organic compound layer 103 is preferably provided to cover the top surface and the side surface of the first electrode (pixel electrode) of the light-emitting device 130. In this case, the aperture ratio of the display device 100 can be easily increased as compared to the structure where an end portion of the organic compound layer 103 is positioned inward from an end portion of the pixel electrode. Covering the side surface of the pixel electrode of the light-emitting device 130 with the organic compound layer 103 can inhibit the pixel electrode from being in contact with the second electrode 102; hence, a short circuit of the light-emitting device 130 can be inhibited.

In the display device of one embodiment of the present invention, the first electrode (pixel electrode) of the light-emitting device preferably has a stacked-layer structure. For example, in the example illustrated in FIG. 3B, the first electrode of the light-emitting device 130 is a stack of the conductive layer 151 and the conductive layer 152.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy including an appropriate combination of any of these metals, for example.

For the conductive layer 152, an oxide including one or more selected from indium, tin, zinc, gallium, titanium, aluminum, and silicon can be used. For example, it is preferable to use a conductive oxide including one or more of indium oxide, indium tin oxide, indium zinc oxide, zinc oxide, zinc oxide including gallium, titanium oxide, indium zinc oxide including gallium, indium zinc oxide including aluminum, indium tin oxide including silicon, indium zinc oxide including silicon, and the like. In particular, an indium tin oxide including silicon can be suitably used for the conductive layer 152 because of having a work function of higher than or equal to 4.0 eV, for example.

The conductive layer 151 and the conductive layer 152 may each be a stack of a plurality of layers including different materials. In that case, the conductive layer 151 may include a layer formed using a material that can be used for the conductive layer 152, such as a conductive oxide, and the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

The conductive layer 151 preferably has a tapered side surface. Specifically, the side surface of the conductive layer 151 preferably has a tapered shape with a taper angle of less than 90°. In that case, the conductive layer 152 provided along the side surface of the conductive layer 151 also has a tapered shape. When the side surface of the conductive layer 152 has a tapered shape, coverage with the organic compound layer 103 provided along the side surface of the conductive layer 152 can be improved.

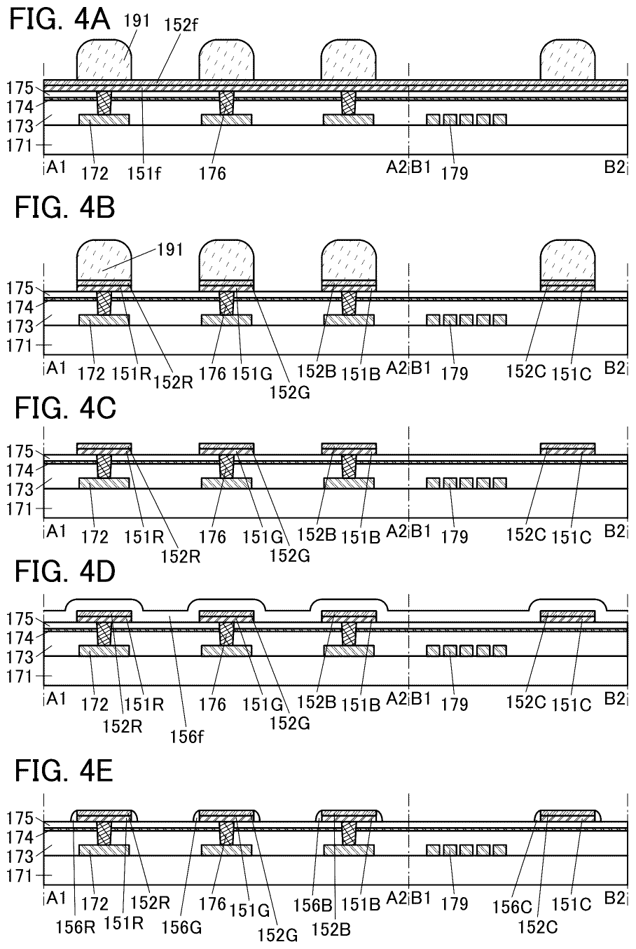

Next, an exemplary method for manufacturing the display device 100 having the structure shown in FIG. 3A is described with reference to FIGS. 4A to 4E, FIGS. 5A and 5B, FIGS. 6A to 6D, FIGS. 7A to 7C, FIGS. 8A to 8C, and FIGS. 9A to 9C.

### Manufacturing Method Example 1

Thin films included in the display device (e.g., insulating films, semiconductor films, and conductive films) can be formed by a sputtering method, a chemical vapor deposition (CVD) method, a vacuum evaporation method, a pulsed laser deposition (PLD) method, an ALD method, or the like.

Thin films included in the display device (e.g., insulating films, semiconductor films, and conductive films) can also be formed by a wet process such as spin coating, dipping, spray coating, ink-jetting, dispensing, screen printing, offset printing, doctor blade coating, slit coating, roll coating, curtain coating, or knife coating.

Thin films included in the display device can be processed by a photolithography method, for example.

As light used for exposure in the photolithography method, for example, light with an i-line (wavelength: 365 nm), light with a g-line (wavelength: 436 nm), light with an h-line (wavelength: 405 nm), or light in which the i-line, the g-line, and the h-line are mixed can be used. Alternatively, ultraviolet rays, KrF laser light, ArF laser light, or the like can be used. Exposure may be performed by liquid immersion exposure technique. As the light for exposure, extreme ultraviolet (EUV) light or X-rays may also be used. Furthermore, instead of the light used for the exposure, an electron beam can also be used.

For etching of thin films, a dry etching method, a wet etching method, a sandblast method, or the like can be used.

First, as illustrated in FIG. 4A, the insulating layer 171 is formed over a substrate (not illustrated). Next, the conductive layer 172 and a conductive layer 179 are formed over the insulating layer 171, and the insulating layer 173 is formed over the insulating layer 171 so as to cover the conductive layer 172 and the conductive layer 179. Then, the insulating layer 174 is formed over the insulating layer 173, and the insulating layer 175 is formed over the insulating layer 174.

As the substrate, a substrate that has heat resistance high enough to withstand at least heat treatment performed later can be used. For example, it is possible to use a glass substrate; a quartz substrate; a sapphire substrate; a ceramic substrate; an organic resin substrate; or a semiconductor substrate such as a single crystal semiconductor substrate or a polycrystalline semiconductor substrate of silicon, silicon carbide, or the like, a compound semiconductor substrate of silicon germanium or the like, or an SOI substrate.

Next, as illustrated in FIG. 4A, an opening reaching the conductive layer 172 is formed in the insulating layers 175, 174, and 173. Then, the plug 176 is formed to fill the opening.

Subsequently, as illustrated in FIG. 4A, a conductive film 151f to be the conductive layers 151R, 151G, 151B, and 151C and a conductive film 152f to be the conductive layers 152R, 152G, 152B, and 152C are formed over the plug 176 and the insulating layer 175. A metal material can be used for the conductive film 151f, for example. For the conductive film 152f, an oxide including one or more selected from indium, tin, zinc, gallium, titanium, aluminum, and silicon can be used.

Then, as illustrated in FIG. 4A, a resist mask 191 is formed over the conductive film 152f. The resist mask 191 can be formed by application of a photosensitive material (photoresist), light exposure, and development.

Subsequently, as illustrated in FIG. 4B, the conductive films 151f and 152f in a region not overlapping with the resist mask 191 are removed, for example. In this manner, the conductive layers 151 and 152 are formed.

Next, the resist mask 191 is removed as illustrated in FIG. 4C. The resist mask 191 can be removed by ashing using oxygen plasma, for example.

Then, as illustrated in FIG. 4D, an insulating film 156f to be an insulating layer 156R, an insulating layer 156G, an insulating layer 156B, and an insulating layer 156C is formed over the conductive layers 152R, 152G, 152B, and 152C and the insulating layer 175.

As the insulating film 156f, an inorganic insulating film such as an oxide insulating film, a nitride insulating film, an oxynitride insulating film, or a nitride oxide insulating film, e.g., silicon oxynitride, can be used.

Subsequently, as illustrated in FIG. 4E, the insulating film 156f is processed to form the insulating layers 156R, 156G, 156B, and 156C.

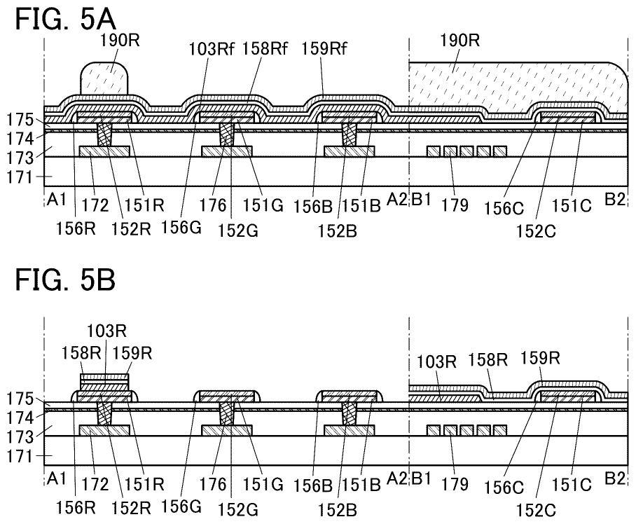

Next, as illustrated in FIG. 5A, an organic compound film 103Rf is formed over the conductive layers 152R, 152G, and 152B and the insulating layer 175. As illustrated in FIG. 5A, the organic compound film 103Rf is not formed over the conductive layer 152C.

Then, as illustrated in FIG. 5A, a sacrificial film 158Rf and a mask film 159Rf are formed.

Providing the sacrificial film 158Rf over the organic compound film 103Rf can reduce damage to the organic compound film 103Rf in the manufacturing process of the display device, resulting in an increase in the reliability of the light-emitting device.

As the sacrificial film 158Rf, a film that is highly resistant to the process conditions for the organic compound film 103Rf, specifically, a film having high etching selectivity with respect to the organic compound film 103Rf is used. For the mask film 159Rf, a film having high etching selectivity with respect to the sacrificial film 158Rf is used.

The sacrificial film 158Rf and the mask film 159Rf are formed at a temperature lower than the upper temperature limit of the organic compound film 103Rf. The typical substrate temperatures in formation of the sacrificial film 158Rf and the mask film 159Rf are each higher than or equal to 100° C. and lower than or equal to 200° C., preferably higher than or equal to 100° C. and lower than or equal to 150° C., further preferably higher than or equal to 100° C. and lower than or equal to 120° C. Since the light-emitting device of one embodiment of the present invention includes the organic compound represented by General Formula (G1) or (G2), a display device having high display quality can be provided even after a heating step performed at higher temperatures.

The sacrificial film 158Rf and the mask film 159Rf are preferably films that can be removed by a wet etching method or a dry etching method.

Note that the sacrificial film 158Rf formed over and in contact with the organic compound film 103Rf is preferably formed by a formation method that is less likely to damage the organic compound film 103Rf than a formation method of the mask film 159Rf. For example, the sacrificial film 158Rf is preferably formed by an ALD method or a vacuum evaporation method rather than a sputtering method.

As each of the sacrificial film 158Rf and the mask film 159Rf, one or more of a metal film, an alloy film, a metal oxide film, a semiconductor film, an organic insulating film, an inorganic insulating film, and the like can be used, for example.

For each of the sacrificial film 158Rf and the mask film 159Rf, it is possible to use a metal material such as gold, silver, platinum, magnesium, nickel, tungsten, chromium, molybdenum, iron, cobalt, copper, palladium, titanium, aluminum, yttrium, zirconium, or tantalum or an alloy material including any of the metal materials, for example. It is particularly preferable to use a low-melting-point material such as aluminum or silver. It is preferable to use a metal material that can block ultraviolet rays for one or both of the sacrificial film 158Rf and the mask film 159Rf, in which case the organic compound film 103Rf can be inhibited from being irradiated with ultraviolet rays in light exposure for patterning and deterioration of the organic compound film 103Rf can be inhibited.

The sacrificial film 158Rf and the mask film 159Rf can each be formed using a metal oxide such as an In—Ga—Zn oxide, an indium oxide, an In—Zn oxide, an In—Sn oxide, an indium titanium oxide (In—Ti oxide), an indium tin zinc oxide (In—Sn—Zn oxide), an indium titanium zinc oxide (In—Ti—Zn oxide), an indium gallium tin zinc oxide (In—Ga—Sn—Zn oxide), or an indium tin oxide including silicon.

In the above metal oxide, in place of gallium, an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used.

The sacrificial film 158Rf and the mask film 159Rf are preferably formed using a semiconductor material such as silicon or germanium for excellent compatibility with a semiconductor manufacturing process. Alternatively, a compound including the above semiconductor material can be used.

As each of the sacrificial film 158Rf and the mask film 159Rf, any of a variety of inorganic insulating films can be used. In particular, an oxide insulating film is preferable because its adhesion to the organic compound film 103Rf is higher than that of a nitride insulating film.

Subsequently, a resist mask 190R is formed as illustrated in FIG. 5A. The resist mask 190R can be formed by application of a photosensitive material (photoresist), light exposure, and development.

The resist mask 190R is provided at a position overlapping with the conductive layer 152R. The resist mask 190R is preferably provided also at a position overlapping with the conductive layer 152C. This can inhibit the conductive layer 152C from being damaged during the process of manufacturing the display device.

Next, as illustrated in FIG. 5B, part of the mask film 159Rf is removed using the resist mask 190R, whereby a mask layer 159R is formed. The mask layer 159R remains over the conductive layers 152R and 152C. After that, the resist mask 190R is removed. Then, part of the sacrificial film 158Rf is removed using the mask layer 159R as a mask (also referred to as a hard mask), whereby a sacrificial layer 158R is formed.

The use of a wet etching method can reduce damage to the organic compound film 103Rf in processing of the sacrificial film 158Rf and the mask film 159Rf, as compared to the case of using a dry etching method. In the case of using a wet etching method, it is preferable to use an alkaline aqueous solution such as a developer or a tetramethylammonium hydroxide (TMAH) aqueous solution, or an acid aqueous solution such as dilute hydrofluoric acid, oxalic acid, phosphoric acid, acetic acid, nitric acid, or a chemical solution including a mixed solution of any of these acids, for example.

In the case of using a dry etching method to process the sacrificial film 158Rf, deterioration of the organic compound film 103Rf can be inhibited by not using a gas including oxygen as the etching gas.

The resist mask 190R can be removed by a method similar to that for the resist mask 191.

Next, as illustrated in FIG. 5B, the organic compound film 103Rf is processed to form the organic compound layer 103R. For example, part of the organic compound film 103Rf is removed using the mask layer 159R and the sacrificial layer 158R as a hard mask, whereby the organic compound layer 103R is formed.

Accordingly, as illustrated in FIG. 5B, the stacked-layer structure of the organic compound layer 103R, the sacrificial layer 158R, and the mask layer 159R remains over the conductive layer 152R. The conductive layers 152G and 152B are exposed.

The organic compound film 103Rf is preferably processed by anisotropic etching. Anisotropic dry etching is particularly preferable. Alternatively, wet etching may be used.

In the case of using a dry etching method, deterioration of the organic compound film 103Rf can be inhibited by not using a gas including oxygen as the etching gas.

A gas including oxygen may be used as the etching gas. When the etching gas includes oxygen, the etching rate can be increased. Thus, the etching can be performed under a low-power condition while an adequately high etching rate is maintained. Accordingly, damage to the organic compound film 103Rf can be reduced. Furthermore, a defect such as attachment of a reaction product generated during the etching can be inhibited.

In the case of using a dry etching method, it is preferable to use a gas including at least one of H₂, CF₄, C₄F₈, SF₆, CHF₃, Cl₂, H₂O, BCl₃, and a Group 18 element such as He or Ar as the etching gas, for example. Alternatively, a gas including oxygen and at least one of the above is preferably used as the etching gas. Alternatively, an oxygen gas may be used as the etching gas.

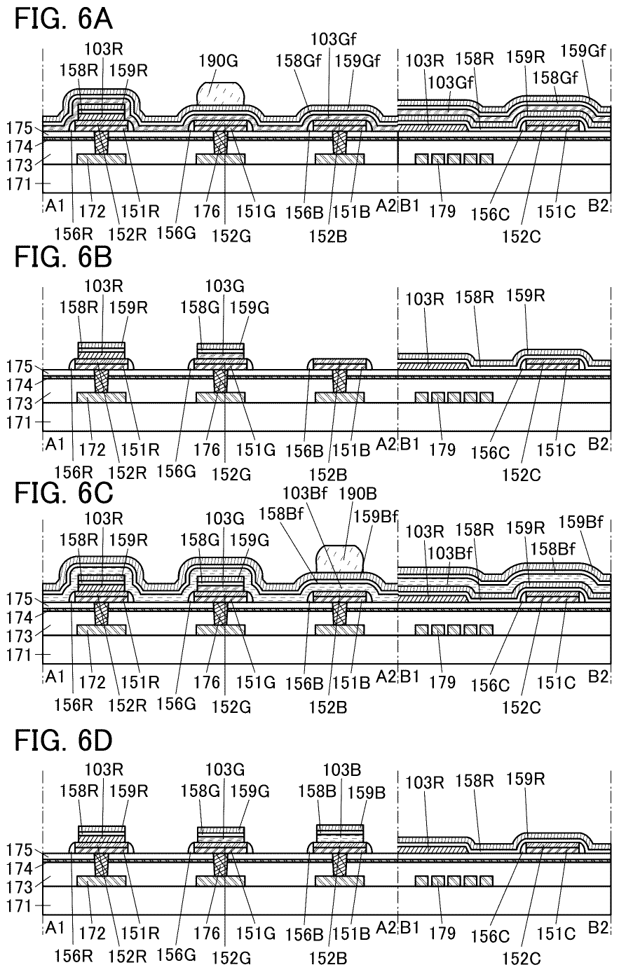

Then, as illustrated in FIG. 6A, an organic compound film 103Gf to be the organic compound layer 103G is formed.

The organic compound film 103Gf can be formed by a method similar to that for forming the organic compound film 103Rf. The organic compound film 103Gf can have a structure similar to that of the organic compound film 103Rf.

Subsequently, a sacrificial film 158Gf and a mask film 159Gf are formed in this order as illustrated in FIG. 6A. After that, a resist mask 190G is formed. The materials and the formation methods of the sacrificial film 158Gf and the mask film 159Gf are similar to those for the sacrificial film 158Rf and the mask film 159Rf. The material and the formation method of the resist mask 190G are similar to those for the resist mask 190R.

The resist mask 190G is provided at a position overlapping with the conductive layer 152G.

Subsequently, as illustrated in FIG. 6B, part of the mask film 159Gf is removed using the resist mask 190G, whereby a mask layer 159G is formed. The mask layer 159G remains over the conductive layer 152G. After that, the resist mask 190G is removed. Then, part of the sacrificial film 158Gf is removed using the mask layer 159G as a mask, whereby the sacrificial layer 158G is formed. Next, the organic compound film 103Gf is processed to form the organic compound layer 103G.

Then, an organic compound film 103Bf is formed as illustrated in FIG. 6C.

The organic compound film 103Bf can be formed by a method similar to that for forming the organic compound film 103Rf. The organic compound film 103Bf can have a structure similar to that of the organic compound film 103Rf.

Subsequently, a sacrificial film 158Bf and a mask film 159Bf are formed in this order as illustrated in FIG. 6C. After that, the resist mask 190B is formed. The materials and the formation methods of the sacrificial film 158Bf and the mask film 159Bf are similar to those for the sacrificial film 158Rf and the mask film 159Rf. The material and the formation method of the resist mask 190B are similar to those for the resist mask 190R.

The resist mask 190B is provided at a position overlapping with the conductive layer 152B.

Subsequently, as illustrated in FIG. 6D, part of the mask film 159Bf is removed using the resist mask 190B, whereby the mask layer 159B is formed. The mask layer 159B remains over the conductive layer 152B. After that, the resist mask 190B is removed. Then, part of the sacrificial film 158Bf is removed using the mask layer 159B as a mask, whereby the sacrificial layer 158B is formed. Next, the organic compound film 103Bf is processed to form the organic compound layer 103B. For example, part of the organic compound film 103Bf is removed using the mask layer 159B and the sacrificial layer 158B as a hard mask, whereby the organic compound layer 103B is formed.

Accordingly, as illustrated in FIG. 6D, the stacked-layer structure of the organic compound layer 103B, the sacrificial layer 158B, and the mask layer 159B remains over the conductive layer 152B. The mask layers 159R and 159G are exposed.

Note that the side surfaces of the organic compound layers 103R, 103G, and 103B are preferably perpendicular or substantially perpendicular to their formation surfaces. For example, the angle between the formation surfaces and these side surfaces is preferably greater than or equal to 600 and less than or equal to 90°.

The distance between two adjacent layers among the organic compound layers 103R, 103G, and 103B, which are formed by a photolithography method as described above, can be reduced to less than or equal to 8 μm, less than or equal to 5 μm, less than or equal to 3 μm, less than or equal to 2 μm, or less than or equal to 1 μm. Here, the distance can be specified, for example, by a distance between facing end portions of two adjacent layers among the organic compound layers 103R, 103G, and 103B. Reducing the distance between the island-shaped organic compound layers makes it possible to provide a display device having high resolution and a high aperture ratio. In addition, the distance between the first electrodes of adjacent light-emitting devices can also be reduced to for example, less than or equal to 10 μm, less than or equal to 8 μm, less than or equal to 5 μm, less than or equal to 3 μm, or less than or equal to 2 μm. Note that the distance between the first electrodes of adjacent light-emitting devices is preferably greater than or equal to 2 μm and less than or equal to 5 μm.

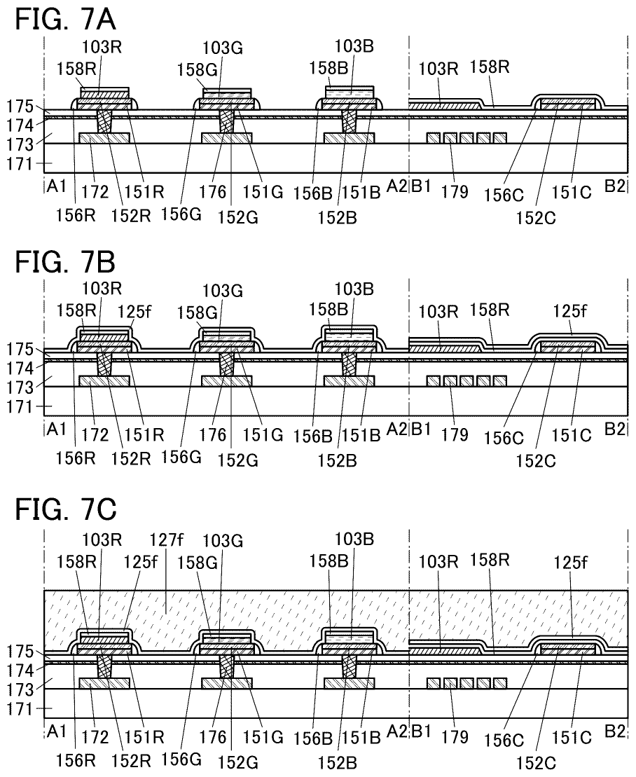

Next, as illustrated in FIG. 7A, the mask layers 159R, 159G, and 159B are preferably removed.

The step of removing the mask layers can be performed by a method similar to that for the step of processing the mask films. Specifically, by using a wet etching method, damage to the organic compound layer 103 at the time of removing the mask layers can be reduced as compared to the case of using a dry etching method.

The mask layers may be removed by being dissolved in a polar solvent such as water or an alcohol. Examples of an alcohol include ethyl alcohol, methyl alcohol, isopropyl alcohol (IPA), and glycerin.

After the mask layers are removed, drying treatment may be performed in order to remove water adsorbed on surfaces. For example, heat treatment in an inert gas atmosphere or a reduced-pressure atmosphere can be performed. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 120° C. The heat treatment is preferably performed in a reduced-pressure atmosphere, in which case drying at a lower temperature is possible.

Next, an inorganic insulating film 125f is formed as illustrated in FIG. 7B.

Then, as illustrated in FIG. 7C, an insulating film 127f to be the insulating layer 127 is formed over the inorganic insulating film 125f.

The substrate temperature at the time of forming the inorganic insulating film 125f and the insulating film 127f is preferably higher than or equal to 60° C., higher than or equal to 80° C., higher than or equal to 100° C., or higher than or equal to 120° C. and lower than or equal to 200° C., lower than or equal to 180° C., lower than or equal to 160° C., lower than or equal to 150° C., or lower than or equal to 140° C.

As the inorganic insulating film 125f, an insulating film having a thickness greater than or equal to 3 nm, greater than or equal to 5 nm, or greater than or equal to 10 nm and less than or equal to 200 nm, less than or equal to 150 nm, less than or equal to 100 nm, or less than or equal to 50 nm is preferably formed in the above-described range of the substrate temperature.

The inorganic insulating film 125f is preferably formed by an ALD method, for example. An ALD method is preferably used, in which case deposition damage is reduced and a film with good coverage can be formed. As the inorganic insulating film 125f, an aluminum oxide film is preferably formed by an ALD method, for example.

The insulating film 127f is preferably formed by the aforementioned wet process. The insulating film 127f is preferably formed by spin coating using a photosensitive material, for example, and specifically preferably formed using a photosensitive resin composition including an acrylic resin.

Then, part of the insulating film 127f is exposed to visible light or ultraviolet rays. The insulating layer 127 is formed in regions that are interposed between any two of the conductive layers 152R, 152G, and 152B and around the conductive layer 152C.

The width of the insulating layer 127 formed later can be controlled with the exposed region of the insulating film 127f. In this embodiment, processing is performed such that the insulating layer 127 includes a portion overlapping with the top surface of the conductive layer 151.

Light used for the exposure preferably includes the i-line (wavelength: 365 nm). Furthermore, light used for the exposure may include at least one of the g-line (wavelength: 436 nm) and the h-line (wavelength: 405 nm).

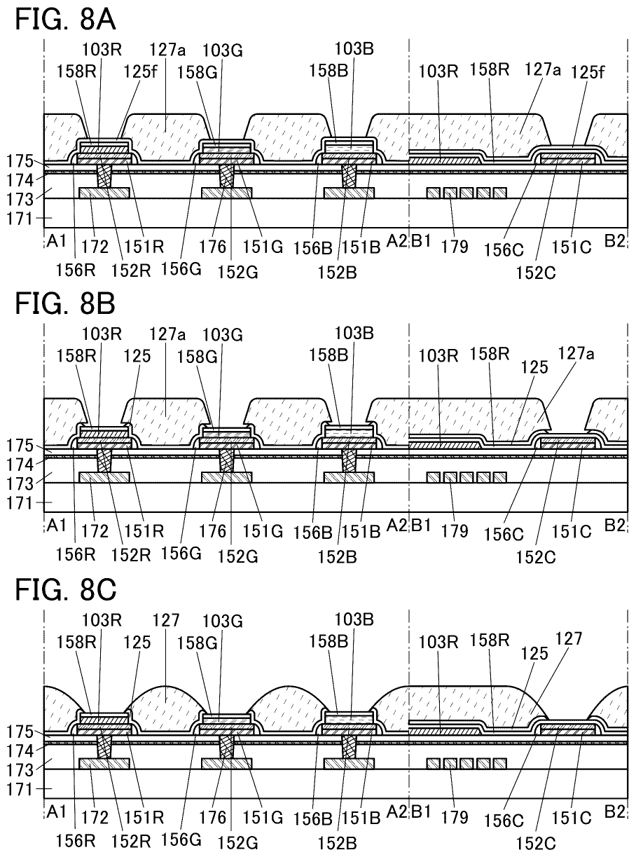

Next, as illustrated in FIG. 8A, development is performed to remove the exposed region of the insulating film 127f, whereby an insulating layer 127a is formed.

Next, as illustrated in FIG. 8B, etching treatment is performed using the insulating layer 127a as a mask to remove part of the inorganic insulating film 125f and reduce the thicknesses of parts of the sacrificial layers 158R, 158G, and 158B. Thus, the inorganic insulating layer 125 is formed under the insulating layer 127a. Moreover, the surfaces of the thin portions in the sacrificial layers 158R, 158G, and 158B are exposed. Note that the etching treatment using the insulating layer 127a as a mask may be hereinafter referred to as first etching treatment.

The first etching treatment can be performed by dry etching or wet etching. Note that the inorganic insulating film 125f is preferably formed using a material similar to that for the sacrificial layers 158R, 158G, and 158B, in which case the first etching treatment can be performed at a time.

In the case of performing dry etching, a chlorine-based gas is preferably used. As the chlorine-based gas, one of Cl₂, BCl₃, SiCl₄, CCl₄, and the like or a mixture of two or more of them can be used. Moreover, one of an oxygen gas, a hydrogen gas, a helium gas, an argon gas, and the like or a mixture of two or more of them can be added as appropriate to the chlorine-based gas. By the dry etching, the thin regions of the sacrificial layers 158R, 158G, and 158B can be formed with favorable in-plane uniformity.

As a dry etching apparatus, a dry etching apparatus including a high-density plasma source can be used. As the dry etching apparatus including a high-density plasma source, an inductively coupled plasma (ICP) etching apparatus can be used, for example. Alternatively, a capacitively coupled plasma (CCP) etching apparatus including parallel plate electrodes can be used.

The first etching treatment is preferably performed by wet etching. The use of wet etching can reduce damage to the organic compound layers 103R, 103G, and 103B, as compared to the case of using dry etching. Wet etching can be performed using an alkaline solution, for example. For instance, TMAH, which is an alkaline solution, can be used for the wet etching of an aluminum oxide film. Alternatively, an acid solution including fluoride can also be used. In this case, puddle wet etching can be performed. Note that the inorganic insulating film 125f is preferably formed using a material similar to that for the sacrificial layers 158R, 158G, and 158B, in which case the first etching treatment can be performed at a time.

The sacrificial layers 158R, 158G, and 158B not be removed completely by the first etching treatment, and the etching treatment be stopped when the thickness of the sacrificial layers 158R, 158G, and 158B is reduced. The sacrificial layers 158R, 158G, and 158B remain over the corresponding organic compound layers 103R, 103G, and 103B in this manner, whereby the organic compound layers 103R, 103G, and 103B can be prevented from being damaged by treatment in a later step.

Next, light exposure is preferably performed on the entire substrate so that the insulating layer 127a is irradiated with visible light or ultraviolet rays. The energy density for the light exposure is preferably greater than 0 mJ/cm² and less than or equal to 800 mJ/cm², further preferably greater than 0 mJ/cm² and less than or equal to 500 mJ/cm². Performing such light exposure after the development can sometimes increase the degree of transparency of the insulating layer 127a. In addition, it is sometimes possible to lower the substrate temperature required for subsequent heat treatment for changing the shape of the insulating layer 127a into a tapered shape.

Here, when a barrier insulating layer against oxygen (e.g., an aluminum oxide film) is provided as each of the sacrificial layers 158R, 158G, and 158B, diffusion of oxygen to the organic compound layers 103R, 103G, and 103B can be inhibited.

Then, heat treatment (also referred to as post-baking) is performed. The heat treatment can change the insulating layer 127a into the insulating layer 127 having a tapered side surface (FIG. 8C). The heat treatment is performed at a temperature lower than the upper temperature limit of the organic compound layer. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 130° C. The heating atmosphere may be an air atmosphere or an inert gas atmosphere. Moreover, the heating atmosphere may be an atmospheric-pressure atmosphere or a reduced-pressure atmosphere. Accordingly, adhesion between the insulating layer 127 and the inorganic insulating layer 125 can be improved, and corrosion resistance of the insulating layer 127 can be increased.

When the sacrificial layers 158R, 158G, and 158B are not completely removed by the first etching treatment and the thinned sacrificial layers 158R, 158G, and 158B are left, the organic compound layers 103R, 103G, and 103B can be prevented from being damaged and deteriorating in the heat treatment. This can increase the reliability of the light-emitting device.

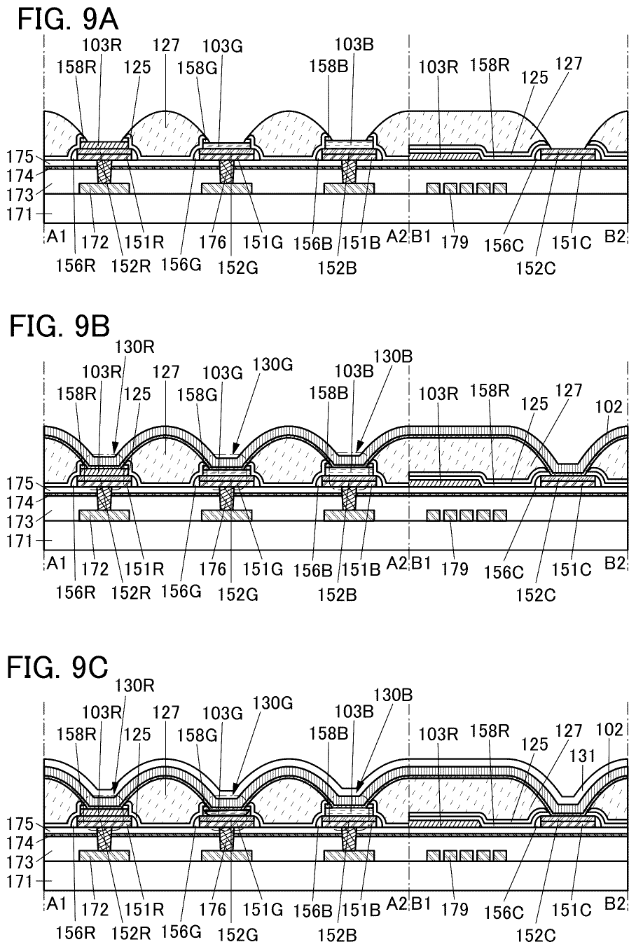

Next, as illustrated in FIG. 9A, etching treatment is performed using the insulating layer 127 as a mask to remove parts of the sacrificial layers 158R, 158G, and 158B. Thus, openings are formed in the sacrificial layers 158R, 158G, and 158B, and the top surfaces of the organic compound layers 103R, 103G, and 103B and the conductive layer 152C are exposed. Note that this etching treatment may be hereinafter referred to as second etching treatment.

An end portion of the inorganic insulating layer 125 is covered with the insulating layer 127. FIG. 9A illustrates an example where part of the end portion of the sacrificial layer 158G (specifically a tapered portion formed by the first etching treatment) is covered with the insulating layer 127 and a tapered portion formed by the second etching treatment is exposed.

The second etching treatment is performed by wet etching. The use of wet etching can reduce damage to the organic compound layers 103R, 103G, and 103B, as compared to the case of using dry etching. Wet etching can be performed using an alkaline solution or an acid solution, for example. An aqueous solution is preferably used in order that the organic compound layer 103 is not dissolved.

Next, as illustrated in FIG. 9B, a common electrode (the second electrode 102) is formed over the organic compound layers 103R, 103G, and 103B, the conductive layer 152C, and the insulating layer 127. The common electrode (the second electrode 102) can be formed by a sputtering method, a vacuum evaporation method, or the like.

Next, as illustrated in FIG. 9C, the protective layer 131 is formed over the common electrode (the second electrode 102). The protective layer 131 can be formed by a vacuum evaporation method, a sputtering method, a CVD method, an ALD method, or the like. The protective layer 131 can also serve as the cap layer. Providing the cap layer can improve light extraction efficiency in the case of a top-emission light-emitting device. For example, with the use of a material with an ordinary refractive index (nₒ) of higher than or equal to 1.90 at a wavelength of 450 nm, a material with an ordinary refractive index (nₒ) of higher than or equal to 1.80 at a wavelength of 520 nm, or a material with an ordinary refractive index (nₒ) of higher than or equal to 1.75 at a wavelength of 630 nm, the total reflection of light from the organic compound layer 103 by the cap layer can be inhibited, leading to an improvement in light extraction efficiency. The cap layer can also serve as a protective layer.

In order to prevent air exposure of the light-emitting device that has yet to be incorporated in a display device or a light-emitting apparatus, a sealing film may be provided over the protective layer 131. The sealing film can be formed using a material that is less likely to transmit impurities such as water easily. Specifically, an aluminum oxide film is preferably provided by an ALD method. Note that in order to prevent air exposure of the light-emitting device in which the sealing film has yet to be provided after the protective layer 131 is formed, the light-emitting device is preferably transferred into an ALD apparatus in a glove box with a nitrogen atmosphere after the protective layer 131 is formed. In that case, the oxygen concentration in the glove box is preferably lower than or equal to 100 ppm, further preferably lower than or equal to 10 ppm, still further preferably lower than or equal to 1 ppm.

Then, the substrate 120 is bonded to the protective layer 131 using the resin layer 122, so that the display device can be manufactured. In the method for manufacturing the display device of one embodiment of the present invention, the insulating layer 156 is formed to include a region overlapping with the side surface of the conductive layer 151 and the conductive layer 152 is formed to cover the conductive layer 151 and the insulating layer 156 as described above. This can increase the yield of the display device and inhibit generation of defects. In addition, a microlens array is provided over the protective layer 131 or the sealing film before bonding of the substrate 120, and then the substrate 120 is bonded, whereby a display device including the microlens array can be manufactured.

As described above, in the method for manufacturing the display device of one embodiment of the present invention, the island-shaped organic compound layers 103R, 103G, and 103B are formed not by using a fine metal mask but by processing a film formed on the entire surface; thus, the island-shaped layers can be formed to have a uniform thickness. In addition, a high-resolution display device or a display device with a high aperture ratio can be obtained. Furthermore, even when the resolution or the aperture ratio is high and the distance between the subpixels is extremely short, the organic compound layers 103R, 103G, and 103B can be inhibited from being in contact with each other in the adjacent subpixels. As a result, generation of a leakage current between the subpixels can be inhibited. This can prevent crosstalk, so that a display device with extremely high contrast can be obtained. Moreover, even a display device that includes tandem light-emitting devices formed by a photolithography method can have favorable characteristics.

### Embodiment 5

In this embodiment, a display device of one embodiment of the present invention will be described.

The display device in this embodiment can be a high-resolution display device. Thus, the display device in this embodiment can be used for display portions of information terminals (wearable devices) such as watch-type and bracelet-type information terminals and display portions of wearable devices capable of being worn on a head, such as a VR device like a head mounted display (HMD) and a glasses-type AR device.

The display device in this embodiment can be a high-definition display device or a large-sized display device. Accordingly, the display device in this embodiment can be used for display portions of a digital camera, a digital video camera, a digital photo frame, a mobile phone, a portable game console, a portable information terminal, and an audio reproducing device, in addition to display portions of electronic appliances with a relatively large screen, such as a television device, desktop and laptop personal computers, a monitor of a computer and the like, digital signage, and a large game machine such as a pachinko machine.

### [Display Module]

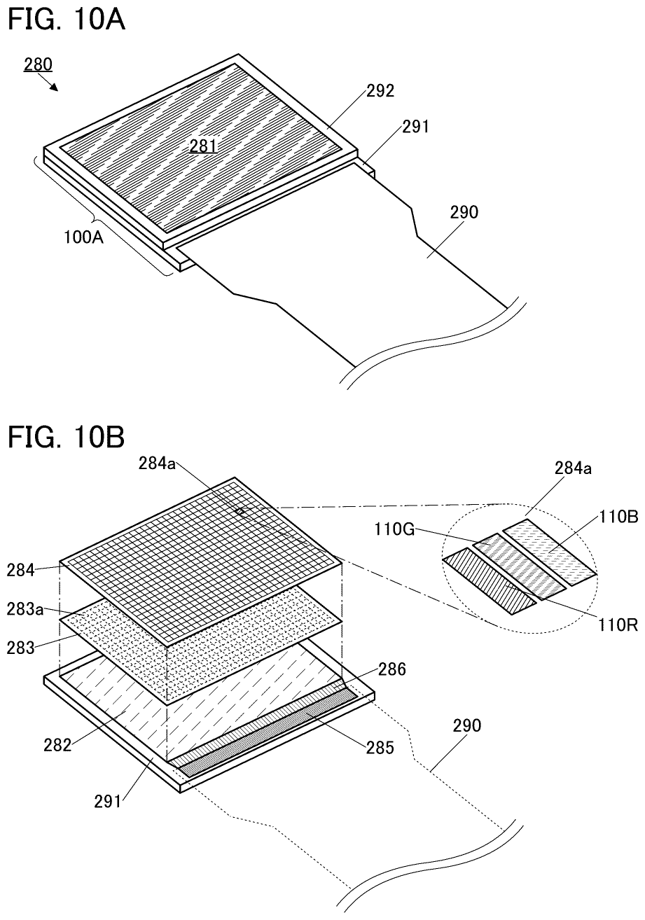

FIG. 10A is a perspective view of a display module 280. The display module 280 includes a display device 100A and an FPC 290. Note that the display device included in the display module 280 is not limited to the display device 100A and may be any of display devices 100B to 100E described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 10B is a perspective view schematically showing the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is provided in a portion over the substrate 291 that does not overlap with the pixel portion 284. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 10B. The pixels 284a can employ any of the structures described in the above embodiments.

The pixel circuit portion 283 includes a plurality of pixel circuits 283a arranged periodically.

One pixel circuit 283a is a circuit that controls driving of a plurality of elements included in one pixel 284a.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high.

Such a display module 280 has extremely high resolution, and thus can be suitably used for a VR device such as an HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic appliances including a relatively small display portion.

### [Display Device 100A]

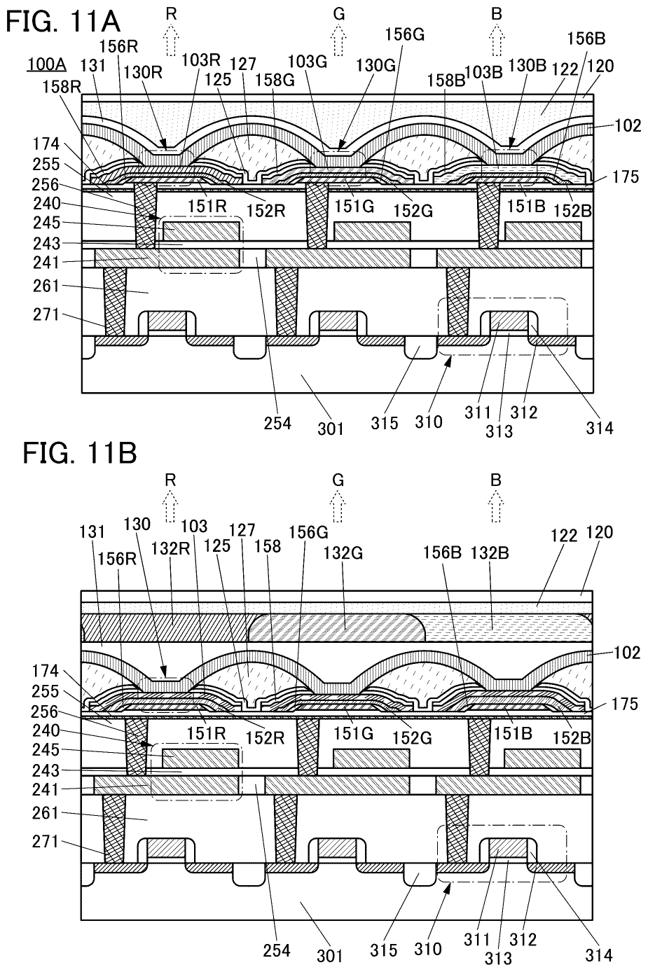

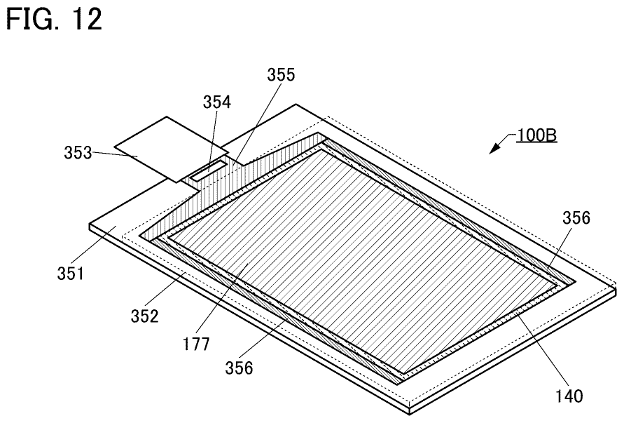

The display device 100A illustrated in FIG. 11A includes a substrate 301, the light-emitting devices 130R, 130G, and 130B, a capacitor 240, and a transistor 310.

The substrate 301 corresponds to the substrate 291 in FIGS. 10A and 10B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of a source and a drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. An insulator is provided in regions between adjacent light-emitting devices.

The insulating layer 156R is provided to include a region overlapping with the side surface of the conductive layer 151R. The insulating layer 156G is provided to include a region overlapping with the side surface of the conductive layer 151G. The insulating layer 156B is provided to include a region overlapping with the side surface of the conductive layer 151B. The conductive layer 152R is provided to cover the conductive layer 151R and the insulating layer 156R. The conductive layer 152G is provided to cover the conductive layer 151G and the insulating layer 156G. The conductive layer 152B is provided to cover the conductive layer 151B and the insulating layer 156B. The sacrificial layer 158R is positioned over the organic compound layer 103R. The sacrificial layer 158G is positioned over the organic compound layer 103G. The sacrificial layer 158B is positioned over the organic compound layer 103B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. Any of a variety of conductive materials can be used for the plugs.

The protective layer 131 is provided over the light-emitting devices 130R, 130G, and 130B. The substrate 120 is bonded to the protective layer 131 with the resin layer 122. Embodiment 4 can be referred to for the details of the light-emitting device 130 and the components thereover up to the substrate 120. The substrate 120 corresponds to the substrate 292 in FIG. 10A.

FIG. 11B shows a variation example of the display device 100A shown in FIG. 11A. The display device shown in FIG. 11B includes a coloring layer 132R, a coloring layer 132G, and a coloring layer 132B, and each of the light-emitting devices 130 includes a region overlapping with one of the coloring layers 132R, 132G, and 132B. In the display device shown in FIG. 11B, the light-emitting device 130 can emit white light, for example. The coloring layer 132R, the coloring layer 132G, and the coloring layer 132B can transmit red light, green light, and blue light, respectively, for example.

### [Display Device 100B]

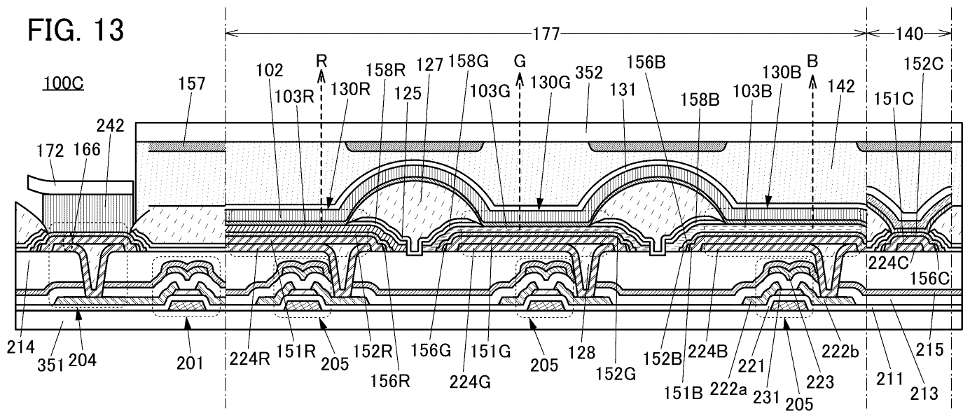

FIG. 12 is a perspective view of the display device 100B, and FIG. 13 is a cross-sectional view of the display device 100C.

In the display device 100B, a substrate 352 and a substrate 351 are bonded to each other. In FIG. 12, the substrate 352 is denoted by a dashed line.

The display device 100B includes the pixel portion 177, the connection portion 140, a circuit 356, a wiring 355, and the like. FIG. 12 illustrates an example where an IC 354 and an FPC 353 are mounted on the display device 100B. Thus, the structure illustrated in FIG. 12 can be regarded as a display module including the display device 100B, the integrated circuit (IC), and the FPC. Here, a display device in which a substrate is equipped with a connector such as an FPC or mounted with an IC is referred to as a display module.

The connection portion 140 is provided outside the pixel portion 177. The number of connection portions 140 may be one or more. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 12 illustrates an example where the IC 354 is provided over the substrate 351 by a chip on glass (COG) method, a chip on film (COF) method, or the like. An IC including a scan line driver circuit, a signal line driver circuit, or the like can be used as the IC 354, for example. Note that the display device 100B and the display module are not necessarily provided with an IC. Alternatively, the IC may be mounted on the FPC by a COF method, for example.

FIG. 13 illustrates the display device 100C in an example of cross sections of part of a region including the FPC 353, part of the circuit 356, part of the pixel portion 177, part of the connection portion 140, and part of a region including an end portion of the display device 100B in FIG. 12.

### [Display Device 100C]

The display device 100C illustrated in FIG. 13 includes a transistor 201, a transistor 205, the light-emitting device 130R that emits red light, the light-emitting device 130G that emits green light, the light-emitting device 130B that emits blue light, and the like between the substrate 351 and the substrate 352.

Embodiment 4 can be referred to for the details of the light-emitting devices 130R, 130G, and 130B.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B.

The conductive layer 224R is connected to a conductive layer 222b included in the transistor 205 through an opening provided in an insulating layer 214. An end portion of the conductive layer 151R is positioned outward from an end portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G, and the insulating layer 156G in the light-emitting device 130G are not described in detail because they are respectively similar to the conductive layers 224R, 151R, and 152R, and the insulating layer 156R in the light-emitting device 130R; the same applies to the conductive layers 224B, 151B, and 152B, and the insulating layer 156B in the light-emitting device 130B.

The conductive layers 224R, 224G, and 224B each have a depressed portion covering the opening provided in the insulating layer 214. A layer 128 is embedded in the depressed portion.

The layer 128 has a function of filling the depressed portions of the conductive layers 224R, 224G, and 224B to obtain planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping with the depressed portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The protective layer 131 is provided over the light-emitting devices 130R, 130G, and 130B. The protective layer 131 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 13, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided in a frame shape not to overlap with the light-emitting device. Furthermore, the space may be filled with a resin different from that used for the frame-shaped adhesive layer 142.

FIG. 13 illustrates an example where the connection portion 140 includes a conductive layer 224C obtained by processing the same conductive film as the conductive layers 224R, 224G, and 224B; the conductive layer 151C obtained by processing the same conductive film as the conductive layers 151R, 151G, and 151B; and the conductive layer 152C obtained by processing the same conductive film as the conductive layers 152R, 152G, and 152B. In the example illustrated in FIG. 13, the insulating layer 156C is provided to include a region overlapping with the side surface of the conductive layer 151C.

The display device 100C has a top-emission structure. Light from the light-emitting device is emitted toward the substrate 352. For the substrate 352, a material with a high visible-light-transmitting property is preferably used. The pixel electrode includes a material that reflects visible light, and the common electrode (the second electrode 102) opposite thereto includes a material that transmits visible light.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

An inorganic insulating film is preferably used as each of the insulating layers 211, 213, and 215.

An organic insulating layer is suitable as the insulating layer 214 functioning as a planarization layer.

Each of the transistors 201 and 205 includes a conductive layer 221 functioning as a gate, the insulating layer 211 functioning as the gate insulating layer, a conductive layer 222a and the conductive layer 222b functioning as a source and a drain, a semiconductor layer 231, the insulating layer 213 functioning as the gate insulating layer, and a conductive layer 223 functioning as a gate.

A connection portion 204 is provided in a region of the substrate 351 not overlapping with the substrate 352. In the connection portion 204, one of the source electrode and the drain electrode of the transistor 201 is electrically connected to the FPC 353 through a conductive layer 166 and a connection layer 242. As an example, the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

The light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

As the connection layer 242, an anisotropic conductive film (ACF), an anisotropic conductive paste (ACP), or the like can be used.

### [Display Device 100D]

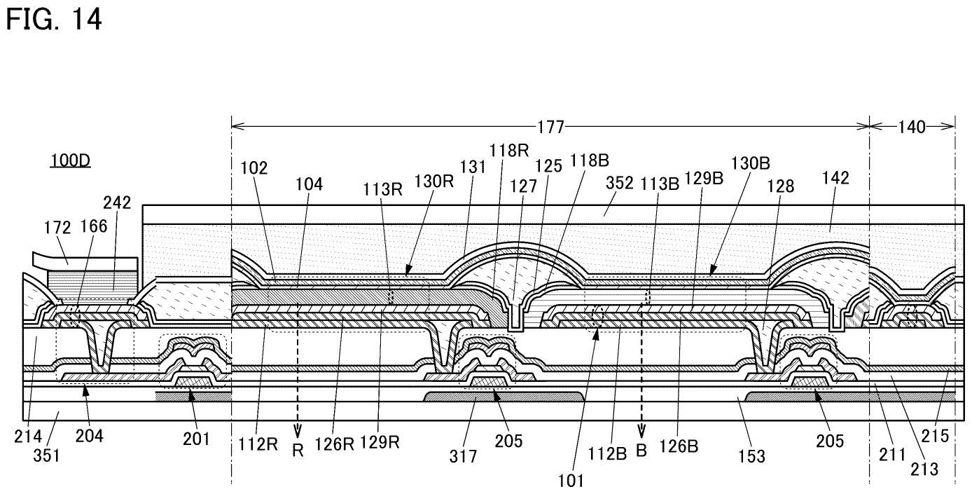

The display device 100D illustrated in FIG. 14 differs from the display device 100C illustrated in FIG. 13 mainly in having a bottom-emission structure.

Light from the light-emitting device is emitted toward the substrate 351. For the substrate 351, a material with a high visible-light-transmitting property is preferably used. By contrast, there is no limitation on the light-transmitting property of a material used for the substrate 352.

A light-blocking layer 317 is preferably formed between the substrate 351 and the transistor 201 and between the substrate 351 and the transistor 205. FIG. 14 illustrates an example where the light-blocking layer 317 is provided over the substrate 351, an insulating layer 153 is provided over the light-blocking layer 317, and the transistors 201 and 205 and the like are provided over the insulating layer 153.

The light-emitting device 130R includes a conductive layer 112R, a conductive layer 126R over the conductive layer 112R, and a conductive layer 129R over the conductive layer 126R.

The light-emitting device 130B includes a conductive layer 112B, a conductive layer 126B over the conductive layer 112B, and a conductive layer 129B over the conductive layer 126B.

A material with a high visible-light-transmitting property is used for each of the conductive layers 112R, 112B, 126R, 126B, 129R, and 129B. A material that reflects visible light is preferably used for the second electrode 102.

Although not illustrated in FIG. 14, the light-emitting device 130G is also provided.

Although FIG. 14 and the like illustrate an example where the top surface of the layer 128 includes a flat portion, the shape of the layer 128 is not particularly limited.

### [Display Device 100D2]

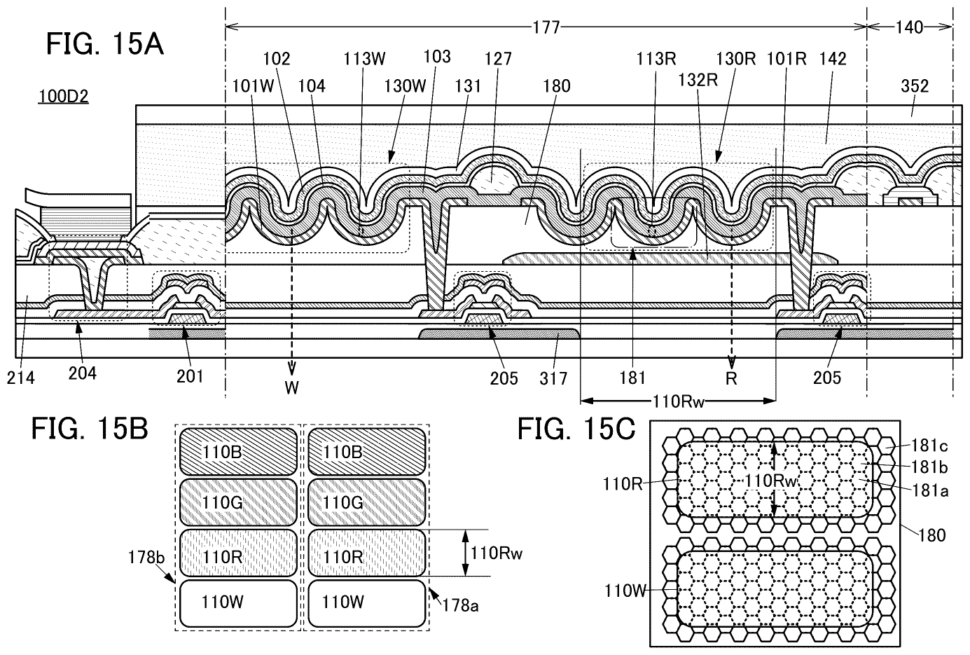

The display device 100D2 illustrated in FIG. 15A is an example of a bottom-emission display device different from the display device 100D illustrated in FIG. 14. The display device 100D2 is different from the display device 100D in including an organic resin layer 180. Note that the reference numerals of the components that are the same as those in FIG. 14 are sometimes omitted and the description for FIG. 14 is referred to for the details of such components.

FIG. 15B is a top-view layout of the pixel 178 (pixels 178a and 178b) including the subpixel 110 (the subpixels 110R, 110G, and 110B and a subpixel 110W), and FIG. 15C is a top view of the organic resin layer 180 in a region where the subpixels 110R and 110G included in the pixel 178 are formed. A region of the subpixel 110R between the light-blocking layers 317 can be represented as a width 110Rw in a light-emitting region.

As illustrated in FIG. 15A, the organic resin layer 180 is provided over the insulating layer 214. As illustrated in FIG. 15C and the region surrounded by the dashed-dotted line in FIG. 15A, the organic resin layer 180 includes a depressed portion 181 (depressed portions 181a and 181b) having a curved surface at least in a region where the subpixel is formed. Note that the depressed portion 181 may be provided outside the light-emitting region, like a depressed portion 181c. With the depressed portion 181c, light emission caused in a region overlapping with the light-blocking layer 317 or light travelled into the region overlapping with the light-blocking layer 317 can be refracted and extracted from the light-emitting region, whereby light emission efficiency can be improved.

A plurality of depressed portions 181 may be formed in a matrix. The depressed portions 181a and 181b may be provided in contact with each other or may be provided to have a flat surface therebetween.

In FIGS. 15A to 15C, although the top surface shape and the cross-sectional shape of the depressed portion are hexagonal (FIG. 15C) and semicircular (FIG. 15A), respectively, other shapes may be employed as needed. Examples of the top surface shape of the depressed portion include polygons such as a triangle, a tetragon (including a rectangle and a square), and a pentagon; polygons with rounded corners; an ellipse; and a circle.

An insulating layer including an organic material can be used as the organic resin layer 180. Examples of materials used for the organic resin layer 180 include an acrylic resin, a polyimide resin, an epoxy resin, an imide resin, a polyamide resin, a polyimide-amide resin, a silicone resin, a siloxane resin, a benzocyclobutene-based resin, a phenol resin, and precursors of these resins. The organic resin layer 180 may be formed using an organic material such as polyvinyl alcohol (PVA), polyvinyl butyral, polyvinylpyrrolidone, polyethylene glycol, polyglycerin, pullulan, water-soluble cellulose, or an alcohol-soluble polyamide resin.

A photosensitive resin can also be used for the organic resin layer 180. A photoresist may be used for the photosensitive resin. As the photosensitive resin, a positive photosensitive material or a negative photosensitive material can be used.

The organic resin layer 180 may include a material absorbing visible light. For example, the organic resin layer 180 itself may be made of a material absorbing visible light, or the organic resin layer 180 may include a pigment absorbing visible light. For example, the organic resin layer 180 can be formed using a resin that can be used as a color filter transmitting red, blue, or green light and absorbing light of the other colors; or a resin that includes carbon black as a pigment and functions as a black matrix.

The first electrode 101 (the first electrode 101R and a first electrode 101W) is provided over the organic resin layer 180 and the organic compound layer 103 is provided over the first electrode 101. End portions of the first electrode 101 and the organic compound layer 103 may be covered with the insulating layer 127.

The first electrode 101 formed over the organic resin layer 180 also has a depressed portion along the depressed portion of the organic resin layer 180. The organic compound layer 103 formed over the first electrode 101 also has a depressed portion along the depressed portion of the first electrode 101. The common layer 104 formed over the organic compound layer 103 also has a depressed portion along the depressed portion of the organic compound layer 103. The second electrode 102 formed over the common layer 104 also has a depressed portion along the depressed portion of the common layer 104. That is, the depressed portions of the organic resin layer 180, the first electrode 101, the organic compound layer 103, the common layer 104, and the second electrode 102 overlap with each other.

The common layer 104 is provided over the organic compound layer 103 and the insulating layer 127, and the second electrode 102 is provided over the common layer 104. The protective layer 131 is provided over the second electrode 102 and bonded to the substrate 352 with the adhesive layer 142 therebetween.

Although FIGS. 15A to 15C illustrate a light-emitting device 130W and the light-emitting device 130R and does not illustrate the light-emitting devices 130G and 130B, the light-emitting devices 130G and 130B are also provided.

### [Display Device 100E]

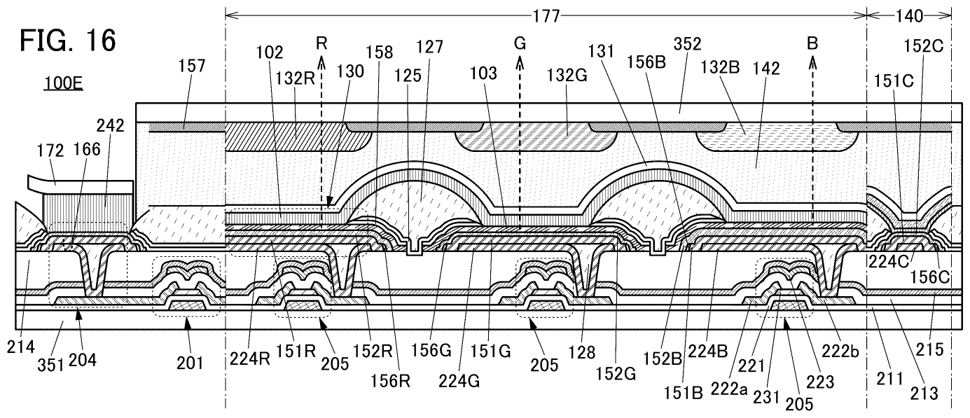

The display device 100E illustrated in FIG. 16 is a variation example of the display device 100C illustrated in FIG. 13 and differs from the display device 100C mainly in including the coloring layers 132R, 132G, and 132B.

In the display device 100E, the light-emitting device 130 includes a region overlapping with one of the coloring layers 132R, 132G, and 132B. The coloring layers 132R, 132G, and 132B can be provided on a surface of the substrate 352 on the substrate 351 side. End portions of the coloring layers 132R, 132G, and 132B can overlap with the light-blocking layer 157.

In the display device 100E, the light-emitting device 130 can emit white light, for example. The coloring layer 132R, the coloring layer 132G, and the coloring layer 132B can transmit red light, green light, and blue light, respectively, for example. Note that in the display device 100E, the coloring layers 132R, 132G, and 132B may be provided between the protective layer 131 and the adhesive layer 142.

### [Display Device 100E2]

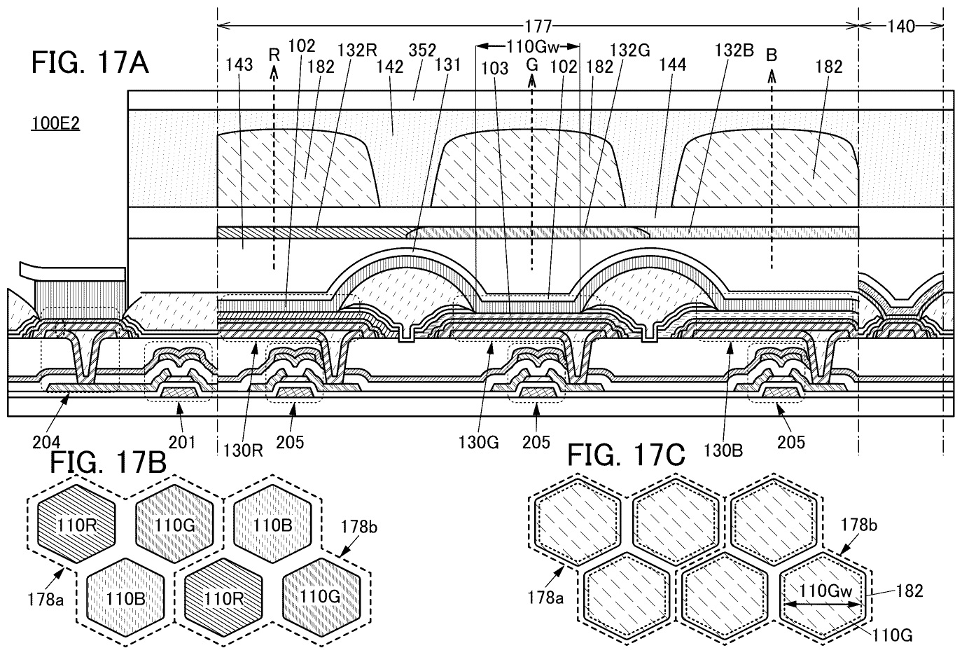

A display device 100E2 illustrated in FIG. 17A is a variation example of the display device 100E illustrated in FIG. 16 and includes microlenses 182 over the coloring layers 132R, 132G, and 132B. Note that the reference numerals of the components that are the same as those in FIG. 16 are sometimes omitted and the description for FIG. 16 is referred to for the details of such components.

FIG. 17B is a top-view layout of the pixel 178 (the pixels 178a and 178b) including the subpixel 110 (the subpixels 110R, 110G, and 110B), and FIG. 17C is a top view of the microlens 182 in a region where the subpixels 110R and 110G included in the pixel 178 are formed. A region of the subpixel 110G where the common electrode (the second electrode 102) and the organic compound layer 103 are in contact with each other can be represented as a width 110Gw in a light-emitting region.

In the display device 100E2 illustrated in FIG. 17A, a planarization film 143 is provided over the protective layer 131, and the coloring layers 132R, 132G, and 132B are provided over a planarization film 143. The planarization film 144 is provided to cover the coloring layers 132R, 132G, and 132B. The microlenses 182 are provided over the planarization film 144.

Note that as illustrated in FIG. 17C, the microlens 182 is preferably provided for each of the subpixels in a region where the subpixel is formed.

Although the top surface shape of the microlens 182 is illustrated as a hexagon in FIG. 17C, other shapes may be employed as needed. Examples of the top surface shape of the depressed portion include polygons such as a triangle, a tetragon (including a rectangle and a square), and a pentagon; polygons with rounded corners; an ellipse; and a circle.

The microlens 182 can be formed using a material similar to that for the organic resin layer 180.

This embodiment can be combined as appropriate with the other embodiments or the examples. In this specification, in the case where a plurality of structure examples are illustrated in one embodiment, the structure examples can be combined as appropriate.

### Embodiment 6

In this embodiment, electronic appliances of embodiments of the present invention will be described.

Electronic appliances of this embodiment include the display device of one embodiment of the present invention in their display portions. The display device of one embodiment of the present invention has low power consumption and high reliability. Thus, the display device of one embodiment of the present invention can be used for display portions of a variety of electronic appliances.

Examples of the electronic appliances include a digital camera, a digital video camera, a digital photo frame, a mobile phone, a portable game console, a portable information terminal, and an audio reproducing device, in addition to electronic appliances with a relatively large screen, such as a television device, desktop and notebook personal computers, a monitor of a computer and the like, digital signage, and a large game machine such as a pachinko machine.

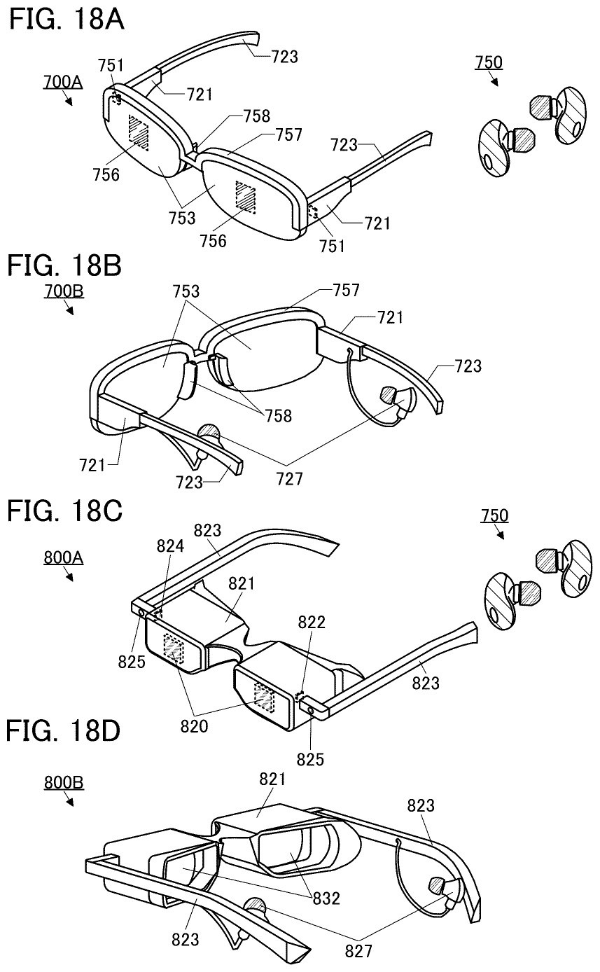

Examples of wearable devices capable of being worn on a head are described with reference to FIGS. 18A to 18D.

An electronic appliance 700A illustrated in FIG. 18A and an electronic appliance 700B illustrated in FIG. 18B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display device of one embodiment of the present invention can be used for the display panels 751. Thus, a highly reliable electronic appliance is obtained.

The electronic appliances 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753.

In the electronic appliances 700A and 700B, a camera capable of capturing images of the front side may be provided as the image capturing portion. Furthermore, when the electronic appliances 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic appliances 700A and 700B are provided with a battery, so that they can be charged wirelessly and/or by wire.

A touch sensor module may be provided in the housing 721.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

An electronic appliance 800A illustrated in FIG. 18C and an electronic appliance 800B illustrated in FIG. 18D each include a pair of display portions 820, a housing 821, a communication portion 822, a pair of wearing portions 823, a control portion 824, a pair of image capturing portions 825, and a pair of lenses 832.

The display device of one embodiment of the present invention can be used in the display portions 820. Thus, a highly reliable electronic appliance is obtained.

The display portions 820 are positioned inside the housing 821 so as to be seen through the lenses 832. When the pair of display portions 820 display different images, three-dimensional display using parallax can be performed.

The electronic appliances 800A and 800B preferably include a mechanism for horizontally adjusting the positions of the lenses 832 and the display portions 820 so that the lenses 832 and the display portions 820 are located at positions optimal for the positions of the user's eyes.

The electronic appliance 800A or the electronic appliance 800B can be mounted on the user's head with the wearing portions 823.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to cover a plurality of angles of view, such as a telephoto angle of view and a wide-angle of view.

The electronic appliance 800A may include a vibration mechanism that functions as bone-conduction earphones.

The electronic appliances 800A and 800B may each include an input terminal. To the input terminal, a cable for supplying a video signal from a video output device or the like, power for charging a battery provided in the electronic appliance, and the like can be connected.

The electronic appliance of one embodiment of the present invention may have a function of performing wireless communication with earphones 750.

The electronic appliance may include an earphone portion. The electronic appliance 700B illustrated in FIG. 18B includes earphone portions 727. Part of a wiring that connects the earphone portion 727 and the control portion may be positioned inside the housing 721 or the wearing portion 723.

Similarly, the electronic appliance 800B illustrated in FIG. 18D includes earphone portions 827. For example, the earphone portion 827 can be connected to the control portion 824 by wire.

As described above, both the glasses-type device (e.g., the electronic appliances 700A and 700B) and the goggles-type device (e.g., the electronic appliances 800A and 800B) are preferable as the electronic appliance of one embodiment of the present invention.

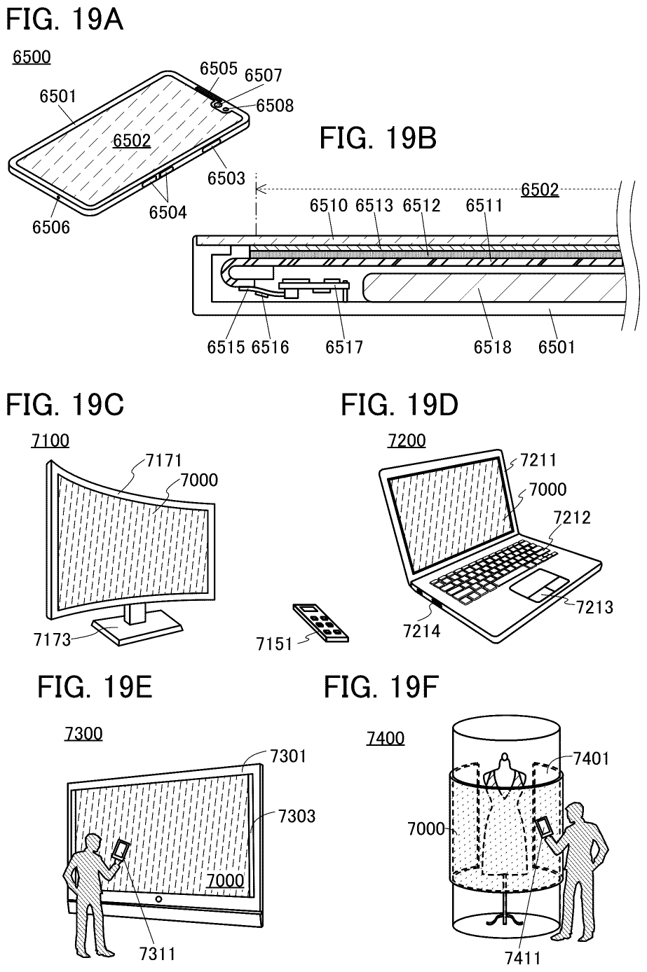

An electronic appliance 6500 illustrated in FIG. 19A is a portable information terminal that can be used as a smartphone.

The electronic appliance 6500 includes a housing 6501, a display portion 6502, a power button 6503, buttons 6504, a speaker 6505, a microphone 6506, a camera 6507, a light source 6508, and the like. The display portion 6502 has a touch panel function.

The display device of one embodiment of the present invention can be used in the display portion 6502. Thus, a highly reliable electronic appliance is obtained.

FIG. 19B is a schematic cross-sectional view including an end portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with a bonding layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The display device of one embodiment of the present invention can be used in the display panel 6511. Thus, an extremely lightweight electronic appliance can be achieved. Since the display panel 6511 is extremely thin, the battery 6518 with high capacity can be mounted without an increase in the thickness of the electronic appliance. Moreover, part of the display panel 6511 is folded back so that a connection portion with the FPC 6515 is provided on the back side of the pixel portion, whereby an electronic appliance with a narrow bezel can be achieved.

FIG. 19C illustrates an example of a television device. In a television device 7100, a display portion 7000 is incorporated in a housing 7171. Here, the housing 7171 is supported by a stand 7173.

The display device of one embodiment of the present invention can be used in the display portion 7000. Thus, a highly reliable electronic appliance is obtained.

Operation of the television device 7100 illustrated in FIG. 19C can be performed with an operation switch provided in the housing 7171 and a separate remote control 7151.

FIG. 19D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The display device of one embodiment of the present invention can be used in the display portion 7000. Thus, a highly reliable electronic appliance is obtained.

FIGS. 19E and 19F show examples of digital signage that can be used for store windows, showcases, and the like.

Digital signage 7300 illustrated in FIG. 19E includes a housing 7301, the display portion 7000, a speaker 7303, and the like. The digital signage 7300 can also include an LED lamp, an operation key (including a power switch or an operation switch), a connection terminal, a variety of sensors, a microphone, and the like.

FIG. 19F illustrates digital signage 7400 attached to a cylindrical pillar 7401. The digital signage 7400 includes the display portion 7000 provided along a curved surface of the pillar 7401.

In FIGS. 19E and 19F, the display device of one embodiment of the present invention can be used in the display portion 7000. Thus, a highly reliable electronic appliance is obtained.

A larger area of the display portion 7000 can increase the amount of information that can be provided at a time. The larger display portion 7000 attracts more attention, so that the effectiveness of the advertisement can be increased, for example.

Specifically, in the case where the display device of one embodiment of the present invention is used for the digital signage 7400 illustrated in FIGS. 19E and 19F and the like that displays advertisements and the like, the display device being a light-transmitting panel can increase the flexibility of representation in advertising. A light-transmitting display device can be manufactured, for example, by using a wiring and a support member each of which is formed of a conductive film that transmits visible light and adjusting the distance between pixel electrodes. When the pillar 7401 is formed of tempered glass or the like, the pillar 7401 can also be used as a show case.

The tandem light-emitting device of one embodiment of the present invention in addition to the wiring and the support member each of which is formed of the conductive film that transmits visible light can increase the luminance per pixel. That is, favorable display can be performed even when the aperture ratio of the display device is decreased; thus, the light-transmitting property of the display portion of the display device can be increased. Accordingly, such a structure is suitably used in the light-transmitting display device of one embodiment of the present invention.

As illustrated in FIGS. 19E and 19F, it is preferable that the digital signage 7300 or the digital signage 7400 can work with an information terminal 7311 or an information terminal 7411, such as a smartphone that a user has, through wireless communication.

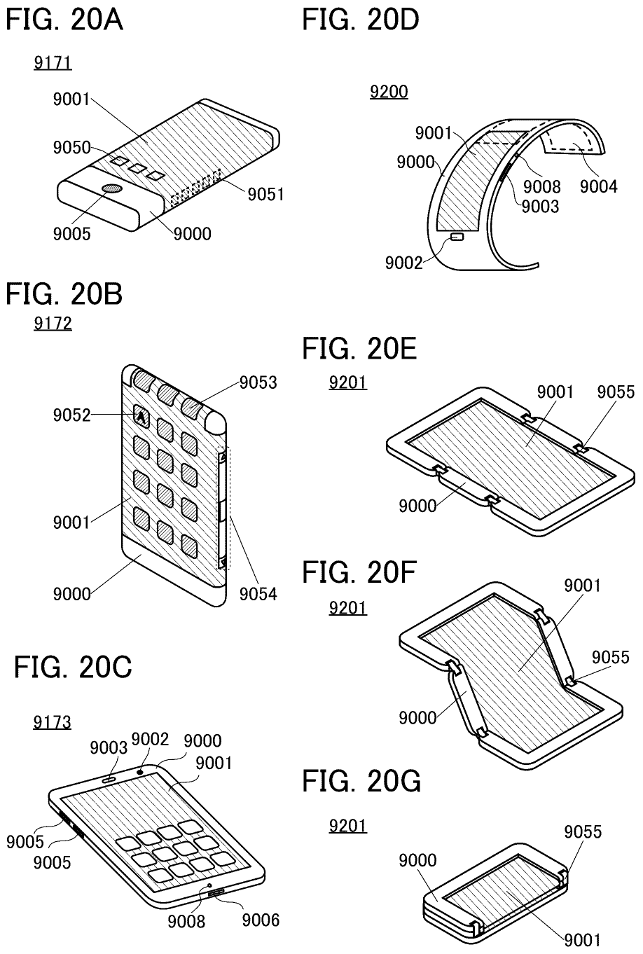

Electronic appliances illustrated in FIGS. 20A to 20G include a housing 9000, a display portion 9001, a speaker 9003, an operation key 9005 (including a power switch or an operation switch), a connection terminal 9006, a sensor 9007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 9008, and the like.

The electronic appliances illustrated in FIGS. 20A to 20G have a variety of functions. For example, the electronic appliances can have a function of displaying a variety of information (e.g., a still image, a moving image, and a text image) on the display portion, a touch panel function, a function of displaying a calendar, date, time, and the like, a function of controlling processing with use of a variety of software (programs), a wireless communication function, and a function of reading out and processing a program or data stored in a recording medium.

The electronic appliances illustrated in FIGS. 20A to 20G are described in detail below.

FIG. 20A is a perspective view of a portable information terminal 9171. The portable information terminal 9171 can be used as a smartphone, for example. The portable information terminal 9171 may include the speaker 9003, the connection terminal 9006, the sensor 9007, or the like. The portable information terminal 9171 can display text and image information on its plurality of surfaces. FIG. 20A illustrates an example where three icons 9050 are displayed. Furthermore, information 9051 indicated by dashed rectangles can be displayed on another surface of the display portion 9001. Examples of the information 9051 include notification of reception of an e-mail, an SNS message, an incoming call, or the like, a title and a sender of an e-mail, an SNS message, or the like, a date, time, a remaining battery power, and a radio field intensity. Alternatively, the icon 9050 or the like may be displayed at the position where the information 9051 is displayed.

FIG. 20B is a perspective view of a portable information terminal 9172. The portable information terminal 9172 has a function of displaying information on three or more surfaces of the display portion 9001. In the example illustrated here, information 9052, information 9053, and information 9054 are displayed on different surfaces. For example, the user of the portable information terminal 9172 can check the information 9053 displayed such that it can be seen from above the portable information terminal 9172, with the portable information terminal 9172 put in a breast pocket of his/her clothes.

FIG. 20C is a perspective view of a tablet terminal 9173. The tablet terminal 9173 is capable of executing a variety of applications such as mobile phone calls, e-mailing, viewing and editing texts, music reproduction, Internet communication, and a computer game, for example. The tablet terminal 9173 includes the display portion 9001, a camera 9002, the microphone 9008, and the speaker 9003 on the front surface of the housing 9000; the operation keys 9005 as buttons for operation on the left side surface of the housing 9000; and the connection terminal 9006 on the bottom surface of the housing 9000.

FIG. 20D is a perspective view of a watch-type portable information terminal 9200. The portable information terminal 9200 can be used as a Smartwatch (registered trademark), for example. The portable information terminal 9200 may include the operation key 9005 as a button for operation on the left side surface of the housing 9000 and the sensor 9007 on the bottom surface of the housing 9000. Although the housing 9000 having a curved bangle shape is illustrated as an example, a belt or the like may be used in combination with the housing 9000 to make the portable information terminal 9200 wearable. The display surface of the display portion 9001 is curved, and an image can be displayed on the curved display surface. A power storage device 9004 may have a curved shape along the housing 9000. The power storage device 9004 has flexibility and can be bent in accordance with a change in shape when the user puts on or takes off the portable information terminal 9200. Note that a charge control IC connected to the power storage device 9004 may be provided. Furthermore, for example, mutual communication between the portable information terminal 9200 and a headset capable of wireless communication can be performed, and thus hands-free calling is possible. The portable information terminal 9200 can perform mutual data transmission wirelessly with another information terminal and can be charged with wireless power feeding. Note that the connection terminal 9006 may be provided in the housing 9000 so that data transmission and charging operation may be performed by wire.

FIGS. 20E to 20G are perspective views of a foldable portable information terminal 9201. FIG. 20E is a perspective view showing the portable information terminal 9201 that is opened. FIG. 20G is a perspective view showing the portable information terminal 9201 that is folded. FIG. 20F is a perspective view showing the portable information terminal 9201 that is shifted from one of the states in FIGS. 20E and 20G to the other. The portable information terminal 9201 is highly portable when folded. When the portable information terminal 9201 is opened, a seamless large display region is highly browsable. The display portion 9001 of the portable information terminal 9201 is supported by three housings 9000 joined together by hinges 9055. The display portion 9001 can be folded with a radius of curvature greater than or equal to 0.1 mm and less than or equal to 150 mm, for example.

This embodiment can be combined as appropriate with the other embodiments or the examples. In this specification, in the case where a plurality of structure examples are shown in one embodiment, the structure examples can be combined as appropriate.

### Example 1

### Synthesis Example 1

In this example, a method for synthesizing 2,2′-(1,3-phenylene)bis[9-(1-pyrrolidinyl)-1,10-phenanthroline](abbreviation: mPrdPhen2P) represented by Structural Formula (100) in Embodiment 1 is described. The structure of mPrdPhen2P is shown below.

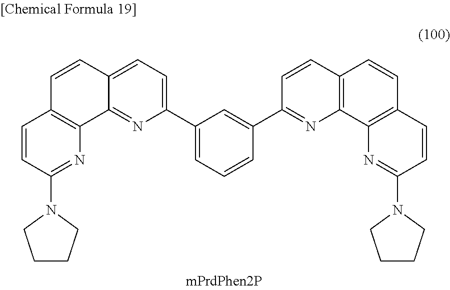 [SMILES: c1cc(-c2ccc3ccc4ccc(N5CCCC5)nc4c3n2)cc(-c2ccc3ccc4ccc(N5CCCC5)nc4c3n2)c1]

### Step 1: Synthesis of 2,2′-(1,3-phenylene)bis(9-chloro-1,10-phenanthroline)

To a three-neck flask were added 7.42 g (29.8 mmol) of 2,9-dichloro-1,10-phenanthroline, 1.65 g (9.95 mmol) of 1,3-phenylenediboronic acid, 8.23 g (59.6 mmol) of potassium carbonate, 49 mL of toluene, 17 mL of ethanol, and 30 mL of water, and the mixture was stirred under reduced pressure to be degassed. To this mixture was added 0.335 g (0.290 mmol) of tetrakis(triphenylphosphine)palladium(0), and the mixture was stirred at 100° C. for 6 hours under a nitrogen stream. After the stirring, the mixture was cooled down to room temperature, and a precipitated solid in the mixture was collected by suction filtration. This solid was purified by silica gel column chromatography (developing solvent: dichloromethane is used first and a mixed solvent of dichloromethane and chloroform (10:1) is used next). The obtained fraction was concentrated to give a solid. Ethyl acetate and hexane were added to the solid, irradiation with ultrasonic waves was performed, and the solid was collected by suction filtration to give 2.76 g of a target white solid in a yield of 55.2%). The synthesis scheme of 2,2′-(1,3-phenylene)bis(9-chloro-1,10-phenanthroline) is shown in Formula (a-1) below.

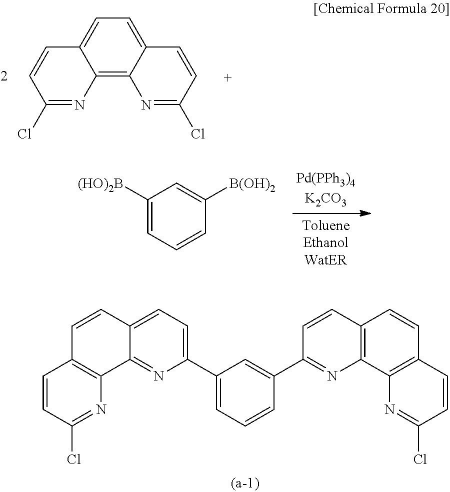 [SMILES: Clc1ccc2ccc3ccc(Cl)nc3c2n1 ; OB(O)c1cccc(B(O)O)c1 ; Clc1ccc2ccc3ccc(-c4cccc(-c5ccc6ccc7ccc(Cl)nc7c6n5)c4)nc3c2n1]

### Step 2: Synthesis of mPrdPhen2P

To a three-neck flask were added 2.76 g (5.48 mmol) of 2,2′-(1,3-phenylene)bis(9-chloro-1,10-phenanthroline), 0.819 g (11.5 mmol) of 1H-pyrrolidine, and 2.50 g (16.4 mmol) of 1,8-diazabicyclo[5.4.0]-7-undecene (abbreviation: DBU (registered trademark)), and the mixture was stirred under a nitrogen stream at 100° C. for 6 hours. After the stirring, the mixture was cooled down to room temperature. A precipitated solid in this mixture was collected by suction filtration. To this solid, water was added, irradiation with ultrasonic waves was performed, and the solid was collected by suction filtration. Ethanol was added to this solid, irradiation with ultrasonic waves was performed, and the solid was collected by suction filtration, whereby 2.96 g of a target yellow solid was obtained in a yield of 94%. A synthesis scheme of mPrdPhen2P is shown in Formula (a-2) below.

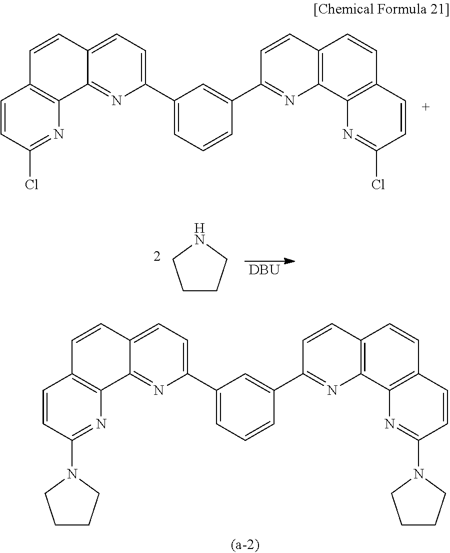

By a train sublimation method, the obtained yellow solid was purified. In the purification by sublimation, heating was performed at a heating temperature of 280° C. under a pressure of 4.2×10⁻² Pa for 48 hours. As a result, the target yellow solid was obtained.

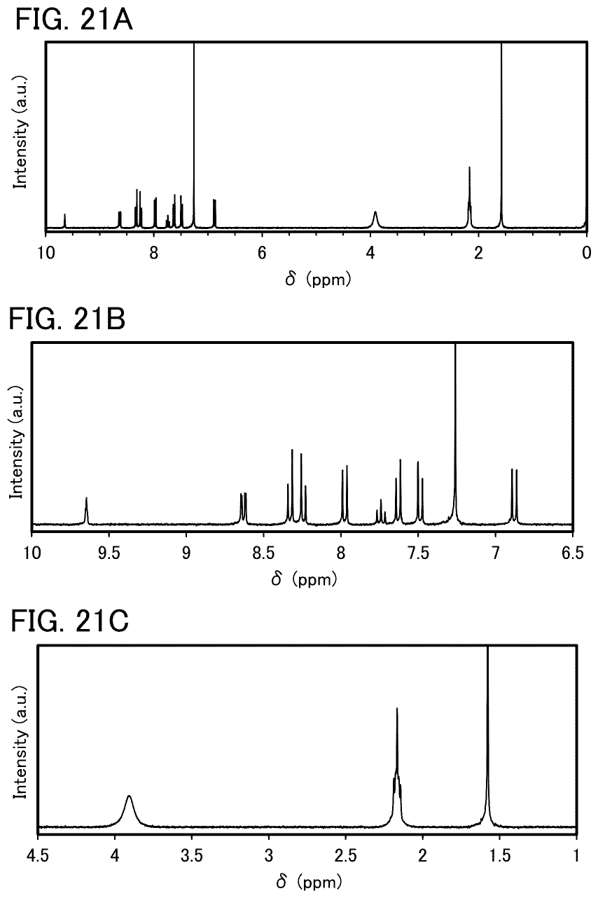

FIGS. 21A to 21C show ¹H NMR spectra of mPrdPhen2P after the purification by sublimation. FIG. 21B is an enlarged graph of FIG. 21A in the range from 6.5 ppm to 10 ppm, and FIG. 21C is an enlarged graph of FIG. 21A in the range from 1 ppm to 4.5 ppm. Results of ¹H NMR measurement are shown below. The results confirm that mPrdPhen2P was obtained.

¹H NMR (CDCl₃, 300 MHz): δ=9.65-9.64 (1H, m), 8.63 (2H, dd, J=7.7 Hz, 1.8 Hz), 8.33 (2H, d, J=8.4 Hz), 8.24 (2H, d, J=8.4 Hz), 7.97 (2H, d, J=8.8 Hz), 7.74 (1H, t, J=7.9 Hz), 7.63 (2H, d, J=8.4 Hz), 7.49 (2H, d, J=8.4 Hz), 6.88 (2H, d, J=8.8 Hz), 3.91 (8H, brs), 2.19-2.14 (8H, m).

The glass transition temperature (Tg) and the crystallization temperature (Tc) of mPrdPhen2P were measured. With a differential scanning calorimeter (DSC8500, PerkinElmer Japan), T_{g} and Tc were measured in a state where a powder was put on an aluminum cell and the temperature was increased at a rate of 40° C./min. T_{g} of mPrdPhen2P was 152° C. and Tc was not observed, which showed mPrdPhen2P has favorable heat resistance for use in a light-emitting device and low crystallinity.

### Example 2

In this example, light-emitting devices of one embodiment of the present invention and comparative light-emitting devices are described in detail. Structural Formulas of organic compounds mainly used in this example are shown below.

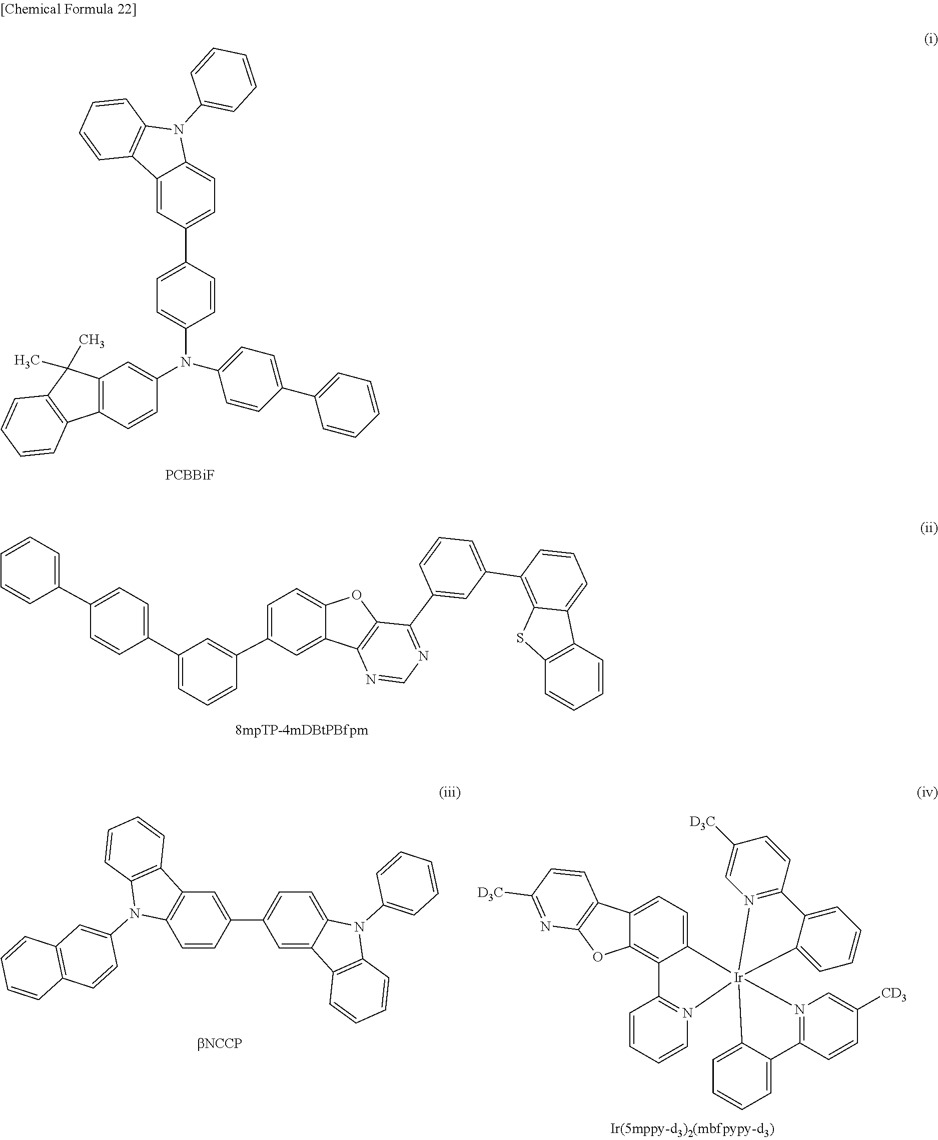

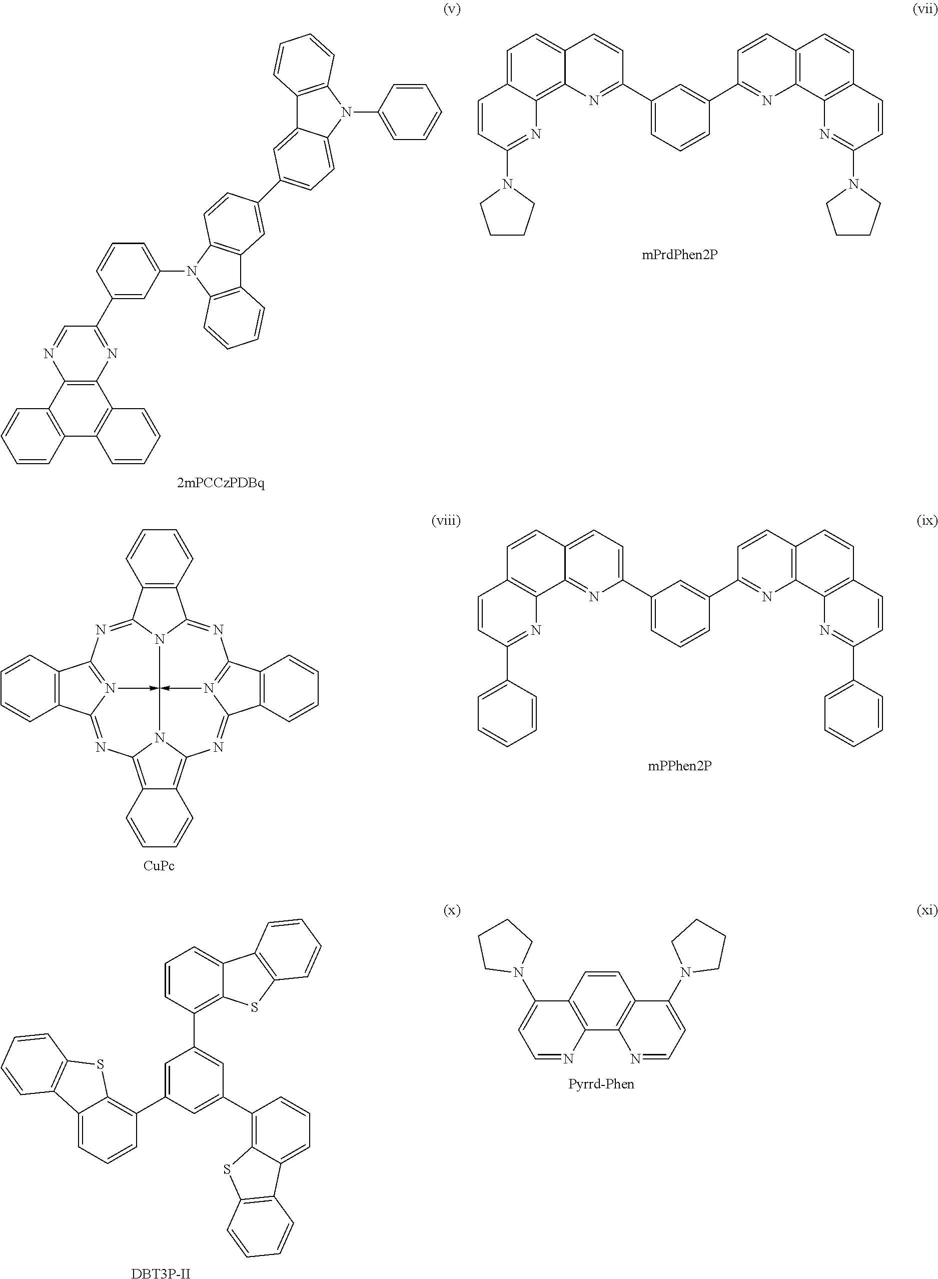

### (Fabrication Method of Light-Emitting Device 1-1)

First, 100-nm-thick silver serving as a reflective electrode and 85-nm-thick indium tin oxide including silicon oxide (JTSO) serving as a transparent electrode were stacked sequentially over a substrate by a sputtering method, whereby the first electrode 101 with a size of 2 mm×2 mm was formed. Note that the transparent electrode functions as an anode, and the transparent electrode and the reflective electrode are collectively regarded as the first electrode 101.

Next, in pretreatment for forming the light-emitting device over the substrate, the substrate surface was washed with water and baking was performed at 200° C. for 1 hour.

After that, the substrate was transferred into a vacuum evaporation apparatus where the pressure was reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Then, the substrate was fixed to a holder provided in the vacuum evaporation apparatus such that the surface on which the first electrode 101 was formed faced downward. Over the first electrode 101, N-(biphenyl-4-yl)-N-[4-(9-phenyl-9H-carbazol-3-yl)phenyl]-9,9-dimethyl-9H-fluoren-2-amine (abbreviation: PCBBiF) represented by Structural Formula (i) above and a fluorine-containing electron acceptor material with a molecular weight of 672 (OCHD-003) were deposited by co-evaporation to a thickness of 10 nm such that the weight ratio of PCBBiF to OCHD-003 was 1:0.03, whereby the hole-injection layer 111 was formed.

Over the hole-injection layer 111, PCBBiF was deposited by evaporation to a thickness of 85 nm, so that a first hole-transport layer was formed.

Then, over the first hole-transport layer, 8-(p-terphenyl-3-yl)-4-[3-(dibenzothiophen-4-yl)phenyl]-[1]benzofuro[3,2-d]pyrimidine (abbreviation: 8mpTP-4mDBtPBfpm) represented by Structural Formula (ii) above, 9-(2-naphthyl)-9′-phenyl-3,3′-bi-9H-carbazole (abbreviation: βNCCP) represented by Structural Formula (iii) above, and [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)) represented by Structural Formula (iv) above were deposited by co-evaporation to a thickness of 40 nm such that the weight ratio of 8mpTP-4mDBtPBfpm to βNCCP to Ir(5mppy-d₃)₂(mbfpypy-d₃) was 0.5:0.5:0.1, whereby a first light-emitting layer was formed.

After that, 2-{3-[3-(N-phenyl-9H-carbazol-3-yl)-9H-carbazol-9-yl]phenyl}dibenzo[f,h]quinoxaline (abbreviation: 2mPCCzPDBq) represented by Structural Formula (v) above was deposited by evaporation to a thickness of 10 nm to form a first electron-transport layer.

After the formation of the first electron-transport layer, 2,2′-(1,3-phenylene)bis[9-(1-pyrrolidinyl)-1,10-phenanthroline](abbreviation: mPrdPhen2P) represented by Structural Formula (vii) above and lithium oxide (Li₂O) were deposited by co-evaporation to a thickness of 5 nm such that the volume ratio of mPrdPhen2P to Li₂O was 1.0:0.02, whereby a first layer was formed. Subsequently, copper phthalocyanine (abbreviation: CuPc) represented by Structural Formula (viii) above was deposited by evaporation to a thickness of 2 nm to form a third layer, and then PCBBiF and OCHD-003 were deposited by co-evaporation to a thickness of 10 nm such that the weight ratio of PCBBiF to OCHD-003 was 1:0.15, whereby a second layer was formed. In this manner, an intermediate layer was formed.

Over the intermediate layer, PCBBiF was deposited to a thickness of 50 nm by evaporation, whereby a second hole-transport layer was formed.

Over the second hole-transport layer, 8mpTP-4mDBtPBfpm, βNCCP, and Ir(5mppy-d₃)₂(mbfpypy-d₃) were deposited by co-evaporation to a thickness of 40 nm such that the weight ratio of 8mpTP-4mDBtPBfpm to βNCCP to Ir(5mppy-d₃)₂(mbfpypy-d₃) was 0.5:0.5:0.1, whereby a second light-emitting layer was formed.

Then, 2mPCCzPDBq was deposited by evaporation to a thickness of 20 nm, and then 2,2′-(1,3-phenylene)bis(9-phenyl-1,10-phenanthroline) (abbreviation: mPPhen2P) represented by Structural Formula (ix) above was deposited by evaporation to a thickness of 20 nm, whereby a second electron-transport layer was formed.

After that, lithium fluoride (LiF) and ytterbium (Yb) were deposited by co-evaporation to a thickness of 1.5 nm such that the volume ratio of LiF to Yb was 1:0.5, and then silver (Ag) and magnesium (Mg) were deposited by co-evaporation to a thickness of 15 nm such that the volume ratio of Ag to Mg was 1:0.1, whereby the second electrode 102 was formed. Over the second electrode 102, 4,4′,4″-(benzene-1,3,5-triyl)tri(dibenzothiophene) (abbreviation: DBT3P-II) represented by Structural Formula (x) above was deposited by evaporation to a thickness of 70 nm as a cap layer to improve light extraction efficiency.

Then, the light-emitting device was sealed using a glass substrate in a glove box with a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for one hour. In this manner, the light-emitting device 1-1 was fabricated.

### (Fabrication Method of Light-Emitting Device 1-2)

A light-emitting device 1-2 was fabricated by heating the fabricated light-emitting device 1-1 at 130° C. for one hour.

### (Fabrication Method of Comparative Light-Emitting Device 1-1)

A comparative light-emitting device 1-1 was fabricated in a manner similar to that for the light-emitting device 1-1 except that mPrdPhen2P used for the first layer in the light-emitting device 1-1 was replaced with 4,7-di-1-pyrrolidinyl-1,10-phenanthroline (abbreviation: Pyrrd-Phen) represented by Structural Formula (xi) above and that the first hole-transport layer and the second hole-transport layer were deposited by evaporation to thicknesses of 90 nm and 55 nm, respectively.

### (Fabrication Method of Comparative Light-Emitting Device 1-2)

A comparative light-emitting device 1-2 was fabricated by heating the fabricated light-emitting device 1-1 at 130° C. for one hour.

Device structures of the light-emitting device 1 and the comparative light-emitting device 1 are shown in Tables 1 and 2.

TABLE 1

| | | |
|---|---|---|
| | Light-emitting | Comparative light- |
| | device 1-1 | emitting device 1-1 |
| Thickness | Light-emitting | Comparative light- |
| (nm) | device 1-2 | emitting device 1-2 |

| Cap layer | | 70 | DBT3P-II |
|---|---|---|---|
| Second electrode | | 15 | Ag:Mg (1:0.1) |
| | | 1.5 | LiF:Yb (1:0.5) |
| Second electron- | 2 | 20 | mPPhen2P |
| transport layer | 1 | 20 | 2mPCCzPDBq |
| Second light-emitting layer | | 40 | 8mpTP-4mDBtPBfpm:βNCCP:Ir(5mppy-d₃)₂(mbfpypy-d₃) |
| | | | (0.5:0.5:0.1) |
| Second hole-transport layer | | *3 | PCBBiF |
| Intermediate | Second layer | 10 | PCBBiF:OCHD-003 |
| layer | | | (1:0.15) |
| | Third layer | 2 | CuPc |
| | First layer | 5 | *1:Li₂O |
| | | | (1.0:0.02) |

| First electron-transport layer | 10 | 2mPCCzPDBq |
|---|---|---|
| First light-emitting layer | 40 | 8mpTP-4mDBtPBfpm:βNCCP:Ir(5mppy-d₃)₂(mbfpypy-d₃) |
| | | (0.5:0.5:0.1) |
| First hole-transport layer | *2 | PCBBiF |
| Hole-injection layer | 10 | PCBBiF:OCHD-003 |
| | | (1:0.03) |
| First electrode | 85 | ITSO |
| | 100 | Ag |

TABLE 2

| | *1 | *2 | *3 |
|---|---|---|---|
| Light-emitting device 1-1 | mPrdPhen2P | 85 nm | 50 nm |
| Light-emitting device 1-2 | | | |
| Comparative light-emitting device 1-1 | Pyrrd-Phen | 90 nm | 55 nm |
| Comparative light-emitting device 1-2 | | | |

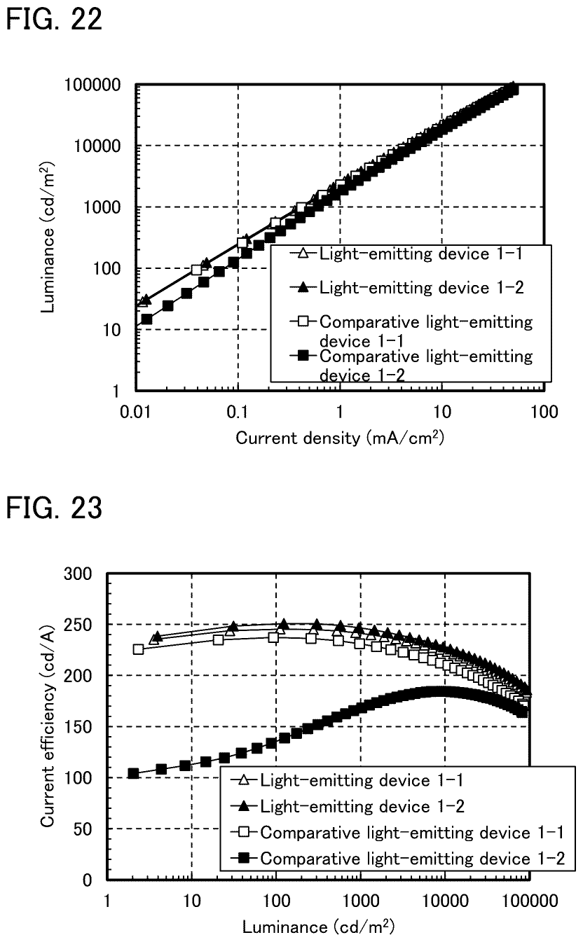

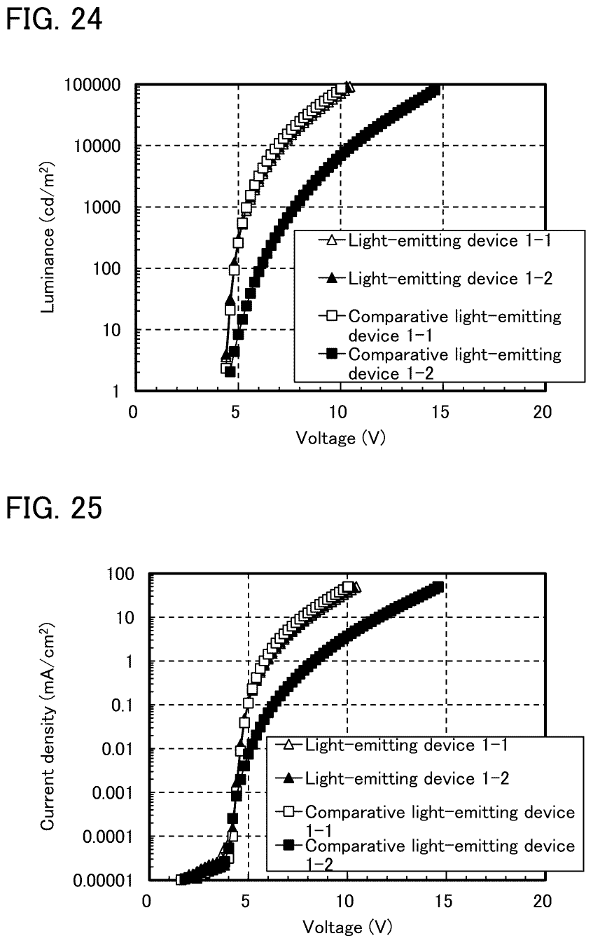

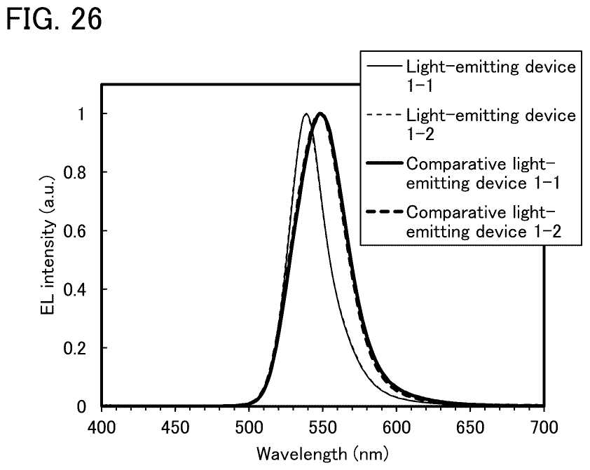

FIG. 22 shows luminance-current density characteristics of the light-emitting devices 1-1 and 1-2 and the comparative light-emitting devices 1-1 and 1-2. FIG. 23 shows current efficiency-luminance characteristics of the light-emitting devices 1-1 and 1-2 and the comparative light-emitting devices 1-1 and 1-2. FIG. 24 shows luminance-voltage characteristics of the light-emitting devices 1-1 and 1-2 and the comparative light-emitting devices 1-1 and 1-2. FIG. 25 shows current density-voltage characteristics of the light-emitting devices 1-1 and 1-2 and the comparative light-emitting devices 1-1 and 1-2. FIG. 26 shows electroluminescence spectra of the light-emitting devices 1-1 and 1-2 and the comparative light-emitting devices 1-1 and 1-2. Table 3 shows the main characteristics of the light-emitting devices 1-1 and 1-2 and the comparative light-emitting devices 1-1 and 1-2 at a luminance of about 1000 cd/m². The luminance, CIE chromaticity, and electroluminescence spectra were measured at normal temperature with a spectroradiometer (SR-UL1R, TOPCON TECHNOHOUSE).

TABLE 3

| | | | Current | | | Current |
|---|---|---|---|---|---|---|
| | Voltage | Current | density | Chromaticity | Chromaticity | efficiency |
| | (V) | (mA) | (mA/cm²) | x | y | (cd/A) |
| Light-emitting device 1-1 | 5.40 | 0.0144 | 0.360 | 0.285 | 0.694 | 242 |
| Light-emitting device 1-2 | 5.40 | 0.0155 | 0.388 | 0.286 | 0.693 | 247 |
| Comparative light-emitting device 1-1 | 5.40 | 0.0168 | 0.420 | 0.333 | 0.652 | 231 |
| Comparative light-emitting device 1-2 | 7.80 | 0.0245 | 0.613 | 0.327 | 0.658 | 168 |

FIG. 22 to FIG. 26 and Table 3 show that the light-emitting devices 1-1 and 1-2 and the comparative light-emitting device 1-1 each have high current efficiency and function as a tandem light-emitting device, whereas the comparative light-emitting device 1-2 has a significantly increased driving voltage and significantly decreased current efficiency.

The above results reveal that the light-emitting devices each using mPrdPhen2P as the organic compound represented by General Formula (G1) in Embodiment 1 for the first layer in the tandem light-emitting device do not exhibit deterioration in characteristics even when placed in a high-temperature environment and maintain favorable characteristics. Meanwhile, the light-emitting devices using Pyrrd-Phen for the first layer were found to suffer deterioration in characteristics by being exposed to a high-temperature environment.

Note that T_{g} of mPrdPhen2P was 152° C. and Tg of Pyrrd-Phen was 78° C. This result shows that the organic compound represented by General Formula (G1) in Embodiment 1 has favorable heat resistance and can be suitably used for the first layer in the tandem light-emitting device. Note that T_{g} was measured with a differential scanning calorimeter (DSC8500, PerkinElmer Japan).

As described above, since mPrdPhen2P, which is the organic compound represented by General Formula (G1) in Embodiment 1, has high heat resistance, a light-emitting device with favorable heat resistance can be provided.

### Example 3

In this example, the light-emitting device of one embodiment of the present invention will be described in detail. Structural Formulas of organic compounds mainly used in this example are shown below.

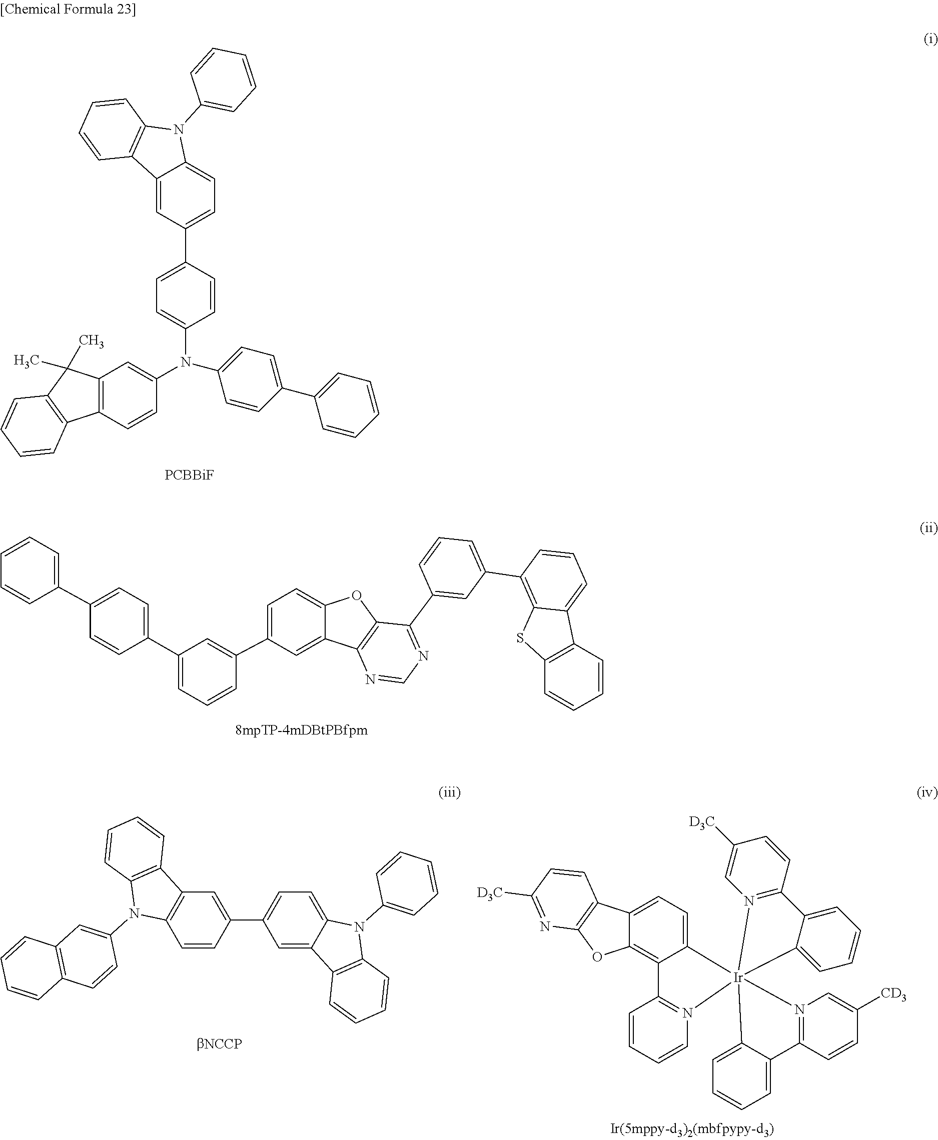

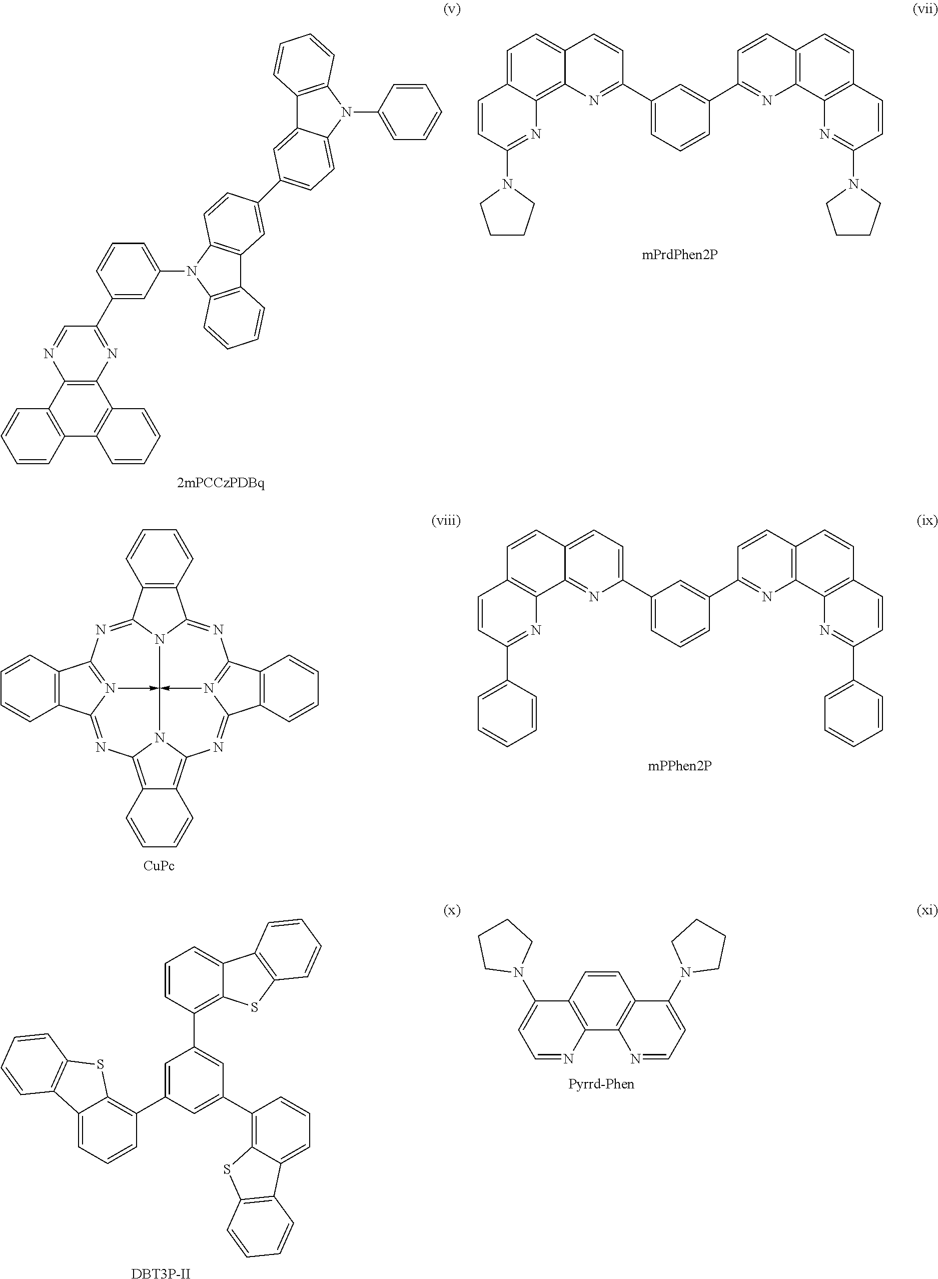

### (Fabrication Method of Light-Emitting Device 2)

First, 100-nm-thick silver serving as a reflective electrode and 85-nm-thick indium tin oxide including silicon oxide (JTSO) serving as a transparent electrode were stacked sequentially over a substrate by a sputtering method, whereby the first electrode 101 with a size of 2 mm×2 mm was formed. Note that the transparent electrode functions as an anode, and the transparent electrode and the reflective electrode are collectively regarded as the first electrode 101.

Next, in pretreatment for forming the light-emitting device over the substrate, the substrate surface was washed with water and baking was performed at 200° C. for 1 hour.

After that, the substrate was transferred into a vacuum evaporation apparatus where the pressure was reduced to approximately 1×10⁻⁴ Pa, and was subjected to vacuum baking at 170° C. for 30 minutes in a heating chamber of the vacuum evaporation apparatus, and then the substrate was cooled down for approximately 30 minutes.

Then, the substrate was fixed to a holder provided in the vacuum evaporation apparatus such that the surface on which the first electrode 101 was formed faced downward. Over the first electrode 101, N-(biphenyl-4-yl)-N-[4-(9-phenyl-9H-carbazol-3-yl)phenyl]-9,9-dimethyl-9H-fluoren-2-amine (abbreviation: PCBBiF) represented by Structural Formula (i) above and a fluorine-containing electron acceptor material with a molecular weight of 672 (OCHD-003) were deposited by co-evaporation to a thickness of 10 nm such that the weight ratio of PCBBiF to OCHD-003 was 1:0.03, whereby the hole-injection layer 111 was formed.

Over the hole-injection layer 111, PCBBiF was deposited by evaporation to a thickness of 85 nm, so that a first hole-transport layer was formed.

Then, over the first hole-transport layer, 8-(p-terphenyl-3-yl)-4-[3-(dibenzothiophen-4-yl)phenyl]-[1]benzofuro[3,2-d]pyrimidine (abbreviation: 8mpTP-4mDBtPBfpm) represented by Structural Formula (ii) above, 9-(2-naphthyl)-9′-phenyl-3,3′-bi-9H-carbazole (abbreviation: βNCCP) represented by Structural Formula (iii) above, and [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)) represented by Structural Formula (iv) above were deposited by co-evaporation to a thickness of 40 nm such that the weight ratio of 8mpTP-4mDBtPBfpm to βNCCP to Ir(5mppy-d₃)₂(mbfpypy-d₃) was 0.5:0.5:0.1, whereby a first light-emitting layer was formed.

After that, 2-{3-[3-(N-phenyl-9H-carbazol-3-yl)-9H-carbazol-9-yl]phenyl}dibenzo[f,h]quinoxaline (abbreviation: 2mPCCzPDBq) represented by Structural Formula (v) above was deposited by evaporation to a thickness of 10 nm to form the first electron-transport layer.

After the first electron-transport layer was formed, 2,2′-(1,3-phenylene)bis[9-(1-pyrrolidinyl)-1,10-phenanthroline](abbreviation: mPrdPhen2P) represented by Structural Formula (vii) above and indium (In) were deposited by co-evaporation to a thickness of 5 nm such that the volume ratio of mPrdPhen2P to In was 1.0:0.02, whereby the first layer was formed. Then, copper phthalocyanine (abbreviation: CuPc) represented by Structural Formula (vii) above was deposited by evaporation to a thickness of 2 nm, whereby the third layer was formed. Furthermore, PCBBiF and OCHD-003 were deposited by co-evaporation to a thickness of 10 nm such that the weight ratio of PCBBiF to OCHD-003 was 1:0.15, whereby the second layer was formed. In this manner, the intermediate layer was formed.

Over the intermediate layer, PCBBiF was deposited by evaporation to a thickness of 50 nm, whereby the second hole-transport layer was formed.

Over the second hole-transport layer, 8mpTP-4mDBtPBfpm, βNCCP, and Ir(5mppy-d3)₂(mbfpypy-d₃) were deposited by co-evaporation to a thickness of 40 nm such that the weight ratio of 8mpTP-4mDBtPBfpm to βNCCP to Ir(5mppy-d₃)₂(mbfpypy-d₃) was 0.5:0.5:0.1, whereby the second light-emitting layer was formed.

Then, 2mPCCzPDBq was deposited by evaporation to a thickness of 20 nm, and then 2,2′-(1,3-phenylene)bis(9-phenyl-1,10-phenanthroline) (abbreviation: mPPhen2P) represented by Structural Formula (ix) above was deposited by evaporation to a thickness of 20 nm, whereby the second electron-transport layer was formed.

A sample in which the components up to and including the second electron-transport layer had been formed was exposed to an air atmosphere for one hour. After that, the sample was subjected to heating (vacuum baking) at 100° C. for one hour in a heating chamber of the vacuum evaporation apparatus where the pressure was reduced to approximately 1×10⁻⁴ Pa.

After that, lithium fluoride (LiF) and ytterbium (Yb) were deposited by co-evaporation to a thickness of 1.5 nm such that the volume ratio of LiF to Yb was 1:0.5, and then silver (Ag) and magnesium (Mg) were deposited by co-evaporation to a thickness of 15 nm such that the volume ratio of Ag to Mg was 1:0.1, whereby the second electrode 102 was formed. Over the second electrode 102, 4,4′,4″-(benzene-1,3,5-triyl)tri(dibenzothiophene) (abbreviation: DBT3P-II) represented by Structural Formula (x) above was deposited by evaporation to a thickness of 70 nm as a cap layer to improve light extraction efficiency.

Then, the light-emitting device was sealed using a glass substrate in a glove box with a nitrogen atmosphere so as not to be exposed to the air. Specifically, a UV curable sealing material was applied to surround the device, only the sealing material was irradiated with UV while the light-emitting device was not irradiated with the UV, and heat treatment was performed at 80° C. under an atmospheric pressure for one hour. In this manner, the light-emitting device 2 was fabricated.

### (Fabrication Method of Comparative Light-Emitting Device 2)

The comparative light-emitting device 2 was fabricated in a manner similar to that for the light-emitting device 2 except that mPrdPhen2P used for the first layer in the light-emitting device 2 was replaced with mPPhen2P.

Device structures of the light-emitting device 2 and the comparative light-emitting device 2 are shown in Table 4.

TABLE 4

| | | |
|---|---|---|
| Thickness | Light-emitting | Comparative light- |
| (nm) | device 2 | emitting device 2 |

| Cap layer | 70 | DBT3P-II |
|---|---|---|
| Second electrode | 15 | Ag:Mg (1:0.1) |
| | 1.5 | LiF:Yb (1:0.5) |

Exposure to air and vacuum baking

| Second electron- | 2 | 20 | mPPhen2P | |
|---|---|---|---|---|
| transport layer | 1 | 20 | 2mPCCzPDBq | |
| Second light-emitting layer | | 40 | 8mpTP-4mDBtPBfpm:βNCCP:Ir(5mppy-d₃)₂(mbfpypy-d₃) | |
| | | | (0.5:0.5:0.1) | |
| Second hole-transport layer | | 50 | PCBBiF | |
| Intermediate | Second layer | 10 | PCBBiF:OCHD-003 | |
| layer | | | (1:0.15) | |
| | Third layer | 2 | CuPc | |
| | First layer | 5 | mPrdPhen2P:In | mPPhen2P:In |
| | | | (1.0:0.02) | (1.0:0.02) |

| | | |
|---|---|---|
| First electron-transport layer | 10 | 2mPCCzPDBq |
| First light-emitting layer | 40 | 8mpTP-4mDBtPBfpm:βNCCP:Ir(5mppy-d₃)₂(mbfpypy-d₃) |
| | | (0.5:0.5:0.1) |
| First hole-transport layer | 85 | PCBBiF |
| Hole-injection layer | 10 | PCBBiF:OCHD-003 |
| | | (1:0.03) |
| First electrode | 85 | ITSO |
| | 100 | Ag |

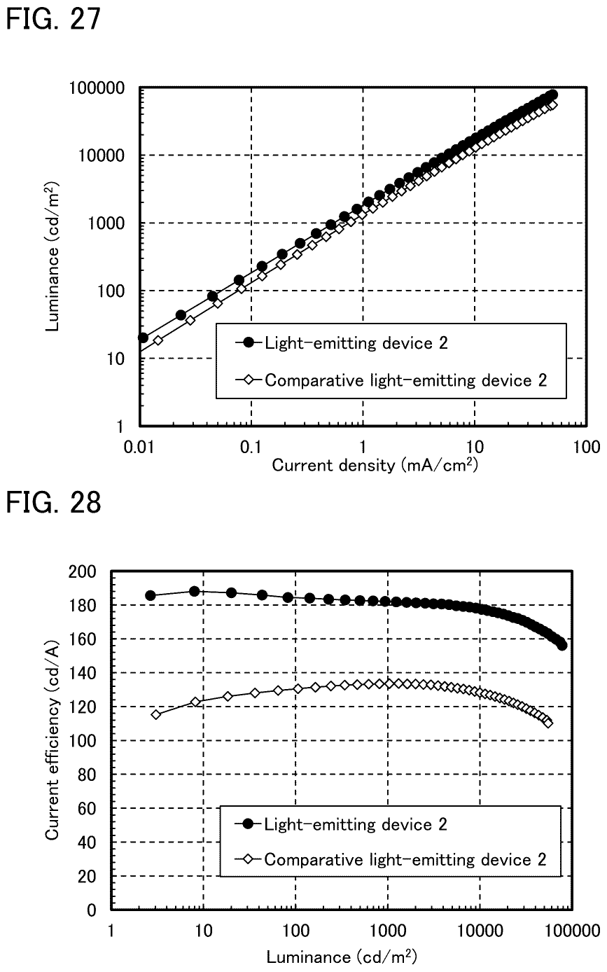

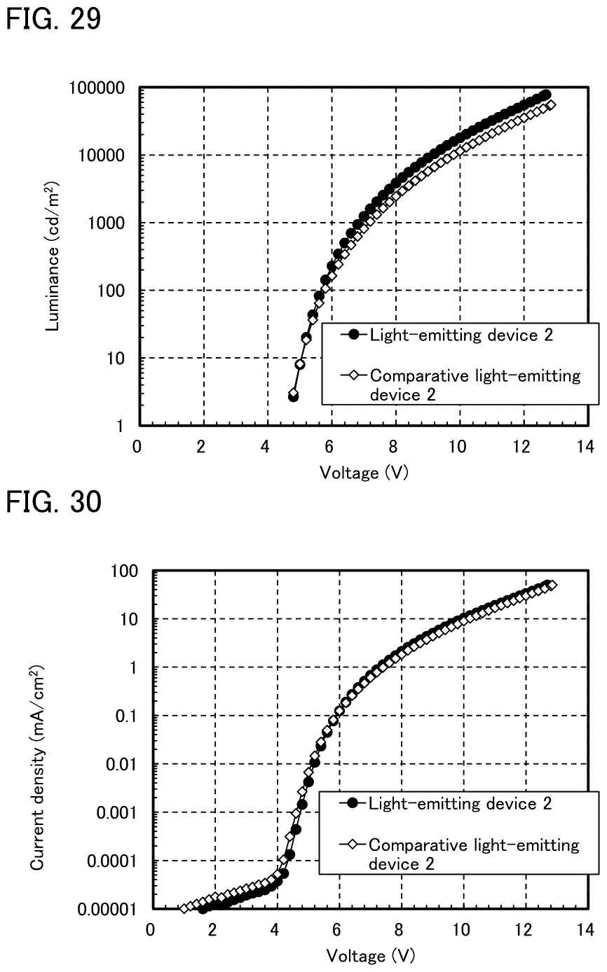

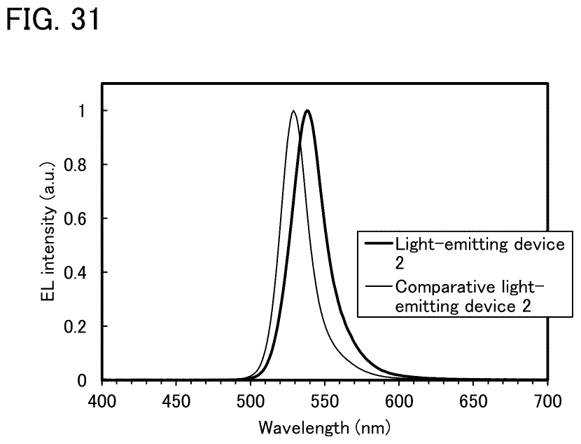

FIG. 27 shows luminance-current density characteristics of the light-emitting device 2 and the comparative light-emitting device 2. FIG. 28 shows current efficiency-luminance characteristics of the light-emitting device 2 and the comparative light-emitting device 2. FIG. 29 shows luminance-voltage characteristics of the light-emitting device 2 and the comparative light-emitting device 2. FIG. 30 shows current density-voltage characteristics of the light-emitting device 2 and the comparative light-emitting device 2. FIG. 31 shows electroluminescence spectra of the light-emitting device 2 and the comparative light-emitting device 2. Table 5 shows the main characteristics of the light-emitting device 2 and the comparative light-emitting device 2 at a luminance of about 1000 cd/m². The luminance, CIE chromaticity, and electroluminescence spectra were measured at normal temperature with a spectroradiometer (SR-UL1R, TOPCON TECHNOHOUSE).

TABLE 5

| | | | Current | | | Current |
|---|---|---|---|---|---|---|
| | Voltage | Current | density | Chromaticity | Chromaticity | efficiency |
| | (V) | (mA) | (mA/cm²) | x | y | (cd/A) |
| Light-emitting device 2 | 6.80 | 0.0206 | 0.515 | 0.268 | 0.709 | 182 |
| Comparative light-emitting device 2 | 7.20 | 0.0312 | 0.779 | 0.204 | 0.753 | 133 |

FIG. 27 to FIG. 31 and Table 5 show that the light-emitting device 2 using, for the intermediate layer, mPrdPhen2P, which is the organic compound represented by General Formula (G1) in Embodiment 1 exhibits favorable characteristics even after exposure to the air and heating. Meanwhile, the comparative light-emitting device 2 using, for the intermediate layer, mPPhen2P having a structure similar to that of the organic compound represented by General Formula (G1) in Embodiment 1 is found to have lower current efficiency and a higher driving voltage than the light-emitting device 2.

As described above, in the comparative light-emitting device 2, as a result of a decrease in the interaction between mPPhen2P and In due to the air exposure, the electron-injection property of the intermediate layer is decreased. On the other hand, in the light-emitting device 2, the electron density of a nitrogen atom can be increased owing to the aliphatic cyclic amino group in mPrdPhen2P and thus, the interaction with In is strong, so that the light-emitting device can be less likely to be affected by exposure to the air.

This application is based on Japanese Patent Application Serial No. 2024-188212 filed with Japan Patent Office on Oct. 25, 2024, the entire contents of which are hereby incorporated by reference.

What is claimed is:

1. An organic compound represented by General Formula (G1), 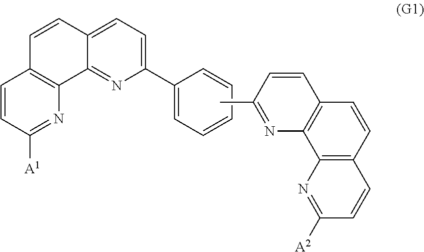
wherein each of A¹ and A² independently represents an aliphatic cyclic amino group represented by General Formula (g1), 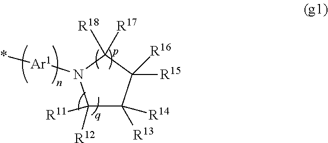
wherein R¹¹ to R¹⁸ each independently represent any one of hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein p and q each independently represent 0 to 3,
wherein any two of R¹¹ to R¹⁸ are bonded to each other to form a ring or not bonded to each other,
wherein the aliphatic cyclic amino group represented by the General Formula (g1) are condensed with an aromatic ring having 6 to 10 carbon atoms or not condensed with the aromatic ring,
wherein Ar¹ represents any one of a substituted or unsubstituted alkylene group having 1 to 3 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms,
wherein n represents an integer of 0 to 3, and
wherein when n is 2 or more, a plurality of Ar¹s are the same or different from each other.

2. An organic compound represented by General Formula (G2), 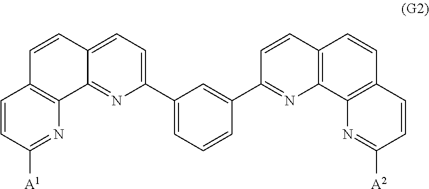
wherein A¹ and A² each independently represent a group represented by General Formula (g1), 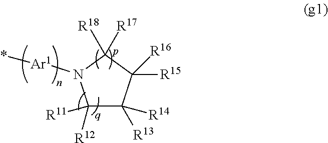
wherein R¹¹ to R¹⁸ each independently represent any one of hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein p and q each independently represent 0 to 3,
wherein any two of R¹¹ to R¹⁸ are bonded to each other to form a ring or not bonded to each other,
wherein the group represented by the General Formula (g1) are condensed with an aromatic ring having 6 to 10 carbon atoms or not condensed with the aromatic ring,
wherein Ar¹ represents any one of a substituted or unsubstituted alkylene group having 1 to 3 carbon atoms, a substituted or unsubstituted cycloalkylene group having 3 to 10 carbon atoms, a substituted or unsubstituted divalent aromatic hydrocarbon group having 6 to 25 carbon atoms, and a substituted or unsubstituted divalent heterocyclic group having 1 to 25 carbon atoms,
wherein n represents an integer of 0 to 3, and
wherein when n is 2 or more, a plurality of Ar¹s are the same or different from each other.

3. The organic compound according to claim 1,
wherein n in the General Formula (g1) is 0.

4. The organic compound according to claim 1,
wherein the R¹¹, the R¹², the R¹⁷, and the R¹⁸ each represent hydrogen.

5. The organic compound according to claim 3,
wherein the p and the q are 1.

6. An organic compound represented by Structural Formula (100)

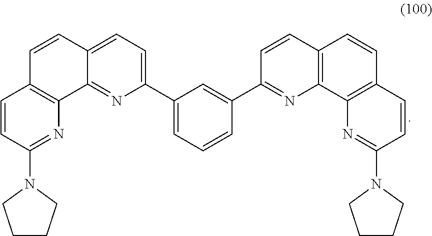 [SMILES: c1cc(-c2ccc3ccc4ccc(N5CCCC5)nc4c3n2)cc(-c2ccc3ccc4ccc(N5CCCC5)nc4c3n2)c1]

7. The organic compound according to claim 2,
wherein the R¹¹, the R¹², the R¹⁷, and the R¹⁸ each represent hydrogen.

8. The organic compound according to claim 7,
wherein the p and the q are 1.

9. A light-emitting device comprising the organic compound according to claim 1.

10. A light-emitting device comprising the organic compound according to claim 2.

11. A light-emitting device comprising the organic compound according to claim 6.
